(19) United States
(12) Patent Application Publication
(10) Pub. No.: US 2023/0357856 A1
(43) Pub. Date: Nov. 9, 2023

(54) METHODS AND COMPOSITIONS FOR PROGNOSING GLIOBLASTOMA OR BREAST CANCER

(71) Applicant: The Johns Hopkins University, Baltimore, MD (US)

(72) Inventors: Konstantinos Konstantopoulos, Ellicott, MD (US); Bin Sheng Wong, Baltimore, MD (US); Christopher Yankaskas, Baltimore, MD (US)

(21) Appl. No.: 18/026,058

(22) PCT Filed: Sep. 14, 2021
(86) PCT No.: PCT/US2021/050245

§ 371 Date: Mar. 13, 2023

Related U.S. Application Data
(60) Provisional application No. 63/077,828, filed on Sep. 14, 2020.

(51) Int. Cl. C12Q 1/6886; C12Q 1/24

(52) U.S. Cl. CPC C12Q 1/6886; C12Q 1/24; C12Q 2600/158; G01N 2800/7028

(57) ABSTRACT

Disclosed herein are methods for identifying a subject with an increased risk of short survival and/or recurrence of glioblastoma or breast cancer, the methods comprising: a) obtaining a brain or breast tissue sample or having obtained a brain or breast tissue sample from a subject; b) determining gene expression levels of one or more of PLK3, FOSL1, ADM, PLAU, VEGFA, NQOI, HMOX1, PGKI, and HPCAL1 in the sample from the subject. Also disclosed herein are diagnostic devices comprising one or more biomarkers, wherein the biomarkers are one or more of PLK3, FOSL1, ADM, PLAU, VEGFA, NQOI, HMOX1, PGKI, and HPCAL1; and a gene expression panel consisting of primers or probes for detecting one or more of DUSP5, PLK3, PPPIR15A, FOSL1, CDKNIA, KLF6, VDR, ARL4C, ADM, PLAU, VEGFA, NQOI, HMOX1, PGKI, LITAF, HPCALI and FTH1 in a sample, and methods for assessing risk of recurrence of glioblastoma or breast cancer in a subject.

### CROSS REFERENCE TO RELATED APPLICATIONS

This application claims the benefit of U.S. Provisional Application No. 63/077,828, filed on Sep. 14, 2020. The content of this earlier filed application is hereby incorporated by reference herein in its entirety.

### STATEMENT REGARDING FEDERALLY FUNDED RESEARCH

This invention was made with government support under grant number CA216855 awarded by the National Cancer Institute. The government has certain rights in the invention.

### FIELD OF THE INVENTION

The present disclosure relates to compositions and methods for determining the recurrence time and survival in subjects diagnosed with primary glioblastoma or breast cancer.

### BACKGROUND

Glioblastoma (GBM) is the most common and aggressive type of primary brain cancer in adults, accounting for about 15-20% of all brain malignancies (Ostrom, Q. T. et al., Neuro Oncol 20, iv1-iv86, (2018)). Owing to its highly proliferative and infiltrative nature, the median survival of GBM patients is approximately 14.6 months, with less than 5% of patients surviving past 5 years (Ostrom, Q. T. et al., Neuro Oncol 20, iv1-iv86, (2018)), and Stupp, R. et al., N Engl J Med 352, 987-996, (2005)). GBMs invade locally into the surrounding brain parenchyma and frequently spread to the contralateral hemisphere through the corpus callosum, thereby confounding local therapy and rendering gross total resection nearly impossible²⁻⁴. As a result, despite aggressive radical surgical resection coupled with concurrent chemo- and radio-therapy, GBMs remain incurable and recur frequently (Chaichana, K. L. et al., J Neurosurg 118, 812-820 (2013)). Presently, there is no effective in vitro platforms that can rapidly measure complex cellular phenotypic traits and predict patient-specific clinical outcomes.

### SUMMARY

Disclosed herein are methods for identifying a subject with an increased risk of short survival and/or recurrence of glioblastoma, the methods comprising: a) obtaining a brain tissue sample or having obtained a brain tissue sample from a subject; b) determining gene expression levels of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample from the subject; and c) optionally comparing the level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 to a predetermined reference level, identifying a subject with an increased risk of short survival and/or recurrence of glioblastoma when the level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample is determined to be higher than a predetermined reference level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample.

Disclosed herein are methods of treating a subject with an increased risk of recurrence of glioblastoma, the methods comprising: a) obtaining a brain tissue sample or having obtained a brain tissue sample from a subject; b) determining gene expression levels of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample from the subject; c) identifying the subject to have an increased risk of recurrence of glioblastoma when the level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample is determined to be higher than a predetermined reference level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1; and d) administering a suitable cancer therapeutic to the subject having an increased risk of recurrence of glioblastoma.

Disclosed herein are diagnostic devices, comprising biomarkers, wherein the biomarkers are DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1.

Disclosed herein are gene expression panels for assessing risk of recurrence of glioblastoma, consisting of primers or probes for amplifying or detecting DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1.

Disclosed herein are methods for identifying a subject with an increased risk of short survival and/or recurrence of breast cancer, the methods comprising: a) obtaining a breast tissue sample or having obtained a breast tissue sample from a subject; b) determining gene expression levels of PGK1, NQO1, HMOX1, VEGFA, ADM, HPCAL1, PLK3, FOSL1, and PLAU in the sample from the subject; and c) optionally comparing the level of gene expression PGK1, NQO1, HMOX1, VEGFA, ADM, HPCAL1, PLK3, FOSL1, and PLAUto a predetermined reference level, identifying a subject with an increased risk of short survival and/or recurrence of breast cancer when the level of gene expression of PGK1, NQO1, HMOX1, VEGFA, ADM, HPCAL1, PLK3, FOSL1, and PLAUin the sample is determined to be higher than a predetermined reference level of gene expression PGK1, NQO1, HMOX1, VEGFA, ADM, HPCAL1, PLK3, FOSL1, and PLAU in the sample.

Disclosed herein are methods of treating a subject with an increased risk of recurrence of breast cancer, the methods comprising: a) obtaining a breast tissue sample or having obtained a breast tissue sample from a subject; b) determining gene expression levels of PGK1, NQO1, HMOX1, VEGFA, ADM, HPCAL1, PLK3, FOSL1, and PLAU in the sample from the subject; c) identifying the subject to have an increased risk of recurrence of breast cancer when the level of gene expression of PGK1, NQO1, HMOX1, VEGFA, ADM, HPCAL1, PLK3, FOSL1, and PLAU in the sample is determined to be higher than a predetermined reference level of gene expression of PGK1, NQO1, HMOX1, VEGFA, ADM, HPCAL1, PLK3, FOSL1, and PLAU; and d) administering a suitable cancer therapeutic to the subject having an increased risk of recurrence of breast cancer.

Disclosed herein are diagnostic devices, comprising biomarkers, wherein the biomarkers are PGK1, NQO1, HMOX1, VEGFA, ADM, HPCAL1, PLK3, FOSL1, and PLAU.

Disclosed herein are gene expression panels for assessing risk of recurrence of breast cancer, consisting of primers or probes for amplifying or detecting PGK1, NQO1, HMOX1, VEGFA, ADM, HPCAL1, PLK3, FOSL1, and PLAU.

Disclosed herein are methods of screening for a therapeutic agent, the methods comprising: a) placing a cell or a population of cells from a brain or breast tissue sample in an integrative microfluidic apparatus, wherein the integrative microfluidic apparatus comprises a migratory channel and a bifurcation point in the channel; b) determining whether the cell or the population of cells migrates through the migratory channel of the apparatus and to the bifurcation point of the channel in the presence and absence of the therapeutic agent; and c) determining that the therapeutic agent is an inhibitor of cancer cell migration when the cell or population of cells does not migrate through the migratory channel of the apparatus and to the bifurcation point of the channel in the presence of the therapeutic agent.

### BRIEF DESCRIPTION OF THE DRAWINGS

FIGS. 1A-C show that the Microfluidic Assay for Quantification of Cell Invasion (MAqCI) distinguishes patient-derived primary GBM cells based on their migratory and proliferative potentials. FIG. 1A is a schematic of MAqCI consisting of a series of 10 µm-tall and 400 µm-long Y-shape microchannels, with a 20 µm-wide feeder channel bifurcating to either a 10 µm- or 3 µm-wide branch. Inset: Representative time-lapse micrographs of GBM714 migrating in MAqCI. Lowly motile cells (top row) are defined as cells that remain in the feeder channels and fail to enter the bifurcations (blue and cyan triangles). Highly motile cells (bottom row) are defined as cells that traverse through the entire length of the feeder channel and enter either the 10 µm wide (red triangle) or 3 µm narrow branches (orange triangle). Duration between each frame is 4 h. FIG. 1B show representative epifluoresence images of Ki67-negative non-proliferative (top row) and Ki67-positive proliferative (bottom row) GBM965 that have migrated in MAqCI. The cells (white triangles) were immunostained for Ki67 (green) and counterstained for nucleus with Hoechst 33342 (blue). FIG. 1C is a bar graph showing the percentage of highly motile cells (1st row), percentage of narrow entry (2nd row), percentage of highly motile Ki67-positive cells (3rd row) and percentage of unsorted Ki67-positive cells (4th row) for a retrospective panel of 28 patient-derived primary GBM cells tested with MAqCI. Red bars represent cells that are derived from patients with short-term survival (<14.6 months, n=19). Blue bars represent cells that are derived from patients with long-term survival (>14.6 months, n=9). Data represent the mean±S.E.M. from n≥3 independent experiments.

FIGS. 2A-D show that migratory and proliferative potentials of GBMs correlate with patient survival. FIG. 2A shows that the retrospective GBM patient cohort is separated into short- (red bars, n=19) and long-term survivors (blue bars, n=9), and compared for their average percentage of highly motile cells (1st panel), narrow entry (2nd panel), highly motile Ki67-positive cells (3rd panel) and unsorted Ki67-positive cells (4th panel). * represents p<0.05 as assessed by unpaired student’s t-test. FIG. 2B shows linear regression analysis of GBM patient survival in months against percentages of highly motile cells (1st panel), narrow entry (2nd panel), highly motile Ki67-positive cells (3rd panel) and unsorted Ki67- positive cells (4th panel). Black solid line represents the best-fit line while black dotted line represents the 95% confidence interval. * represents p<0.05. Pearson’s correlation was used to assess the significance of the correlation. FIG. 2C shows Kaplan-Meier curves based on MAqCI measurements, comparing survival of the retrospective cohort as separated by percentages of highly motile cells (1st panel), narrow entry (2nd panel), highly motile Ki67-positive cells (3rd panel) and unsorted Ki67-positive cells (4th panel). ** represents p<0.01 and ns represents p>0.05 as assessed by two-tailed log-rank (Mantel-Cox) test. FIG. 2D shows receiver operating characteristic curves of classifying GBM patients into short- or long-term survivors based on percentages of highly motile cells (1st panel), narrow entry (2nd panel), highly motile Ki67-positive cells (3rd panel) and unsorted Ki67-positive cells (4th panel). Area under curve (AUC) was calculated to indicate the prognostic utility of the different classifiers.

FIGS. 3A-F show that combining migratory and proliferative indices into a single composite score maximizes the prognosis performance of MAqCI. FIG. 3A shows the values of composite MAqCI score computed with logistic regression by combining percentages of highly motile cells, narrow entry and highly motile Ki67-positive cells as independent predictors. FIG. 3B shows the mean composite MAqCI score of short- (red bar, n=19) versus long-term (blue bar, n=9) survivors. *** represents p<0.001 as assessed by unpaired student’s t-test. FIG. 3C shows the Linear regression analysis of GBM patient survival against composite MAqCI score. Black solid line represents the best-fit line while black dotted line represents the 95% confidence interval. *** represents p<0.001. Pearson’s correlation was used to assess the significance of the correlation. FIG. 3D shows the Kaplan-Meier curve based on composite MAqCI score, comparing survival of the retrospective cohort as separated by high (>0.7, n=17) or low (<0.5, n=11) composite MAqCI score. ** represents p<0.01 as assessed by two-tailed log-rank (Mantel-Cox) test. FIG. 3E shows the receiver operating characteristic curve of classifying GBM patients into short- or long-term survivors based on composite MAqCI score. AUC was calculated to indicate the prognostic utility of composite MAqCI score in classifying GBM patient into short- or long-term survivals. FIG. 3F shows heat maps summarizing the ability of individual MAqCI measurement metrics and composite MAqCI score in categorizing GBM patients into short- or long-term survivors. The 28 GBM patients are arranged in increasing order with survival (1st panel), percentage of highly motile cells (2nd panel), percentage of narrow entry (3rd panel) and percentage of highly motile Ki67- positive (4th panel) and composite MAqCI score (5th panel) of the 28 retrospective GBM patients as presented in a red-blue double gradient with white color set as the threshold. False positive (FP: patients who are incorrectly categorized as short-term survivors) and false negative (FN: patients who are incorrectly categorized as long-term survivors) are indicated.

FIGS. 4A-D show that MAqCI predicts GBM patient survival retrospectively and prospectively with high effectiveness. FIG. 4A shows the linear regression analysis of GBM patient time to recurrence in months against percentages of highly motile cells (1st panel), narrow entry (2nd panel) and highly motile Ki67-positive cells (3rd panel), and composite MAqCI score (4th panel). Black solid line represents the best-fit line while black dotted line represents the 95% confidence interval. * represents p<0.05 and ** represents p<0.01. Pearson’s correlation was used to assess the significance of the correlation. FIG. 4B shows the mean time to recurrence of low versus high percentages of highly motile cells (1st panel), narrow entry (2nd panel) and highly motile Ki67-positive cells (3rd panel), and composite MAqCI score (4th panel). ** represents p<0.01 and *** represents p<0.001 as assessed by unpaired student’s t-test. FIG. 4C shows the percentages of highly motile cells (1st panel), narrow entry (2nd panel) and highly motile Ki67-positive (3rd panel), and composite MAqCI score (4th panel) of 5 prospective patient-derived primary GBM cells. Data represent the mean±S.E.M. from n≥3 independent experiments. FIG. 4D shows the heat maps summarizing the individual MAqCI measurement metrics and composite MAqCI score as described in (FIG. 3A) for the 5 prospective patients (1st to 4th panels). MAqCI correctly matches the predicted survival (5th panel; red=predicted to be short-term survivors; blue=predicted to be long-term survivors) to the actual survival (6th panel; red=patient deceased before 14.6 months; blue=patient still alive after 14.6 months).

FIGS. 5A-F show the transcriptome differences between highly motile and unsorted bulk GBM cell subpopulations. FIG. 5A shows a PCA subplot of highly motile (M) and unsorted bulk (B) cell samples from GBM 965 and 897. FIG. 5B shows a volcano plot showing DEGs in the highly motile subpopulations, which were identified using FDR<0.1 as a cutoff. FIG. 5C shows a scatter plot for top 25 GOBP enrichment terms of DEGs at FDR (Benjamini)<0.05. FIG. 5D shows data from the 464 DEGs between the highly motile and unsorted bulk cell subpopulations, expression data for 261 DEGs were available and used to assess the survival time in a cohort of 523 GBM patients. Kaplan-Meier analysis revealed that upregulated DEGs with a padj<0.05 correlated with reduced GBM OS. FIGS. 5E and F show the results of using a collection of 17 upregulated DEGs whose individual expression patterns correlated with OS, a composite score was calculated for each patient. Patients were stratified based on median (E) or tercile (F) scores. Kaplan-Meier analysis showed significantly worse OS for this collection of 17 upregulated DEGs. Log-rank test was used to calculate P-value and hazard ratio (HR) in (D), (E) and (F).

FIG. 6 is a schematic of the clinical usage of MAqCI. Primary GBM specimens harvested from patient following surgical resection are allowed to migrate in MAqCI, which recapitulates aspects of the complex topography and the confining microenvironment that GBM invasion occurs natively in brain parenchyma. The migratory and proliferative potentials of the patient-derived GBM cells are measured and used to compute a composite MAqCI score, which is then subsequently used to predict patient prognosis and identify patient-specific effective therapies. Higher composite MAqCI score correlates with shorter-term progression-free survival and lower time to recurrence.

FIGS. 7A-B show the results of optimization of extracellular matrix coating and channel dimensions for MAqCI. FIG. 7A shows the percentage of highly motile cells for GBM965 in MAqCI coated with either 12 µg/ml laminin, 20 µg/ml collagen type I or 20 µg/ml fibronectin. ** represents p<0.01 and *** represents p<0.001 as accessed by One-Way ANOVA with Tukey multiple comparison post-hoc test. Data represent the mean±S.E.M. from n≥3 independent experiments. FIG. 7B shows the percentage of highly motile cells for GBM965, GBM1049 and GBM612 in MAqCI with either asymmetric 3 µm and 10 µm bifurcations (3/10, red bar) or symmetric 10 µm and 10 µm bifurcations (10/10, blue bar). Significance between the percentage of highly motile cells for each MAqCI channel design was assessed using unpaired student’s t-test for each cell line. Data represent the mean±S.E.M. from n≥3 independent experiments.

FIGS. 8A-G show that the demographic, tumor and surgical attributes do not correlate with GBM patient survival. FIG. 8A shows the Kaplan-Meier curve based on the 14.6 months median survival threshold for the retrospective GBM patient cohort as established by Stupp et al, comparing survivals between short- (<14.6 months, n=19, red line) and long-term survivors (>14.6 months, n=9, blue line). Grey line indicates the survival curve of 28 retrospective GBM patients. FIGS. 8B-F show Kaplan-Meier curves based on demographic, tumor and surgical data of the retrospective GBM patient cohort, comparing survivals of groups as divided by median age (B), gender (C), median pre-operation tumor volume (D), tumor spread (E) and number of surgical resections (F). These attributes fail to separate the population significantly (p>0.05) as assessed by two-tailed log-rank (Mantel-Cox) test. FIG. 8G shows the preoperative axial T1-weighted MRI with contrast (top panel) and Fluid Attenuated Invasion Recovery (FLAIR, bottom panel) images showing GBM lesions of short-term survivors (GBM960 and GBM965) and a long-term survivor (GBM940). White triangles represent the bilateral extension of GBM through the corpus callosum into the contralateral hemisphere known as the butterfly spread.

FIGS. 9A-E show threshold value for each MAqCI measurement metric and composite MAqCI score as predictors for GBM patient survival. FIGS. 9A-D show heat maps as presented in a red-blue double gradient (blue=minimum 0%, red=maximum 100%, white=base line 50%) indicating the values of sensitivity, specificity, positive predictive value (PPV), negative predictive value (NPV), accuracy and average of the above-mentioned five parameters of correctly categorizing the retrospective GBM samples into either short- or long-term survivors as one varies the threshold of percentages of highly motile cells (A),narrow entry (B), highly motile Ki67-positive cells (C) and unsorted Ki67-positive cells (D), and composite MAqCI score (E). An optimal threshold value was determined that maximizes the measures of performance. Crossed square represents an inability to compute a numerical value at that particular threshold value.

FIGS. 10A-B show the multiple linear regression of between GBM patient survival and MAqCI measurement metrics. FIG. 10A shows the correlation between actual survival outcomes in months for the retrospective GBM cohort and the predicted survival in months as calculated based on the coefficients of multiple linear regression analysis as tabulated in FIG. 10B using the percentages of highly motile cells, narrow entry and highly motile Ki67-positive cells as independent predictors. Black solid line represents the best-fit line while black dotted line represents the 95% confidence interval.*** represents p<0.001. Pearson’s correlation was used to assess the significance of the correlation.

FIGS. 11A-F show that MAqCI does not discriminate against demographic, surgical, tumor and clinical attributes of GBM patients. FIGS. 11A-G show the retrospective GBM patient cohort is separated into low and high groups based on the optimal threshold of percentages of highly motile cells (1st panel), narrow entry (2nd panel) and highly motile Ki67-positive (3rd panel) or composite MAqCI score (4th panel) and compared for their mean age (A), gender (B), Karnofsky Performance Status (KPS) score (C), pre-operative tumor volume (D), extent of resection (E) and tumor extension (multifocal versus unifocal) (F). Significance between low versus high groups for each classifier were assessed using an unpaired student’s t-test for the continuous variables and Fisher’s exact test for the categorical variables.

FIGS. 12A-E show that IDH1 mutation status does not significantly correlate with GBM patient survival. FIG. 12A shows Western blot panels of primary GBM cells derived from the retrospective patient cohort for the mutant form of IDH1 (IDH1R132H, top panel), total IDH1 (middle panel) and GAPDH (bottom panel) as housekeeping and loading control. The patients are classified as IDH1 mutant or IDH1 wild type (WT) based on the presence or absence of IDH1R132H bands. FIG. 12B shows the percentage of patients exhibiting either IDH1 WT or mutant of short- versus long-term survivors. Statistical significance was assessed by unpaired student’s t-test. FIG. 12C shows the mean GBM patient survival in months of patients exhibiting IDH1 WT or mutant. Statistical significance was assessed by unpaired student’s t-test. FIG. 12D shows the Kaplan-Meier curve based on IDH1 mutation status, comparing survival of the retrospective cohort as grouped into IDH1 WT (n=9) or IDH1 mutant (n=19). ns represents p>0.05 as assessed by two-tailed log-rank (Mantel-Cox) test. FIG. 12E shows the receiver operating characteristic curve of classifying GBM patients into short- or long-term survivors based on IDH1 mutation status. AUC was calculated to indicate the prognostic utility of IDH1 mutation status in classifying GBM patient into short- or long-term survivals.

FIGS. 13A-G show that the transwell-migration assay does not correlate with GBM patient survival. FIG. 13A shows the optimization of the cell index threshold and experiment duration for the transwell-migration assay to maximize the sensitivity (right panel), specificity (middle panel) and accuracy (left panel) of categorizing patients correctly into either short- versus long-term survivals based on the 14.6 months of median survival threshold for GBM patients as established by Stupp et al. FIG. 13B is a table summarizing the cell-index threshold and their corresponding experimental duration that achieve the most optimal survival classification. FIG. 13C shows the cell index of the 27 retrospective patient-derived primary GBM cells as measured by the transwell-migration assay at 24 h. Red bars represent cells that are derived from patients with short-term survival (<14.6 months, n=18). Blue bars represent cells that are derived from patients with long-term survival (>14.6 months, n=9). Data represent the mean±S.E.M. from n≥3 independent experiments. FIG. 13D shows the mean cell index of short- (red bar, n=18) versus long-term (blue bar, n=9) survivors. * represents p<0.05 as assessed by unpaired student’s t-test. FIG. 13E shows the linear regression analysis of GBM patient survival against cell index. Black solid line represents the best-fit line while black dotted line represents the 95% confidence interval. ns represents p>0.05. Pearson’s correlation was used to assess the significance of the correlation. FIG. 13F shows the Kaplan-Meier curve based on cell index, comparing survival of the retrospective cohort as separated by high (>0.15, n=2) or low (<0.15, n=25) cell index. ns represents p>0.05 as assessed by two-tailed log-rank (Mantel-Cox) test. FIG. 13G shows the receiver operating characteristic curve of classifying GBM patients into short- or long-term survivors based on cell index. AUC was calculated to indicate the prognostic utility of cell index in classifying GBM patient into short- or long-term survivals.

FIGS. 14A-C show the transcriptome differences between highly motile and unsorted bulk GBM cell subpopulations. FIG. 14A shows a PCA subplot of the highly motile (M) and unsorted bulk (FIG. 14B) cell samples from GBM 965 and 897. FIG. 13B shows a scree plot reporting the percentage of variance between sequencing datasets for highly motile and unsorted bulk cell specimens explained by each principle component. Cumulative explained variance indicated by red curve, with 9 PCs explaining 100% of the variation. FIG. 13A shows unsupervised hierarchical clustering of specimens based on the top 50 statistically significant DEGs separates specimens by migratory potential and by patient.

FIG. 15 is a table showing a summary of the demographic, tumor, surgical and clinical characteristics of the retrospective GBM patient cohort. Data are presented for either the entire population (n=28), or as categorized based on short- (<14.6 months, n=19) versus long-term survival (>14.6 months, n=9).

FIG. 16 is a table showing coefficients of logistic regression determined and used for the computation of composite MAqCI score.

FIG. 17 is a table showing individual numerical values behind the heat map classification presented in FIG. 3F.

FIG. 18 is a table showing Pearson correlation coefficients (R) for experimental variables and principal components (PC) of RNA-sequencing data of highly motile versus unsorted bulk GBM cells from two patients (GBM897 and 965).

FIG. 19 is a table showing GOBP analysis of DEGs in highly motile cells compared to the unsorted bulk population from two patients (GBM965 and 897). N is the total number of genes; B is the total number of genes associated with a specific GO term; n is the number of genes in the top of the user’s input list or in the target set when appropriate; and b is the number of genes in the intersection. Enrichment = (b/n) / (B/N).

FIG. 20 is a table showing upregulated DEGs whose gene expression patterns in highly motile GBM cells match those of GBM patients with reduced overall survival. Patients were stratified by median expression of each gene based on z-score. Hazard ratio is calculated for patients with high expression versus low expression of a gene. Three genes whose expression pattern in patients with worse OS did not match regulation in highly motile cells: UPB1, CARD10 and TCP1.

FIGS. 21A-B shows higher expression of HMOX1, NGO1 and PGK1 metabolic DEGSs results in reduced overall survival in breast and GBM datasets. FIG. 21A shows the probability of survival from the screening of HMOX1, NGO1 and PGK1 metabolic genes in breast cancer database. FIG. 21B shows the probability of survival from the screening of HMOX1, NGO1 and PGK1 metabolic genes in GBM database.

FIGS. 22A-B shows higher expression of VEGFA, ADM and HPCAL1 signaling related DEGSs results in reduced overall survival in breast and GBM datasets. FIG. 22A shows the probability of survival from the screening of VEGFA, ADM and HPCAL1 signaling genes in breast cancer database. FIG. 22B shows the probability of survival from the screening of VEGFA, ADM and HPCAL1 signaling genes in GBM database.

FIGS. 23A-B shows higher expression of PGK1, NQO1, HMOX1, VEGFA, ADM, HPCAL1, PLK3, FOSL1, and PLAU related DEGSs results in reduced overall survival in breast and GBM datasets. FIG. 23A shows the probability of survival from the screening of PGK1, NQO1, HMOX1, VEGFA, ADM, HPCAL1, PLK3, FOSL1, and PLA U genes in breast cancer database. FIG. 23B shows the probability of survival from the screening of PGK1, NQO1, HMOX1, VEGFA, ADM, HPCAL1, PLK3, FOSL1, and PLAU genes in GBM database.

FIG. 24 shows that paroxetine decreases the percent of migratory cells to the level of non-metastatic cells in metastatic MDA-MB-231 breast cancer cells.

### DETAILED DESCRIPTION

The present disclosure can be understood more readily by reference to the following detailed description of the invention, the figures and the examples included herein.

Before the present methods and gene expression panels are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed. As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “comprising″” can include the aspects “consisting of” and “consisting essentially of.”

As used herein, the term “normal” refers to an individual, a sample or a subject that does not have glioblastoma or breast cancer or does not have an increased susceptibility of developing glioblastoma or breast cancer.

As used herein, the term “susceptibility” refers to the likelihood of a subject being clinically diagnosed with a disease. For example, a human subject with an increased susceptibility for glioblastoma can refer to a human subject with an increased likelihood of a subject being clinically diagnosed with glioblastoma. Likewise, a human subject with an increased susceptibility for breast cancer can refer to a human subject with an increased likelihood of a subject being clinically diagnosed with breast cancer.

As used herein, the term “polypeptide” refers to any peptide, oligopeptide, polypeptide, gene product, expression product, or protein. A polypeptide is comprised of consecutive amino acids. The term “polypeptide” encompasses naturally occurring or synthetic molecules. As used herein, the term “amino acid sequence” refers to a list of abbreviations, letters, characters or words representing amino acid residues. In some aspects, the expression product or gene product can be a protein encoded by the gene.

As used herein, the term “gene” refers to a region of DNA encoding a functional RNA or protein. “Functional RNA” refers to an RNA molecule that is not translated into a protein. Generally, the gene symbol is indicated by using italicized styling while the protein symbol is indicated by using non-italicized styling.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

By “specifically binds” is meant that an antibody recognizes and physically interacts with its cognate antigen and does not significantly recognize and interact with other antigens; such an antibody may be a polyclonal antibody or a monoclonal antibody, which are generated by techniques that are well known in the art.

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By “specifically hybridizes” is meant that a probe, primer, or oligonucleotide recognizes and physically interacts (that is, base-pairs) with a substantially complementary nucleic acid under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8X SSC, 0.2 M Tris-Cl, pH 7.6, 1X Denhardt’s solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, NY, 1998).

“Inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100 percent, or any amount of reduction in between as compared to native or control levels. In some aspects, the inhibition or reduction is 10-20, 20-30, 30-40, 40-50, 50-60, 60-70, 70-80, 80-90, or 90-100 percent as compared to native or control levels. In some aspects, the inhibition or reduction is 0-25, 25-50, 50-75, or 75-100 percent as compared to native or control levels.

The term “contacting” as used herein refers to bringing an antibody, a capture agent, compound or test agent and a cell, target receptor, antigen, peptide, protein, or other biological entity together in such a manner that the an antibody, a capture agent, compound or test agent can interact with the cell, target receptor, antigen, peptide, protein, or other biological entity (e.g., by interacting with the cell, target receptor, antigen, peptide, protein, or other biological entity).

As used herein, the term “level” refers to the amount of a target molecule (e.g., a gene or a protein) in a sample, e.g., a sample from a subject. The amount of the target molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compares to a control (i.e., a non-affected or healthy subject or a sample from a non-affected or healthy subject) or a sham or an untreated sample) or comparing two or more samples obtained from the same subject but at different time points.

The phrase “at least” preceding a series of elements is to be understood to refer to every element in the series. For example, “at least one” includes one, two, three, four or more.

As used herein, “effective amount” of a compound is meant to mean a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

As used herein, “treat” is meant to mean administer a compound or molecule of the invention to a subject, such as a human or other mammal (for example, an animal model), that has a glioblastoma or breast cancer, in order to prevent or delay a worsening of the effects of the disease or condition, or to partially or fully reverse the effects of the disease.

As used herein, “prevent” is meant to mean minimize the chance that a subject who has an increased susceptibility for developing cancer will develop cancer. As further used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “reference,” “reference expression,” “reference sample,” “reference value,” “control,” “control sample” and the like, when used in the context of a sample or level or amount of cell surface marker-expressing cells refers to a reference standard wherein the reference is expressed at a constant level and is unaffected by the experimental conditions, and is indicative of the level in a sample of a predetermined disease status (e.g., not suffering from a glioblastoma or breast cancer). The reference value can be a predetermined standard value or a range of predetermined standard values, representing no illness, or a predetermined type or severity of illness.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with glioblastoma” or “diagnosed with breast cancer” means having been subjected to an examination by a person of skill, for example, a physician, and found to have a condition that can be diagnosed or treated by a compound or composition disclosed herein.

As used herein, the term “prognosis” defines a forecast as to the probable outcome of a disease (e.g., glioblastoma or breast cancer), the prospect as to recovery from a disease, or the potential recurrence of a disease as indicated by the nature and symptoms of the case.

As used herein, the term “cell” or “cells” refers to a single cell as well as a plurality or population of cells.

As used herein, the term “marker” or “biomarker” or “cell-surface marker” refers to a biological molecule, e.g., a nucleic acid, peptide, protein, hormone, etc., whose presence or concentration can be detected and correlated with a known condition, such as a disease state. In some aspects, the disclosed markers or biomarkers can also be cell-surface markers. In some aspects, the disclosed markers or biomarkers are not expressed on the cell surface. For example, in some aspects, the biomarkers PLAU and ARL4C can be expressed extracellularly. In some aspects, PLAU and ARL4C can be expressed in cytoplasm.

As used herein, the term “predict” or “prediction” can refer to the likelihood that a subject will have a particular clinical outcome, whether positive or negative. For instance, the term “prediction” may refer to the likelihood that a subject will respond either favorably or unfavorably to a drug (or therapy or therapeutic agent) or set of drugs, and also the extent of those responses, or that a patient will survive, following surgical removal of the primary tumor and/or therapy for a certain period of time without glioblastoma or breast cancer recurrence. The predictive methods of the present invention can be used clinically to make treatment decisions by choosing the most appropriate treatment modalities for any particular subject. The predictive methods of the present invention are valuable tools in predicting if a patient is likely to respond favorably to a treatment regimen, such as surgical intervention, therapy with a given drug or drug combination, and/or radiation therapy, or whether long-term survival of the patient, following surgery and/or termination of therapy is likely. The predictive methods of the present invention can be used clinically to make treatment decisions by choosing the most appropriate treatment modalities for any particular subject. The predictive methods of the present invention are valuable tools in predicting if a subject is likely to respond favorably to a treatment regimen, such as a chemotherapy, an immunotherapy, or radiation. In some aspects, the methods disclosed herein can be used to predict whether a subject has an increased risk of recurrence of glioblastoma or breast cancer. In some aspects, the methods disclosed herein can be used to predict whether a subject has a short survival after being diagnosed with glioblastoma. In some aspects, the methods disclosed herein can be used to predict whether a subject has a short survival after being diagnosed with breast cancer.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Glioblastoma (GBM) is the most common and aggressive type of primary brain cancer in adults, accounting for about 15-20% of brain malignancies. Owing to its highly proliferative and infiltrative nature, the median survival of GBM patients is approximately 14.6 months, with less than 5% of patients surviving past 5 years. GBMs invade locally into the surrounding brain parenchyma and frequently spread to the contralateral hemisphere through the corpus callosum, thereby confounding local therapy and rendering gross total resection nearly impossible (Stupp, R. et al., N Engl J Med 352, 987-996 (2005)), Chaichana, K. L. et al, Neuro Oncol 16, 113-122 (2014)), and Shah, S. R. et al., bioRxiv, 602052, doi:10.1101/602052 (2019)). As a result, despite aggressive radical surgical resection coupled with concurrent chemo- and radio-therapy, GBMs remain incurable and recur frequently (Chaichana, K. L. et al., J Neurosurg 118, 812-820 (2013)).

To date, there is a lack of testing platforms that can effectively predict GBM outcomes in a patient-specific manner. While certain demographic (e.g. age), tumor (e.g. tumor locations, cytologic and histologic compositions) and clinical parameters (e.g. Karnofsky Performance Score) have demonstrated some prognostic values for survival correlation, they are often confounded by patient comorbidities, and thus rarely affect GBM treatment decision. Recent advancements in proteomics and genomics have identified certain molecular markers, such as O6-methylguanine DNA-methyltransferase (MGMT) promoter methylation and isocitrate dehydrogenase 1 (IDH1) mutation status, as independent prognostic factors for gliomas. MGMT promoter methylation has been shown to be associated with longer overall survival and enhanced sensitivity to therapy. However, inter-and intra-tumoral heterogeneity coupled with the lack of standardization and reproducibility of MGMT methylation status classification have prevented its widespread use in the clinic. IDH1 mutation status has emerged as a leading prognostic marker for gliomas. Specifically, low-grade glioma patients harboring the mutant form of IDH1 have improved prognosis and median survival compared to those expressing the wild type IDH1.

Yet, the prognostic power of IDH1 mutation status on primary GBMs remains limited as IDH1 mutations are often associated with lower grades diffuse gliomas (Grade II and III) and with secondary GBMs. Finally, the use of laborious and time-consuming ex vivo expansion of cancer cells in murine xenograft model for phenotypic testing is impractical for informing patient care given the short survival span of GBM patients.

Cell population-based molecular analysis techniques often overlook the diversity of cancer cells and suffer from the inability to discern inter- and intra-tumoral heterogeneity that may contribute to the aggressiveness of GBMs. While high-throughput single-cell genomic and proteomic analyses could potentially ameliorate the problem of tumor heterogeneity, these techniques require sophisticated and expensive equipment and facilities, rendering their widespread application currently infeasible in most clinical settings. Importantly, the aggressiveness of cancers is frequently a result of an amalgamation of multiple distinct combinations of genetic and proteomic alterations, which cannot be predicted accurately by just one or two molecular markers and might be difficult to decipher. The heterogeneous and complex nature of GBMs therefore necessitates the development of a more direct, faster, inexpensive, high-throughput and unbiased in vitro testing platform for GBM prognosis capable of dissecting the heterogeneity among the cancer cells derived from individual patients. It is known that highly metastatic subpopulations of cancer cells have enhanced motility and proliferation rates that are linked to the aggressiveness and invasiveness of the cancer. Along these lines, a Microfluidic Assay for Quantification of Cell Invasion (MAqCI) can be used to measure both the migratory and proliferative potentials of breast cancer cells for the purpose of assessing their metastatic propensity and screening of potential antimetastatic therapeutics.

As described herein, it was tested whether MAqCI could be leveraged to identify a subpopulation of migratory and proliferative cells within a GBM patient-derived specimen whose prevalence would serve as a metric for predicting the aggressiveness of the disease and clinical prognosis.

### Methods

Disclosed herein, are methods for identifying a subject with an increased risk of short survival and/or recurrence of glioblastoma. The method comprises the steps of, in any order, a) obtaining a brain tissue sample or having obtained a brain tissue sample from a subject; b) determining gene expression levels of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample from the subject; and c) optionally comparing the level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 to a predetermined reference level identifying a subject with an increased risk of short survival and/or recurrence of glioblastoma when the level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample is determined to be higher than a predetermined reference level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample. In some aspects, steps a) and b) can be repeated.

In some aspects, the methods for identifying a subject with an increased risk of short survival and/or recurrence of glioblastoma can comprise the steps of, in any order, a) obtaining a brain tissue sample or having obtained a brain tissue sample from a subject; b) determining gene expression levels of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample from the subject; and c) optionally comparing the level of gene expression of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 to a predetermined reference level identifying a subject with an increased risk of short survival and/or recurrence of glioblastoma when the level of gene expression of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample is determined to be higher than a predetermined reference level of gene expression of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample. In some aspects, steps a) and b) can be repeated.

Disclosed herein, are methods for identifying a subject with an increased risk of short survival and/or recurrence of glioblastoma. The method comprises the steps of, in any order, a) obtaining a brain tissue sample or having obtained a brain tissue sample from a subject; b) determining gene expression levels of at least ten of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample from the subject; and c) optionally comparing the level of gene expression of at least ten of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 to a predetermined reference level identifying a subject with an increased risk of short survival and/or recurrence of glioblastoma when the level of gene expression of the at least ten of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample is determined to be higher than a predetermined reference level of gene expression of the at least ten of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample. In some aspects, steps a) and b) can be repeated.

Also disclosed herein are methods comprising determining gene expression levels of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in a brain sample. In some aspects, the methods can comprise determining gene expression levels of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in a brain sample.

Disclosed herein, are methods for identifying a subject with an increased risk of short survival and/or recurrence of breast cancer. The method comprises the steps of, in any order, a) obtaining a breast tissue sample or having obtained a breast tissue sample from a subject; b) determining gene expression levels of PGK1, NQO1, HMOX1, VEGFA, ADM, HPCAL1, PLK3, FOSL1, and PLAU in the sample from the subject; and c) optionally comparing the level of gene expression of PGK1, NQO1, HMOX1, VEGFA, ADM, HPCAL1, PLK3, FOSL1, and PLAU to a predetermined reference level identifying a subject with an increased risk of short survival and/or recurrence of glioblastoma when the level of gene expression of PGK1, NQO1, HMOX1, VEGFA, ADM, HPCAL1, PLK3, FOSL1, and PLAU in the sample is determined to be higher than a predetermined reference level of gene expression of PGK1, NQO1, HMOX1, VEGFA, ADM, HPCAL1, PLK3, FOSL1, and PLAU in the sample. In some aspects, steps a) and b) can be repeated.

In some aspects, the methods for identifying a subject with an increased risk of short survival and/or recurrence of breast cancer can comprise the steps of, in any order, a) obtaining a breast tissue sample or having obtained a breast tissue sample from a subject; b) determining gene expression levels of one or more of PGK1, NQO1, HMOX1, VEGFA, ADM, HPCAL1, PLK3, FOSL1, and PLAUin the sample from the subject; and c) optionally comparing the level of gene expression of one or more of PGK1, NQO1, HMOX1, VEGFA, ADM, HPCAL1, PLK3, FOSL1, and PLAUto a predetermined reference level identifying a subject with an increased risk of short survival and/or recurrence of glioblastoma when the level of gene expression of one or more of PGK1, NQO1, HMOX1, VEGFA, ADM, HPCAL1, PLK3, FOSL1, and PLAU in the sample is determined to be higher than a predetermined reference level of gene expression of one or more of PGK1, NQO1, HMOX1, VEGFA, ADM, HPCAL1, PLK3, FOSL1, and PLAU in the sample. In some aspects, steps a) and b) can be repeated.

Disclosed herein, are methods for identifying a subject with an increased risk of short survival and/or recurrence of breast cancer. The method comprises the steps of, in any order, a) obtaining a breast tissue sample or having obtained a breast tissue sample from a subject; b) determining gene expression levels of at least two, three, four, five, six, seven, eight, or nine of PGK1, NQO1, HMOX1, VEGFA, ADM, HPCAL1, PLK3, FOSL1, and PLAU in the sample from the subject; and c) optionally comparing the level of gene expression of at least two, three, four, five, six, seven, eight, or nine of PGK1, NQO1, HMOX1, VEGFA, ADM, HPCAL1, PLK3, FOSL1, and PLAU to a predetermined reference level identifying a subject with an increased risk of short survival and/or recurrence of breast cancer when the level of gene expression of the at least two, three, four, five, six, seven, eight, or nine of PGK1, NQO1, HMOX1, VEGFA, ADM, HPCAL1, PLK3, FOSL1, and PLAU in the sample is determined to be higher than a predetermined reference level of gene expression of the at least two, three, four, five, six, seven, eight, or nine of PGK1, NQO1, HMOX1, VEGFA, ADM, HPCAL1, PLK3, FOSL1, and PLAU in the sample. In some aspects, steps a) and b) can be repeated.

Also disclosed herein are methods comprising determining gene expression levels of PGK1, NQO1, HMOX1, VEGFA, ADM, HPCAL1, PLK3, FOSL1, and PLAU in a brain sample. In some aspects, the methods can comprise determining gene expression levels of one or more of PGK1, NQO1, HMOX1, VEGFA, ADM, HPCAL1, PLK3, FOSL1, and PLAU in a brain sample. In some aspects, the methods can comprise determining gene expression levels of two or more of PGK1, NQO1, HMOX1, VEGFA, ADM, HPCAL1, PLK3, FOSL1, and PLAU in a brain sample. In some aspects, the methods can comprise determining gene expression levels of three or more of PGK1, NQO1, HMOX1, VEGFA, ADM, HPCAL1, PLK3, FOSL1, and PLAU in a brain sample. In some aspects, the methods can comprise determining gene expression levels of four or more of PGK1, NQO1, HMOX1, VEGFA, ADM, HPCAL1, PLK3, FOSL1, and PLAU in a brain sample. In some aspects, the methods can comprise determining gene expression levels of five or more of PGK1, NQO1, HMOX1, VEGFA, ADM, HPCAL1, PLK3, FOSL1, and PLAU in a brain sample. In some aspects, the methods can comprise determining gene expression levels of six or more of PGK1, NQO1, HMOX1, VEGFA, ADM, HPCAL1, PLK3, FOSL1, and PLAU in a brain sample. In some aspects, the methods can comprise determining gene expression levels of seven or more of PGK1, NQO1, HMOX1, VEGFA, ADM, HPCAL1, PLK3, FOSL1, and PLAU in a brain sample. In some aspects, the methods can comprise determining gene expression levels of eight or more of PGK1, NQO1, HMOX1, VEGFA, ADM, HPCAL1, PLK3, FOSL1, and PLAU in a brain sample. In some aspects, the methods can comprise determining gene expression levels of nine of PGK1, NQO1, HMOX1, VEGFA, ADM, HPCAL1, PLK3, FOSL1, and PLAU in a brain sample.

In some aspects, the methods can further comprise administering a therapeutic to the subject identified to have an increased risk of short-survival and/or recurrence of glioblastoma. In some aspects, the therapeutic can be chemotherapy, radiation therapy, or therapy targeted to specific pathways known to be important in glioblastoma or the immune system. In some aspects, the therapeutic can be chemotherapy, radiation therapy, or therapy targeted to specific pathways known to be important in breast cancer or the immune system. In some aspects, the therapeutic can be radiation therapy and chemotherapy. In some aspects, the chemotherapy can be temozolomide. In some aspects, the therapeutic can be Avastin® (bevacizumab). In some aspects, the therapeutic can be a checkpoint inhibitor. In some aspects, the checkpoint inhibitor can be pembrolizumab. In some aspects, the therapeutic can be a checkpoint regulator. In some aspects, the checkpoint regulator can be flavopiridol, palbociclib, dinacliclib, roscovittine, milciclib, or purvalanol A. In some aspects, the therapeutic agent can be a serotonin reuptake inhibitor (SSRI). In some aspects, the SSRI can be paroxetine, citalopram, escitalopram, fluoxetine, fluvoxamine, sertraline, or vilazodone. In some aspects, the therapeutic can be a typical antipsychotic drug. Examples of typical antipsychotic drugs include but are not limited to haloperidol, trifluoperazine, fluphenazine, thioridazine, perphenazine and chlorpromazine. In some aspects, the therapeutic can be an atypical antipsychotic drug. Examples of atypical antipsychotic drugs include but are not limited to olanzapine, clozapine, asenapine, lurasidone, quetiapine, risperidone, and aripiprazole. In some aspects, the therapeutic can be a tricyclic antidepressant. Examples of tricyclic antidepressants include but are not limited to amitriptyline, imipramine, clopiramine, doxepin, and amoxapine. In some aspects, the therapeutic can be a sedative hypnotic. Examples of sedative hypnotics can be benzodiazepines, including but not limited to diazepam, lorazepam, triazolam, temazepam, oxazepam and midazolam. In some aspects, the therapeutic can be an antiepileptic drug. Examples of antiepileptic drugs include but are not limited to sodium valproate, carbamazepine, and levetiracetam. In some aspects, the therapeutic agent can be disulfiram. In some aspects, the therapeutic agent can be a microtubule inhibitor. In some aspects, the microtubule inhibitor can be mebendazole or vincristine. In some aspects, the therapeutic agent can be clomifene. In some aspects, the therapeutic agent can be metformin or phenformin. In some aspects, the therapeutic agent can be repaglinides. In some aspects, the therapeutic can be an EGFR inhibitor. Examples of EGFR inhibitors include but are not limited to erlotinib, gefitini, nimotuzumab and cetuximab. In some aspects, the therapeutic can be a statin. Examples of statins include but are not limited to lovastatin, pravastatin, rosuvastatin and simvastatin. In some aspects, the therapeutic can be administered after maximal surgical resection.

In some aspects, the methods can further comprise administering a therapeutic to the subject identified to have an increased risk of short-survival and/or recurrence of breast cancer. In some aspects, the therapeutic can be chemotherapy, radiation therapy, or therapy targeted to specific pathways known to be important in breast cancer or the immune system. In some aspects, the therapeutic can be chemotherapy, radiation therapy, or therapy targeted to specific pathways known to be important in breast cancer or the immune system. In some aspects, the therapeutic can be radiation therapy and chemotherapy. In some aspects, the chemotherapy can be an anthracycline (e.g., doxorubicin (Adriamycin) and epirubicin (Ellence), a taxane (e.g., paclitaxel (Taxol) and docetaxel (Taxotere), 5-fluorouracil (5-FU) or capecitabine, cyclophosphamide (Cytoxan) and carboplatin (Paraplatin). In some aspects, the therapeutic can be Avastin® (bevacizumab). In some aspects, the therapeutic can be any combination of any of the chemotherapeutics described herein. In some aspects, the therapeutic can be a targeted therapy for breast cancer subtypes (e.g., hormone therapy for estrogen receptor positive breast cancer or progesterone receptor positive breast cancer or Herceptin for HER2 positive breast cancer). In some aspects, the therapeutic can be a checkpoint inhibitor. In some aspects, the checkpoint inhibitor can be pembrolizumab. In some aspects, the therapeutic agent can be a serotonin reuptake inhibitor (SSRI). In some aspects, the SSRI can be paroxetine, citalopram, escitalopram, fluoxetine, fluvoxamine, sertraline, or vilazodone. In some aspects, the therapeutic can be a checkpoint regulator. In some aspects, the checkpoint regulator can be flavopiridol, palbociclib, dinacliclib, roscovittine, milciclib, or purvalanol A. In some aspects, the therapeutic can be a typical antipsychotic drug. Examples of typical antipsychotic drugs include but are not limited to haloperidol, trifluoperazine, fluphenazine, thioridazine, perphenazine and chlorpromazine. In some aspects, the therapeutic can be an atypical antipsychotic drug. Examples of atypical antipsychotic drugs include but are not limited to olanzapine, clozapine, asenapine, lurasidone, quetiapine, risperidone, and aripiprazole. In some aspects, the therapeutic can be a tricyclic antidepressant. Examples of tricyclic antidepressants include but are not limited to amitriptyline, imipramine, clopiramine, doxepin, and amoxapine. In some aspects, the therapeutic can be a sedative hypnotic. Examples of sedative hypnotics can be benzodiazepines, including but not limited to diazepam, lorazepam, triazolam, temazepam, oxazepam and midazolam. In some aspects, the therapeutic can be an antiepileptic drug. Examples of antiepileptic drugs include but are not limited to sodium valproate, carbamazepine, and levetiracetam. In some aspects, the therapeutic agent can be disulfiram. In some aspects, the therapeutic agent can be a microtubule inhibitor. In some aspects, the microtubule inhibitor can be mebendazole or vincristine. In some aspects, the therapeutic agent can be clomifene. In some aspects, the therapeutic agent can be metformin or phenformin. In some aspects, the therapeutic agent can be repaglinides. In some aspects, the therapeutic can be an EGFR inhibitor. Examples of EGFR inhibitors include but are not limited to erlotinib, gefitini, nimotuzumab and cetuximab. In some aspects, the therapeutic can be a statin. Examples of statins include but are not limited to lovastatin, pravastatin, rosuvastatin and simvastatin.In some aspects, the therapeutic can be administered after maximal surgical resection.

The method described herein can also be carried out with one or more diagnostic tests (e.g., nucleic acid assay or protein assay).

In some aspects, the methods can further comprise determining the gene expression level of Ki67 in the sample from the subject. Ki-67 is a protein in cells that increases as they prepare to divide into new cells. For example, in some aspects, the Ki-67 level (gene or protein) can correlate to faster cancer growth (e.g., proliferation). A staining process can measure the percentage of tumor cells that are positive for Ki-67. In some aspects, a high level of Ki67 can indicate an aggressive cancer. In some aspects, a Ki67 index can be determined. The Ki67 index can be determined by counting the total number of Ki67 positive tumor cells and dividing by the total number of tumor cells, and multiplying that value by 100. In some aspects, a Ki67 index of less than 6% can be considered low, and indicate that the cancer is non-aggressive or is less aggressive. In some aspects, a Ki67 index of 6-10% can be considered intermediate, and indicate that the cancer is less aggressive. In some aspects, a Ki67 index of more than 10% can be considered high, and indicate that the cancer is aggressive. In some aspects, the cancer can be GBM or breast cancer.

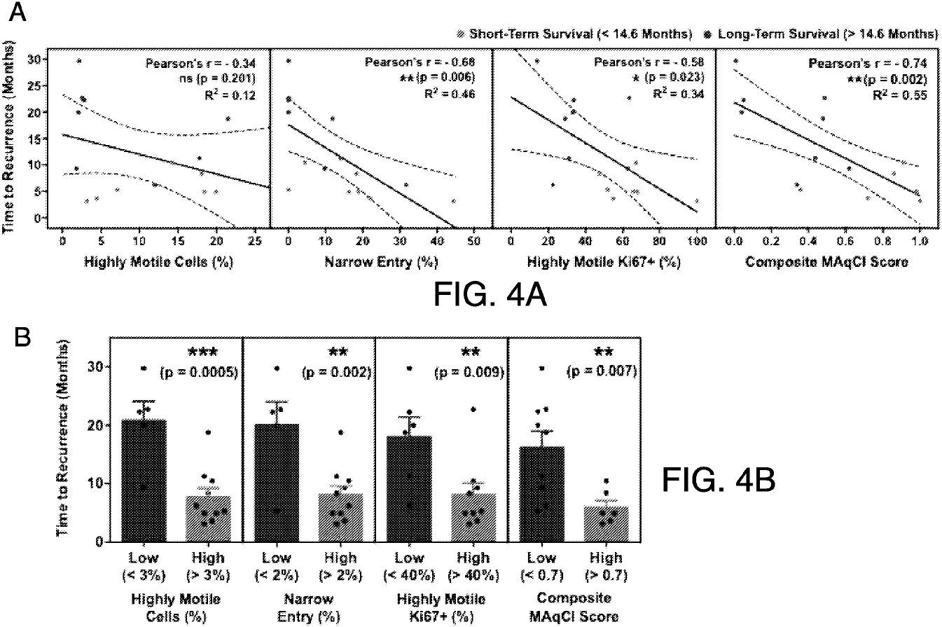

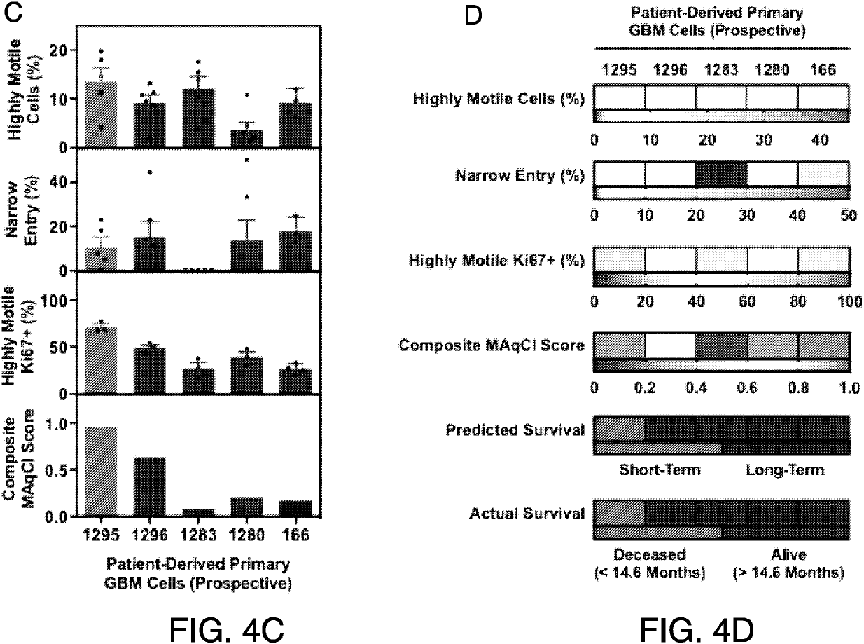

In some aspects and as described herein, in samples from subjects with GBM, the higher the percentage of migratory cells that stain positive for Ki67, the shorter the survival time (see, FIG. 4A, panel 3). In some aspects, the percentage of Ki67 positive tumor cells that are considered highly motile cells that is used to categorize glioblastoma or breast cancer subjects into long-term survival is around 45% or lower. In some aspects, the percentage of Ki67 positive tumor cells that are considered highly motile cells that is used to categorize glioblastoma or breast cancer subjects into short-term survival is around 45% or higher. In some aspects and as described herein, in samples from subjects with glioblastoma or breast cancer, the higher the percentage of migratory cells that stain positive for Ki67, the shorter the survival time. This Ki67 value is obtained by systematically varying the discriminating threshold and comparing the values of prediction performance (i.e., sensitivity, specificity, PPV, NPV and accuracy) at which MAqCI correctly classifies subjects into based on their survival outcomes in a retrospective cohort.

In some aspects, the methods can further comprise quantifying cell invasion of cells from the sample using a microfluidic assay. In some aspects, the methods can further comprise determining the invasiveness of a cell or a population of cells from the brain or breast tissue sample. In some aspects, the cell or the population of cells from the brain or breast tissue sample can be incubated and imaged in an integrative microfluidic apparatus. In some aspects, the methods can further comprise determining whether the cells or population of cells in the sample are invasive when the cell or population of cells migrates through the migratory channel of the apparatus and to the bifurcation point of the channel. In some aspects, an integrative microfluidic apparatus as described in PCT/US2016/064725 and PCT/US2014/046639 can be used. PCT/US2016/064725 and PCT/US2014/046639 are hereby incorporated by reference in their entirety. In some aspects, an integrative microfluidic apparatus as described in Yankaskas et al., Nat Biomed Eng., 2019 Jun; 3(6):452-465 can also be used. Yankaskas et al., Nat Biomed Eng., 2019 Jun; 3(6):452-465 is hereby incorporated by reference in its entirety.

Also disclosed herein are methods of treating a subject with an increased risk of recurrence of glioblastoma. In some aspects, the methods can comprise a) obtaining a brain tissue sample or having obtained a brain tissue sample from a subject; b) determining gene expression levels of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample from the subject; c) identifying that the subject to have an increased risk of recurrence of glioblastoma when the level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample is determined to be higher than a predetermined reference level of gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1; and d) administering a suitable cancer therapeutic to the subject having an increased risk of recurrence of glioblastoma. In some aspects, the methods can comprise a) obtaining a brain tissue sample or having obtained a brain tissue sample from a subject; b) determining gene expression levels of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample from the subject; c) identifying that the subject to have an increased risk of recurrence of glioblastoma when the level of gene expression of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample is determined to be higher than a predetermined reference level of gene expression of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1; and d) administering a suitable cancer therapeutic to the subject having an increased risk of recurrence of glioblastoma. In some aspects, the suitable cancer therapeutic can be chemotherapy, radiation therapy, or therapy targeted to specific pathways known to be important in glioblastoma or the immune system. In some aspects, the suitable cancer therapeutic can be radiation therapy and chemotherapy. In some aspects, the chemotherapy can be temozolomide. In some aspects, the suitable cancer therapeutic can be Avastin® (bevacizumab). In some aspects, the therapeutic can be a checkpoint inhibitor. In some aspects, the checkpoint inhibitor can be pembrolizumab. In some aspects, the therapeutic can be a checkpoint regulator. In some aspects, the checkpoint regulator can be flavopiridol, palbociclib, dinacliclib, roscovittine, milciclib, or purvalanol A. In some aspects, the therapeutic agent can be a serotonin reuptake inhibitor (SSRI). In some aspects, the SSRI can be paroxetine, citalopram, escitalopram, fluoxetine, fluvoxamine, sertraline, or vilazodone. In some aspects, the therapeutic can be a typical antipsychotic drug. Examples of typical antipsychotic drugs include but are not limited to haloperidol, trifluoperazine, fluphenazine, thioridazine, perphenazine and chlorpromazine. In some aspects, the therapeutic can be a atypical antipsychotic drug. Examples of atypical antipsychotic drugs include but are not limited to olanzapine, clozapine, asenapine, lurasidone, quetiapine, risperidone, and aripiprazole. In some aspects, the therapeutic can be a tricyclic antidepressant. Examples of tricyclic antidepressants include but are not limited to amitriptyline, imipramine, clopiramine, doxepin, and amoxapine. In some aspects, the therapeutic can be a sedative hypnotic. Examples of sedative hypnotics can be benzodiazepines, including but not limited to diazepam, lorazepam, triazolam, temazepam, oxazepam and midazolam. In some aspects, the therapeutic can be an antiepileptic drug. Examples of antiepileptic drugs include but are not limited to sodium valproate, carbamazepine, and levetiracetam. In some aspects, the therapeutic agent can be disulfiram. In some aspects, the therapeutic agent can be a microtubule inhibitor. In some aspects, the microtubule inhibitor can be mebendazole or vincristine. In some aspects, the therapeutic agent can be clomifene. In some aspects, the therapeutic agent can be metformin or phenformin. In some aspects, the therapeutic agent can be repaglinides. In some aspects, the therapeutic can be an EGFR inhibitor. Examples of EGFR inhibitors include but are not limited to erlotinib, gefitini, nimotuzumab and cetuximab. In some aspects, the therapeutic can be a statin. Examples of statins include but are not limited to lovastatin, pravastatin, rosuvastatin and simvastatin. In some aspects, the therapeutic can be administered after maximal surgical resection.

Further disclosed herein are methods of treating a subject with an increased risk of recurrence of breast cancer. In some aspects, the methods can comprise a) obtaining a breast tissue sample or having obtained a breast tissue sample from a subject; b) determining gene expression levels of PGK1, NQO1, HMOX1, VEGFA, ADM, HPCAL1, PLK3, FOSL1, and PLAU in the sample from the subject; c) identifying that the subject to have an increased risk of recurrence of breast cancer when the level of gene expression of PGK1, NQO1, HMOX1, VEGFA, ADM, HPCAL1, PLK3, FOSL1, and PLAU in the sample is determined to be higher than a predetermined reference level of gene expression PGK1, NQO1, HMOX1, VEGFA, ADM, HPCAL1, PLK3, FOSL1, and PLAU; and d) administering a suitable cancer therapeutic to the subject having an increased risk of recurrence of breast cancer. In some aspects, the methods can comprise a) obtaining a breast tissue sample or having obtained a breast tissue sample from a subject; b) determining gene expression levels of one or more of PGK1, NQO1, HMOX1, VEGFA, ADM, HPCAL1, PLK3, FOSL1, and PLAU in the sample from the subject; c) identifying that the subject to have an increased risk of recurrence of breast cancer when the level of gene expression of one or more of PGK1, NQO1, HMOX1, VEGFA, ADM, HPCAL1, PLK3, FOSL1, and PLAU in the sample is determined to be higher than a predetermined reference level of gene expression one or more of PGK1, NQO1, HMOX1, VEGFA, ADM, HPCAL1, PLK3, FOSL1, and PLAU; and d) administering a suitable cancer therapeutic to the subject having an increased risk of recurrence of breast cancer. In some aspects, the suitable cancer therapeutic can be chemotherapy, radiation therapy, or therapy targeted to specific pathways known to be important in glioblastoma or the immune system. In some aspects, the suitable cancer therapeutic can be radiation therapy and chemotherapy. In some aspects, the chemotherapy can be an anthracycline (e.g., doxorubicin (Adriamycin) and epirubicin (Ellence), a taxane (e.g., paclitaxel (Taxol) and docetaxel (Taxotere), 5-fluorouracil (5-FU) or capecitabine, cyclophosphamide (Cytoxan) and carboplatin (Paraplatin). In some aspects, the suitable cancer therapeutic can be Avastin® (bevacizumab). In some aspects, the therapeutic can be any combination of any of the chemotherapeutics described herein. In some aspects, the therapeutic can be a targeted therapy for breast cancer subtypes (e.g., hormone therapy for estrogen receptor positive breast cancer or progesterone receptor positive breast cancer or Herceptin for HER2 positive breast cancer). In some aspects, the therapeutic can be a checkpoint inhibitor. In some aspects, the checkpoint inhibitor can be pembrolizumab. In some aspects, the therapeutic agent can be a serotonin reuptake inhibitor (SSRI). In some aspects, the SSRI can be paroxetine, citalopram, escitalopram, fluoxetine, fluvoxamine, sertraline, or vilazodone. In some aspects, the therapeutic can be a checkpoint regulator. In some aspects, the checkpoint regulator can be flavopiridol, palbociclib, dinacliclib, roscovittine, milciclib, or purvalanol A. In some aspects, the therapeutic can be a typical antipsychotic drug. Examples of typical antipsychotic drugs include but are not limited to haloperidol, trifluoperazine, fluphenazine, thioridazine, perphenazine and chlorpromazine. In some aspects, the therapeutic can be an atypical antipsychotic drug. Examples of atypical antipsychotic drugs include but are not limited to olanzapine, clozapine, asenapine, lurasidone, quetiapine, risperidone, and aripiprazole. In some aspects, the therapeutic can be a tricyclic antidepressant. Examples of tricyclic antidepressants include but are not limited to amitriptyline, imipramine, clopiramine, doxepin, and amoxapine. In some aspects, the therapeutic can be a sedative hypnotic. Examples of sedative hypnotics can be benzodiazepines, including but not limited to diazepam, lorazepam, triazolam, temazepam, oxazepam and midazolam. In some aspects, the therapeutic can be an antiepileptic drug. Examples of antiepileptic drugs include but are not limited to sodium valproate, carbamazepine, and levetiracetam. In some aspects, the therapeutic agent can be disulfiram. In some aspects, the therapeutic agent can be a microtubule inhibitor. In some aspects, the microtubule inhibitor can be mebendazole or vincristine. In some aspects, the therapeutic agent can be clomifene. In some aspects, the therapeutic agent can be metformin or phenformin. In some aspects, the therapeutic agent can be repaglinides. In some aspects, the therapeutic can be an EGFR inhibitor. Examples of EGFR inhibitors include but are not limited to erlotinib, gefitini, nimotuzumab and cetuximab. In some aspects, the therapeutic can be a statin. Examples of statins include but are not limited to lovastatin, pravastatin, rosuvastatin and simvastatin. In some aspects, the therapeutic can be administered after breast-conserving surgery.

Obtaining a tissue sample. Procedures for the extraction and collection of a sample of a subject’s brain or breast tissue can be done by methods known in the art. Brain and breast tissue obtained via biopsy is standard practice. Frozen tissue specimens can also be used. As noted above, tissue samples can be obtained from the subject’s resected brain tumor (e.g., after maximal surgical resection or using core needle biopsies) or from the subject’s breast tumor during or after breast-conserving surgery. The sample can be whole cells or cell organelles. Cells can be collected by scraping the tissue, processing the tissue sample to release individual cells or isolating the cells from a bodily fluid. The sample can be fresh tissue, dry tissue, cultured cells or tissue. The sample can be unfixed or fixed. Any part of the brain or breast can be obtained and assessed using the methods described herein.

In some aspects, the sample can be from a subject undergoing brain resection surgery. In some aspects, the subject has been diagnosed with glioblastoma. In some aspects, the methods described herein can be repeated in a subject identified with recurrent glioblastoma or in a subject identified with an increased risk of recurrent glioblastoma. In some aspects, the methods can further comprise selecting an interval for monitoring the subject for changes in the glioblastoma. In some aspects, magnetic resonance imaging can be used to monitor the subject at intervals for changes in the glioblastoma. In some aspects, the methods can further comprise selecting one or more tests for monitoring the subject at the selected interval for changes in the glioblastoma. In some aspects, the methods can further comprise providing a diagnosis to the subject from which the tissue sample was obtained. In some aspects, the methods can further comprise providing a prognosis to the subject from which the tissue sample was obtained. In some aspects, the prognosis can be survival time. In some aspects, the subject can be identified as having an increased risk of short survival. In some aspects, short survival can be 14.6 months or less. In some aspects, the subject can be identified as not having an increased risk of short survival. In some aspects, survival time can be 14.7 months or longer. In some aspects, the subject can be identified as having an increased risk of recurrence of glioblastoma.

In some aspects, the sample can be from a subject undergoing breast-conserving surgery. In some aspects, the breast-conserving surgery can be a lumpectomy, quadrantectomy, partial or segmental mastectomy. In some aspects, the subject has been diagnosed with breast cancer. In some aspects, the methods described herein can be repeated in a subject identified with recurrent breast cancer or in a subject identified with an increased risk of recurrent breast cancer. In some aspects, the methods can further comprise selecting an interval for monitoring the subject for changes in the breast cancer. In some aspects, a mammogram can be used to monitor the subject at intervals for changes in the breast cancer. In some aspects, the methods can further comprise selecting one or more tests for monitoring the subject at the selected interval for changes in the breast cancer. In some aspects, the methods can further comprise providing a diagnosis to the subject from which the tissue sample was obtained. In some aspects, the methods can further comprise providing a prognosis to the subject from which the tissue sample was obtained. In some aspects, the prognosis can be survival time. In some aspects, the subject can be identified as having an increased risk of short survival. In some aspects, short survival can be 30-55 months or less. In some aspects, the subject can be identified as not having an increased risk of short survival. In some aspects, survival time can be 56 months or longer. In some aspects, the subject can be identified as having an increased risk of recurrence of breast cancer.

In some aspects, the breast cancer can be ductal carcinoma in situ, invasive ductal carcinoma, inflammatory breast cancer or metastatic breast cancer.

Determining expression level. As used herein, the term “expression,” when used in the context of determining or detecting the expression or expression level of one or more genes, can refer to determining or detecting transcription of the gene (i.e., determining mRNA levels) and/or determining or detecting translation of the gene (e.g., determining or detecting the protein produced). To determine the expression level of a gene means to determine whether or not a gene is expressed, and if expressed, to what relative degree.

The expression level of one or more genes disclosed herein can be determined directly (e.g., immunoassays, mass spectrometry) or indirectly (e.g., determining the mRNA expression of a protein or peptide). Examples of mass spectrometry include ionization sources such as EI, CI, MALDI, ESI, and analysis such as Quad, ion trap, TOF, FT or combinations thereof, spectrometry, isotope ratio mass spectrometry (IRMS), thermal ionization mass spectrometry (TIMS), spark source mass spectrometry, Multiple Reaction Monitoring (MRM) or SRM. Any of these techniques can be carried out in combination with prefractionation or enrichment methods. Examples of immunoassays include immunoblots, Western blots, Enzyme linked Immunosorbant Assay (ELISA), Enzyme immunoassay (EIA), radioimmune assay. Immunoassay methods use antibodies for detection and determination of levels of an antigen are known in the art. The antibody can be immobilized on a solid support such as a stick, plate, bead, microbead or array.

Expression levels of one or more of the genes described herein can be also be determined indirectly by determining the mRNA expression for the one or more genes in a tissue sample. RNA expression methods include but are not limited to extraction of cellular mRNA and Northern blotting using labeled probes that hybridize to transcripts encoding all or part of the gene, amplification of mRNA using gene-specific primers, polymerase chain reaction (PCR), and reverse transcriptase-polymerase chain reaction (RT-PCR), followed by quantitative detection of the gene product by a variety of methods; extraction of RNA from cells, followed by labeling, and then used to probe cDNA or oligonucleotides encoding the gene, in situ hybridization; and detection of a reporter gene.

Methods to measure protein expression levels include but are not limited to Western blot, immunoblot, ELISA, radioimmunoassay, immunoprecipitation, surface plasmon resonance, chemiluminescence, fluorescent polarization, phosphorescence, immunohistochemical analysis, microcytometry, microarray, microscopy, fluorescence activated cell sorting (FACS), and flow cytometry. The method can also include specific protein property-based assays based including but not limited to enzymatic activity or interaction with other protein partners. Binding assays can also be used, and are well known in the art. For instance, a BIAcore machine can be used to determine the binding constant of a complex between two proteins. Other suitable assays for determining or detecting the binding of one protein to another include, immunoassays, such as ELISA and radioimmunoassays. Determining binding by monitoring the change in the spectroscopic can be used or optical properties of the proteins can be determined via fluorescence, UV absorption, circular dichroism, or nuclear magnetic resonance (NMR). Alternatively, immunoassays using specific antibody can be used to detect the expression on of a particular protein on a tumor cell.

In some aspects, the gene expression level can be determined by quantitative (q) PCR, RNA sequencing (RNA-seq), next-generation sequencing (NGS), or a combination thereof. In some aspects, the protein expression level can be determined by immunohistochemical, ELISA, or a combination thereof.

Reference expression level. As used herein, the term “reference,” “reference expression,” “reference sample,” “reference value,” “control,” “control sample” and the like, when used in the context of a sample or expression level of one or more genes or proteins refers to a reference standard wherein the reference is expressed at a constant level among different (i.e., not the same tissue, but multiple tissues) tissues, and is unaffected by the experimental conditions, and is indicative of the level in a sample of a predetermined disease status (e.g., not suffering from glioblastoma or breast cancer). The reference value can be a predetermined standard value or a range of predetermined standard values, representing no illness, or a predetermined type or severity of illness.

Reference gene expression can be the level of the one or more genes described herein in a reference sample from a subject, or a pool of subjects, not suffering from glioblastoma or from a predetermined severity or type of brain cancer or glioblastoma. In some aspects, the reference value is the level of one or more genes disclosed herein in the tissue of a subject, or subjects, wherein the subject or subjects is not suffering from brain cancer or glioblastoma. In some aspects, the expression level of each gene can be normalized to the expression level of one or more reference genes. In some aspects, the gene expression of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 can be normalized to the expression level of one or more reference genes.

Reference gene expression can be the level of the one or more genes described herein in a reference sample from a subject, or a pool of subjects, not suffering from breast cancer or from a predetermined severity or type of breast cancer. In some aspects, the reference value is the level of one or more genes disclosed herein in the tissue of a subject, or subjects, wherein the subject or subjects is not suffering from breast cancer. In some aspects, the expression level of each gene can be normalized to the expression level of one or more reference genes. In some aspects, the gene expression of PGK1, NQO1, HMOX1, VEGFA, ADM, HPCAL1, PLK3, FOSL1, and PLAU can be normalized to the expression level of one or more reference genes.

In some aspects, the methods can include a step comprising correcting for (normalize away) differences in the amount of RNA assayed and/or variability in the quality of the RNA used. Therefore, assays and methods of the invention may measure and incorporate the expression of certain normalizing genes, including well known housekeeping genes. Nonlimiting examples of normalizing genes include ABCF1, ACTB, ALAS1, CLTC, G6PD, GAPDH, GUSB, HPRT1, LDHA, PGK1, POLR1B, POLR2A, RPL19, RPLP0, SDHA, TBP, RNA18SN5 (the ribosomal 18S subunit) and/or TUBB. In some aspects, a combination of two or more normalizing genes may be used. In some aspects, normalization can be based on the mean or median signal (Ct) of the assayed genes or a large subset thereof (global normalization approach).

In some aspects, sample-specific normalization factors can be used to normalize raw mRNA counts in order to account for slight differences in assay efficiency such as hybridization, purification, and binding. In some aspects, normalization for sample RNA quantity and quality differences are applied to spike-normalized values using sample-specific normalization factors calculated from the geometric mean of the counts from reporters targeting the reference genes, including but not limited to any one of or all of the following reference genes: ABCF1, ACTB, ALAS1, CLTC, G6PD, GAPDH, GUSB, HPRT1, LDHA, PGK1, POLR1B, POLR2A, RPL19, RPLP0, SDHA, TBP, and TUBB. The resulting normalized counts may be used in downstream analyses.

Comparing the expression level of one or more genes disclosed herein. By comparing the expression level for one or more of, for example, DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 of step b) with the reference expression level (or predetermined reference level) for, for example, DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 of step c), it is possible to identify a subject with an increased risk of short survival and/or recurrence of glioblastoma.

By comparing the expression level for one or more of, for example, PGK1, NQO1, HMOX1, VEGFA, ADM, HPCAL1, PLK3, FOSL1, and PLAU of step b) with the reference expression level (or predetermined reference level) for, for example, PGK1, NQO1, HMOX1, VEGFA, ADM, HPCAL1, PLK3, FOSL1, and PLAU of step c), it is possible to identify a subject with an increased risk of short survival and/or recurrence of breast cancer.

Determining the expression level of one or more genes disclosed herein can include determining whether the gene is upregulated or increased as compared to a control or reference sample, downregulated or decreased compared to a control or reference sample, or unchanged compared to a control or reference sample. As used herein, the terms, “upregulated” and “increased expression level” or “increased level of expression” refers to a sequence corresponding to one or more genes disclosed herein that is expressed wherein the measure of the quantity of the sequence exhibits an increased level of expression when compared to a reference sample or “normal” control. For example, the terms, “upregulated” and “increased expression level” or “increased level of expression” refers to a sequence corresponding to one or more genes disclosed herein that is expressed wherein the measure of the quantity of the sequence exhibits an increased level of expression of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 protein(s) and/or mRNA when compared to the expression of the same mRNA(s) from a reference sample or “normal” control. An “increased expression level” refers to an increase in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. As used herein, the terms “downregulated,” “decreased level of expression,” or “decreased expression level” refers to a sequence corresponding to one or more genes disclosed herein that is expressed wherein the measure of the quantity of the sequence exhibits a decreased level of expression when compared to a reference sample or “normal” control For example, the terms “downregulated,” “decreased level of expression,” or “decreased expression level” refers to a sequence corresponding to one or more genes disclosed herein that is expressed wherein the measure of the quantity of the sequence exhibits a decreased level of expression of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 protein(s) and/or mRNA when compared to the expression of the same mRNA(s) from a reference sample or “normal” control. A “decreased level of expression” refers to a decrease in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more.

Determining an increased risk of short survival and/or recurrence to glioblastoma. As described herein, samples from a subject can be compared with reference samples to determine the expression ratio to determine whether a subject has an increased risk of short survival and/or recurrence of glioblastoma. The reference samples can be from subjects having “normal” levels of one or more of the following genes, DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1. Suitable statistical and other analysis can be carried out to confirm a change (e.g., an increase or a higher level of expression) in one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 when compared with a reference sample, wherein a ratio of the sample expression level of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 to the reference expression level of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 indicates higher expression level of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample. In some aspects, the ratio of the sample expression level of two or more, three or more, four or more, five or more, or six or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 to the reference expression level of two or more, three or more, four or more, five or more, or six or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 indicates higher expression level of two or more, three or more, four or more, five or more, or six or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample, indicating that the subject has an increased risk of short survival and/or recurrence of glioblastoma. In some aspects, the one or more genes can be PGK1, NQO1, HMOX1, VEGFA, ADM, HPCAL1, PLK3, FOSL1, and PLAU.

A higher or increased expression level of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 when compared to the reference expression level of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 can indicate an increased risk of short survival and/or recurrence of glioblastoma. Signature pattern(s) of increased (higher) or decreased (lower) sample expression levels of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1when compared to the reference expression levels of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1can be observed and indicate an increased risk of short survival and/or recurrence of glioblastoma in a subject. In some aspects, the one or more genes can be PGK1, NQO1, HMOX1, VEGFA, ADM, HPCAL1, PLK3, FOSL1, and PLAU.

The gene expression level of one or more genes described herein can be a measure of one or more genes, for example, per unit weight or volume. In some aspects, the expression level can be a ratio (e.g., the amount of one or more genes in a sample relative to the amount of the one or more markers of a reference value).

In some aspects, samples from a subject can be compared with reference samples to determine the percent change to identify a subject with an increased risk of short survival and/or recurrence of glioblastoma. In other words, the expression level can be expressed as a percent. For example, the percent change in the expression levels of one or more genes, wherein the expression level of one (or two, three, four, five or six) or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 is increased (or is higher) by 10%, 15%, 20%, 25%, 30%, 35%, 40%, 45%, 50%, 55%, 60%, 65%, 70%, 75%, 80%, 85%, 90%, 95%, or 100% when compared to the reference expression level of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1, indicating an risk of short survival and/or recurrence of glioblastoma. Alternatively, the percent change in the expression levels of one or more genes can be decreased (or lower) by 10%, 15%, 20%, 25%, 30%, 35%, 40%, 45%, 50%, 55%, 60%, 65%, 70%, 75%, 80%, 85%, 90%, 95%, or 100% when compared to a reference expression level. In some aspects, the one or more genes can be PGK1, NQO1, HMOX1, VEGFA, ADM, HPCAL1, PLK3, FOSL1, and PLAU.

In some aspects, an increase or decrease or some combination thereof in the expression level of genes or proteins other than those disclosed herein can indicate an risk of short survival and/or recurrence of glioblastoma or a diagnosis of glioblastoma in a subject. In some aspects, a signature pattern of increased or decreased expression levels of one or more of the genes or proteins disclosed herein is indicative.

In some aspects, the methods disclosed herein can further include a method of preventing of recurrence of glioblastoma morbidity and/or mortality. For example, the method comprises providing to a subject, further testing (which can include testing for cancer), such as, for example, a computed tomography, magnetic resonance imaging, and/or a routine physical examination, wherein an increased risk of short survival and/or recurrence of glioblastoma has been diagnosed. The method can further include the administration of therapy to prevent glioblastoma from developing or spreading, thereby reducing glioblastoma morbidity and/or mortality.

The methods described herein can further comprise the step of assaying the brain tissue sample from the subject to detect the presence of other molecular features of brain cancer or glioblastoma. In some aspects, the method can further comprise the step of assaying the brain tissue sample from the subject to determine the Ki67 gene or Ki67 protein level or expression level. In some aspects, wherein a high level of the Ki67 gene or protein is detected in the sample, the ratio (or percent change) of the sample expression level of at least one of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 to reference expression of the same gene is two-fold higher (or more) indicating increased risk of short survival and/or recurrence of glioblastoma in the subject.

Determining an increased risk of short survival and/or recurrence to breast cancer. As described herein, samples from a subject can be compared with reference samples to determine the expression ratio to determine whether a subject has an increased risk of short survival and/or recurrence of breast cancer. The reference samples can be from subjects having “normal” levels of one or more of the following genes, PGK1, NQO1, HMOX1, VEGFA, ADM, HPCAL1, PLK3, FOSL1, and PLAU. Suitable statistical and other analysis can be carried out to confirm a change (e.g., an increase or a higher level of expression) in one or more of PGK1, NQO1, HMOX1, VEGFA, ADM, HPCAL1, PLK3, FOSL1, and PLAU when compared with a reference sample, wherein a ratio of the sample expression level of one or more of PGK1, NQO1, HMOX1, VEGFA, ADM, HPCAL1, PLK3, FOSL1, and PLAU to the reference expression level of one or more of PGK1, NQO1, HMOX1, VEGFA, ADM, HPCAL1, PLK3, FOSL1, and PLAU indicates higher expression level of one or more of PGK1, NQO1, HMOX1, VEGFA, ADM, HPCAL1, PLK3, FOSL1, and PLAU in the sample. In some aspects, the ratio of the sample expression level of two or more, three or more, four or more, five or more, or six or more of In some aspects, the one or more genes can be PGK1, NQO1, HMOX1, VEGFA, ADM, HPCAL1, PLK3, FOSL1, and PLAU to the reference expression level of two or more, three or more, four or more, five or more, or six or more of PGK1, NQO1, HMOX1, VEGFA, ADM, HPCAL1, PLK3, FOSL1, and PLA U indicates higher expression level of two or more, three or more, four or more, five or more, or six or more of PGK1, NQO1, HMOX1, VEGFA, ADM,HPCAL1, PLK3, FOSL1, and PLAU in the sample, indicating that the subject has an increased risk of short survival and/or recurrence of breast cancer.

A higher or increased expression level of one or more of PGK1, NQO1, HMOX1, VEGFA, ADM, HPCAL1, PLK3, FOSL1, and PLAU when compared to the reference expression level of PGK1, NQO1, HMOX1, VEGFA, ADM, HPCAL1, PLK3, FOSL1, and PLAU can indicate an increased risk of short survival and/or recurrence of breast cancer. Signature pattern(s) of increased (higher) or decreased (lower) sample expression levels of one or more of PGK1, NQO1, HMOX1, VEGFA, ADM, HPCAL1, PLK3, FOSL1, and PLAU when compared to the reference expression levels of one or more of PGK1, NQO1, HMOX1, VEGFA, ADM, HPCAL1, PLK3, FOSL1, and PLAU can be observed and indicate an increased risk of short survival and/or recurrence of breast cancer in a subject.

The gene expression level of one or more genes described herein can be a measure of one or more genes, for example, per unit weight or volume. In some aspects, the expression level can be a ratio (e.g., the amount of one or more genes in a sample relative to the amount of the one or more markers of a reference value).

In some aspects, samples from a subject can be compared with reference samples to determine the percent change to identify a subject with an increased risk of short survival and/or recurrence of breast cancer. In other words, the expression level can be expressed as a percent. For example, the percent change in the expression levels of one or more genes, wherein the expression level of one (or two, three, four, five or six) or more of PGK1, NQO1, HMOX1, VEGFA, ADM, HPCAL1, PLK3, FOSL1, and PLAU is increased (or is higher) by 10%, 15%, 20%, 25%, 30%, 35%, 40%, 45%, 50%, 55%, 60%, 65%, 70%, 75%, 80%, 85%, 90%, 95%, or 100% when compared to the reference expression level of PGK1, NQO1, HMOX1, VEGFA, ADM, HPCAL1, PLK3, FOSL1, and PLAU, indicating an risk of short survival and/or recurrence of breast cancer. Alternatively, the percent change in the expression levels of one or more genes can be decreased (or lower) by 10%, 15%, 20%, 25%, 30%, 35%, 40%, 45%, 50%, 55%, 60%, 65%, 70%, 75%, 80%, 85%, 90%, 95%, or 100% when compared to a reference expression level.

In some aspects, an increase or decrease or some combination thereof in the expression level of genes or proteins other than those disclosed herein can indicate a risk of short survival and/or recurrence of breast cancer or a diagnosis of breast cancer in a subject. In some aspects, a signature pattern of increased or decreased expression levels of one or more of the genes or proteins disclosed herein is indicative.

In some aspects, the methods disclosed herein can further include a method of preventing of recurrence of breast cancer morbidity and/or mortality. For example, the method comprises providing to a subject, further testing (which can include testing for cancer), such as, for example, a mammogram, an ultrasound, a computed tomography, magnetic resonance imaging, and/or a routine physical examination, wherein an increased risk of short survival and/or recurrence of breast cancer has been diagnosed. The method can further include the administration of therapy to prevent breast cancer from developing or spreading, thereby reducing breast cancer morbidity and/or mortality.

The methods described herein can further comprise the step of assaying the breast tissue sample from the subject to detect the presence of other molecular features of breast cancer. In some aspects, the method can further comprise the step of assaying the breast tissue sample from the subject to determine the Ki67 gene or Ki67 protein level or expression level. In some aspects, wherein a high level of the Ki67 gene or protein is detected in the sample, the ratio (or percent change) of the sample expression level of at least one of PGK1, NQO1, HMOX1, VEGFA, ADM, HPCAL1, PLK3, FOSL1, and PLAU to reference expression of the same gene is two-fold higher (or more) indicating increased risk of short survival and/or recurrence of breast cancer in the subject.

### Methods of Screening

Disclosed herein are methods of screening for a therapeutic agent. In some aspects, the methods can comprise placing a cell or a population of cells from a brain tissue sample in an integrative microfluidic apparatus. In some aspects, the methods can comprise placing a cell or a population of cells from a breast tissue sample in an integrative microfluidic apparatus. In some aspects, the integrative microfluidic apparatus can comprise a migratory channel. In some aspects, the integrative microfluidic apparatus can comprise two or more (or a series) migratory channels. In some aspects, the migratory channel can have an inlet end and two or more outlet ends. In some aspects, each of the migratory channels can have an inlet end and two or more outlet ends. The two or more outlet ends are the result of a bifurcation (or trifurcation) point in the migratory channel and a point distal from the inlet end of the migratory channel. An integrative microfluidic apparatus useful in the claimed methods is described in PCT/US2016/064725. PCT/US2016/064725 is hereby incorporated herein by reference in its entirety. In some aspects, the methods can comprise determining whether the cell or the population of cells migrates through one of the migratory channels of the apparatus and to the corresponding bifurcation point of the migratory channel in the presence and absence of the therapeutic agent. In some aspects, the methods can comprise determining that the therapeutic agent is an inhibitor of cancer cell migration when the cell or population of cells does not migrate through the migratory channel of the apparatus and to the bifurcation point of the channel in the presence of the therapeutic agent. In some aspects, the therapeutic agent can be placed in a media that can fill the integrative microfluidic apparatus. The cell or population of cells can then be immersed in the media. In some aspects, the cell or population of cells can be seeded or introduced to the integrative microfluidic apparatus via the second channel. In some aspects, the integrative microfluidic apparatus can be used to create a concentration gradient across the two or more migratory channels, such that the further the cells or the population of cells migrate into the one of the two or more migratory channels, the more or less drug concentration the cells or the population of cells contacted (depending on which direction the gradient was set). In some aspects, the cell or population of cells can be brain cancer cells. In some aspects, the brain cancer cells can be glioblastoma cells. In some aspects, the cell or population of cells are isolated from resected brain tumor tissue. In some aspects, the resected brain tumor tissue is from a subject diagnosed with or suspected of having glioblastoma. In some aspects, the brain tissue sample can be from a subject diagnosed with glioblastoma. In some aspects, the cell or population of cells can be breast cancer cells. In some aspects, the brain cancer cells can be any type of breast cancer cells. In some aspects, the cell or population of cells are isolated from resected breast tumor tissue. In some aspects, the resected breast tumor tissue is from a subject diagnosed with or suspected of having breast cancer. In some aspects, the breast tissue sample can be from a subject diagnosed with breast cancer. In some aspects, the cell or population of cells can be drug-naive. In some aspects, the cell or population of cells have not been previously exposed to any therapeutic agent. In some aspects, the cell or population of cells have not been previously exposed to any therapeutic agent that can be used to treat brain cancer or glioblastoma. In some aspects, the cell or population of cells have not been subjected to radiation therapy or chemotherapy (e.g., temozolomide). In some aspects, the cell or population of cells have not been previously exposed to any therapeutic agent that can be used to treat breast cancer. In some aspects, the cell or population of cells have not been subjected to radiation therapy or chemotherapy (e.g., temozolomide).

Disclosed herein are methods of screening for therapeutic agents that inhibit cancer cell migration. In some aspects, the methods can comprise: a) placing a cell or a population of cells from a tissue sample in an integrative microfluidic apparatus, wherein the integrative microfluidic apparatus comprises a migratory channel and a bifurcation point in the channel; b) collecting one or more cells from the cell or the population of cells that migrate through the migratory channel of the apparatus and to the bifurcation point of the channel; c) optionally determining an increased gene expression level of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the one or more cells from step b); d) contacting one or more cells from step b) with a therapeutic agent; e) placing the one or more cells from step d) into the integrative microfluidic apparatus of step a); f) determining whether the one or more cells from step e) migrates through the migratory channel of the apparatus and to the bifurcation point of the channel in the presence of the therapeutic agent; wherein the therapeutic agent is an inhibitor of cancer cell migration when one or more cells from step f) do not migrate through the migratory channel of the apparatus and to the bifurcation point of the channel in the presence of the therapeutic agent.

In some aspects, step d) can be performed inside the integrative microfluidic apparatus of step e). In some aspects, the cells of step d) can have an increased gene expression levels of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1.

Disclosed herein are methods of screening for therapeutic agents that inhibit cancer cell migration. In some aspects, the methods can comprise: a) obtaining or having obtained a sample from a subject, wherein the sample comprises a population of cells; b) determining an increased gene expression levels of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in one or more cells of the population of cells from step a); c) contacting the one or more cells from step a) with a therapeutic agent; d) placing the one or more cells from step c) into the integrative microfluidic apparatus of step a); e) determining whether the one or more cells from step d) migrates through the migratory channel of the apparatus and to the bifurcation point of the channel in the presence of the therapeutic agent; wherein the therapeutic agent is an inhibitor of cancer cell migration when one or more cells from step e) do not migrate through the migratory channel of the apparatus and to the bifurcation point of the channel in the presence of the therapeutic agent. In some aspects, step c) can be performed inside the integrative microfluidic apparatus of step d).

In some aspects, the brain tissue sample can comprise a higher level of gene expression of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 compared to a predetermined reference level of gene expression of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1.

In some aspects, the breast tissue sample can comprise a higher level of gene expression of one or more of PGK1, NQO1, HMOX1, VEGFA, ADM, HPCAL1, PLK3, FOSL1, and PLAU compared to a predetermined reference level of gene expression of one or more of PGK1, NQO1, HMOX1, VEGFA, ADM, HPCAL1, PLK3, FOSL1, and PLAU.

In some aspects, the methods can further comprise determining that the therapeutic agent is an inhibitor of cancer cell proliferation. In some aspects, cancer cell proliferation can be determined by quantifying Ki67 expression in the cell or population of cells.

### Diagnostic Device

Disclosed herein, are diagnostic devices for diagnosing or assessing the risk of recurrence of glioblastoma in a subject (e.g., human). Also disclosed herein, are diagnostic devices for diagnosing or assessing the risk of recurrence of breast in a subject (e.g., human). In some aspects, a sample of tissue can be obtained from the subject and the level or expression level in the sample can be compared with a reference value. In some aspects, the tissue can be brain tissue. In some aspects, the tissue can be breast tissue.

The diagnostic device can include one or more biomarkers. In some aspects, the diagnostic device can include 2, 3, 4, 5, 6, etc. or more biomarkers. Biomarkers can bind to or hybridize with one or more genes disclosed herein, RNA products or peptides. As used herein, the terms “marker” or “biomarker” refers to detectable or measurable substance (e.g., gene, gene product, protein, etc.) in a sample that can indicate a biological state, disease, condition, predict a clinical outcome, etc. In some aspects, biomarkers can be one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 or one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 or FTH1 or a fragment thereof, or an antibody or fragment thereof which binds one or more of the biomarkers. The diagnostic device can be incorporated into a kit for diagnosing or assessing the risk of recurrence of glioblastoma in a subject. In some aspects, biomarkers can be one or more of PGK1, NQO1, HMOX1, VEGFA, ADM, HPCAL1, PLK3, FOSL1, and one or more of PLAU or PGK1, NQO1, HMOX1, VEGFA, ADM, HPCAL1, PLK3, FOSL1, and PLAU or a fragment thereof, or an antibody or fragment thereof which binds one or more of the biomarkers. The diagnostic device can be incorporated into a kit for diagnosing or assessing the risk of recurrence of breast cancer in a subject.

### Protein Array

Disclosed herein are polypeptide or protein arrays. In some aspects, the protein arrays can comprise probes including antibodies, aptamers, and other cognate binding ligands specific to a component of the gene panels disclosed herein. Protein arrays and methods of constructing the protein arrays are well known to one of ordinary skill in the art.

One type of protein array that can be suitable uses an immobilized “capture antibody.’ The polypeptides are bound to a solid substrate (e.g., glass) with a treated surface (e.g., aminosilane) or through a biotin-streptavidin conjugation. The arrays are then incubated with a solution containing probe that can bind to the capture antibodies in a manner dependent upon time, buffer components, and recognition specificity. The probes can then be visualized directly if they have been previously labeled, or can be bound to a secondary labeled reagent (e.g., another antibody). The amount of probe bound to the capture antibody that is visualized can depend upon the labeling method utilized; generally, a CCD imager or laser scanner that uses filter sets that are appropriate to excite and detect the emissions of the label can be used. The imager converts the amount of detected photons into an electronic signal (often an 8-bit or 16-bit scale) that can be analyzed using commercially available software packages.

The substrate of the array can be organic or inorganic, biological or non-biological or any combination of these materials. The substrate can be transparent or translucent. Examples of materials suitable for use as a substrate in the array include silicon, silica, quartz, glass, controlled pore glass, carbon, alumina, titanium dioxide, germanium, silicon nitride, zeolites, and gallium arsenide; and metals including gold, platinum, aluminum, copper, titanium, and their alloys. Ceramics and polymers can also be used as substrates. Suitable polymers include, but are not limited to polystyrene; poly(tetra)fluorethylene; (poly)vinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PM I); polyalkenesulfone (PAS); polyhydroxyethylmethacrylate; polydimethylsiloxane; polyacrylamide; polyimide; co-block-polymers; and Eupergit®. Photoresists, polymerized Langmuir-Blodgett films, and LIGA structures can also serve as substrates.

The array can further comprise a coating that can be formed on the substrate or applied to the substrate. The substrate can be modified with a coating by using thin-film technology based on either physical vapor deposition (PVD) or plasma-enhanced chemical vapor deposition (PECVD). Alternatively, plasma exposure can be used to directly activate the substrate. For instance, plasma etch procedures can be used to oxidize a polymeric surface (i.e., polystyrene or polyethylene to expose polar functionalities such as hydroxyls, carboxylic acids, aldehydes and the like).

The coating can comprise a metal film. Examples of metal films include aluminum, chromium, titanium, nickel stainless steel zinc, lead, iron, magnesium, manganese, cadmium, tungsten, cobalt, and alloys or oxides thereof. In some aspects, the metal film can be a noble metal film. Examples of noble metals that can be used for a coating include, but are not limited to, gold, platinum, silver, copper, and palladium. In some aspects, the coating comprises gold or a gold alloy. Electron-beam evaporation can be used to provide a thin coating of gold on the surface. In some aspects, the metal film can from about 50 nm to about 500 nm in thickness.

Alternatively, the coating can be silicon, silicon oxide, silicon nitride, silicon hydride, indium tin oxide, magnesium oxide, alumina, glass, hydroxylated surfaces, and a polymer.

The arrays described herein can comprise a collection of addressable elements. Such elements can be spatially addressable, such as arrays contained within microtiter plates or printed on planar surfaces wherein each element can be present at distinct X and Y coordinates. Alternatively, elements can be addressable based on tags, beads, nanoparticles, or physical properties. The microarrays can be prepared according to the methods known to one of ordinary skill in the art. The term “arrays” as used herein can refer to any biologic assay with multiple addressable elements. In some aspects, the addressable elements can be polypeptides (e.g., antibodies or fragments thereof) or nucleic acid probes. As used herein, “elements” refer to any probe (polypeptide or nucleic acid based) that can be bound by an organ-specific polypeptide, polypeptide fragment or transcript encoding such polypeptides, as related or associated with any of the gene or proteins disclosed herein. Molecules can be, but are not limited to, proteins, polypeptides, peptides, RNA, DNA, lipids, glycosylated molecules, carbohydrates, polypeptides with phosphorylation modifications, and polypeptides with citrulline modifications, aptamers, oxidated molecules, and other molecules.

For the elements described herein, “addressability” refers to the location, position, tags, cleavable tags or markers, identifiers, spectral properties, electrophoretic properties, or other physical properties that enable identification of the element. An example of addressability, also known as coding, is spatial addressability, where the position of the molecule is fixed, and that position is correlated with the identity. This type of spatial array can generally be synthesized or spotted onto a planar substrate, producing, for example, microarrays, where a large number of different molecules are densely laid out in a small area (e.g. comprising at least about 400 different sequences per cm², and can be 1000 sequences per cm² or as many as 5000 sequences per cm², or more). Less dense arrays (e.g., ELISA or RIA plates) where wells in a plate each contain a distinct probe can comprise from about 96 sequences per plate, up to about 100 sequences per cm², up to the density of a microarray. Other spatial arrays utilize fiber optics, where distinct probes can be bound to fibers, which can be formed into a bundle for binding and analysis. Methods for the manufacture and use of spatial arrays of polypeptides are known in the art.

An alternative to this type of spatial coding array is the use of molecular “tags,” where the target probes can be attached to a detectable label, or tag, which can provide coded information about the sequence of the probe. These tags can be cleaved from the element, and subsequently detected to identify the element. In some aspects, a set of probes can be synthesized or attached to a set of coded beads, wherein each bead can be linked to a distinct probe, and wherein the beads can be coded in a manner that allows identification of the attached probe. In this type of “tag array,” flow cytometry can be used for detection of binding. For example, microspheres having fluorescence coding and can identify a particular microsphere. The probe can be covalently bound to a “color coded” object. A labeled target polypeptide can be detected by flow cytometry, and the coding on the microsphere can be used to identify the bound probe (e.g., immunoglobulin, antigen binding fragments of immunoglobulins, or ligands).

In some aspects, the array can be an immunoglobulin (e.g., antibody or antigen-binding fragment thereof) array. As used herein, an “immunoglobulin array” refers to a spatially separated set of discrete molecular entities capable of binding to target polypeptides arranged in a manner that allows identification of the polypeptides contained within the sample. In some aspects, the array can comprise one or more of proteins, polypeptides, peptides, RNA, DNA, lipid, glycosylated molecules, polypeptides with phosphorylation modifications, and polypeptides with citrulline modifications, aptamers, and other molecules.

### Kits

In some aspects, kits are provided for measuring the RNA (e.g., a RNA product) of one or more biomarkers or genes (or proteins) disclosed herein. The kits can comprise materials and reagents that can be used for measuring the expression of the RNA of one or more biomarkers. Examples of suitable kits include RT-PCR or microarray. These kits can include the reagents needed to carry out the measurements of the RNA expression levels. Alternatively, the kits can further comprise additional materials and reagents. For example, the kits can comprise materials and reagents required to measure RNA expression levels of any number of genes up to 1, 2, 3, 4, 5, 6, 7, 8, 9, 10, or more genes that are not biomarkers disclosed herein.

### Gene Expression Panel

Disclosed herein are gene expression panels and arrays for assessing risk of recurrence of glioblastoma in a subject (e.g., human) consisting of primers or probes capable of detecting one or more genes disclosed herein. The disclosed gene expression panels or arrays can comprise any of the genes disclosed herein. For example, the gene expression panel or array can be used to detect one or more of DUSP5, PLK3, PPP1R15A, FOSL1, CDKN1A, KLF6, VDR, ARL4C, ADM, PLAU, VEGFA, NOO1, HMOX1, PGK1, LITAF, HPCAL1 and FTH1. In some aspects, the gene expression panels or arrays can comprise DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1. In some aspects, the gene expression pane or array can comprise one or more primers or probes capable of detecting one or more of DUSP5, PLK3, PPP1R15A, FOSL1, CDKN1A, KLF6, VDR, ARL4C, ADM, PLAU, VEGFA, NOO1, HMOX1, PGK1, LITAF, HPCAL1 and FTH1.

In some aspects, the sample can be brain tissue. The brain tissue can be from a resected brain tumor. In some aspects, the brain tumor can be glioblastoma.

Disclosed herein are gene expression panels and arrays for assessing risk of recurrence of breast cancer in a subject (e.g., human) consisting of primers or probes capable of detecting one or more genes disclosed herein. The disclosed gene expression panels or arrays can comprise any of the genes disclosed herein. For example, the gene expression panel or array can be used to detect one or more of PGK1, NOO1, HMOX1, VEGFA, ADM, HPCAL1, PLK3, FOSL1, and PLAU. In some aspects, the gene expression panels or arrays can comprise PGK1, NQO1, HMOX1, VEGFA, ADM, HPCAL1, PLK3, FOSL1, and PLAU. In some aspects, the gene expression pane or array can comprise one or more primers or probes capable of detecting one or more of PGK1, NQO1, HMOX1, VEGFA, ADM, HPCAL1, PLK3, FOSL1, and PLAU.

In some aspects, the sample can be breast tissue. The brain tissue can be from a resected breast tumor. In some aspects, the breast tumor can be ductal carcinoma in situ, invasive ductal carcinoma, inflammatory breast cancer or metastatic breast cancer.

The gene expression panels or arrays disclosed herein can consist of primers or probes capable of detecting or amplifying any number of the genes disclosed herein. The gene expression panels or arrays disclosed herein can further comprise primers or probes capable of detecting or amplifying any number of genes not disclosed herein. For example, the primers or probes can detect or amplify between 1 and 5, 5 and 10, 10 and 100, or more, or any variation in between.

Disclosed herein are methods for detecting or amplifying one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 using the gene expression panels or arrays disclosed herein. In some aspects, the methods can be used to identify a subject at risk for recurrence of glioblatoma. In some aspects, the methods can comprise contacting a sample with the gene expression panel or array disclosed herein.

Disclosed herein are methods for detecting or amplifying one or more of PGK1, NQO1,HMOX1, VEGFA, ADM, HPCAL1, PLK3, FOSL1, and PLAU using the gene expression panels or arrays disclosed herein. In some aspects, the methods can be used to identify a subject at risk for recurrence of breast cancer. In some aspects, the methods can comprise contacting a sample with the gene expression panel or array disclosed herein.

The gene expression panels or arrays disclosed herein can be used as a standalone method for assessing risk of recurrence of glioblastoma or breast cancer in a subject or in combination with one or more other gene expression panels or arrays not disclosed herein. They can be used along with one or more diagnostic test. In some aspects, the gene expression panels or arrays can further comprise a second diagnostic test. The gene expression panels or arrays disclosed herein can also be used in methods to generate a specific profile. The profile can be provided in the form of a heatmap or boxplot.

The profile of the gene expression levels can be used to compute a statistically significant value based on differential expression of the one or more genes disclosed herein, wherein the computed value correlates to a diagnosis for an increased risk of short survival and/or recurrence of glioblastoma. The variance in the obtained profile of expression levels of the selected genes or gene expression products can be either upregulated or downregulated in subjects with an increased risk of recurrence of glioblastoma compared to a reference subject or control. The Examples section provides additional detail. For instance, when the expression level of DUSP5, PLK3, PPP1R15A, FOSL1, CDKN1A, KLF6, VDR, ARL4C, ADM, PLAU, VEGFA, NOO1, HMOX1, PGK1, LITAF, HPCAL1 and FTH1are upregulated, an increased risk of recurrence of glioblastoma is indicated. As described herein, one of ordinary skill in the art can use a combination of any of genes disclosed herein to form a profile that can then be used to assess risk of recurrence of glioblastoma, or to determine (and diagnose) whether a subject has glioblastoma or a short survival.

Disclosed herein are methods of diagnosing glioblastoma using the gene expression panel or array described herein. In some aspects, the method further comprises quantifying cell invasion using a microfluidic assay. In some aspects, the method further comprises determining the invasiveness of a cell or a population of cells from the brain tissue sample.

In some aspects, the gene expression panel or array disclosed herein can be used to identify or determine or assess the risk of short survival and/or recurrence of glioblastoma in a subject. In some aspects, the expression level for one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1in a sample can be compared to a predetermined reference expression level for one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL 1 and FTH1. In some aspects, the gene expression panel or array disclosed herein can be used to identify or determine or assess the risk of short survival and/or recurrence of glioblastoma in a subject, wherein a ratio (or percent change) of the sample expression level of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1to the reference expression level of one or more of DUSP5, PLK3, PPP1R15A, FOSL1, CDKNI1A, KLF6, VDR, ARL4C, ADM, PLAU, VEGFA, NOO1, HMOX1, PGK1, LITAF, HPCAL1 and FTH1indicates higher expression level of one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample. In some aspects, the ratio (or percent change) of the sample expression level of two or more, three or more, four or more, five or more, or six or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1to the reference expression level of two or more, three or more, four or more, five or more, or six or more of DUSP5, PLK3, PPP1R15A, FOSL1, CDKN1A, KLF6, VDR, ARL4C, ADM, PLAU, VEGFA, NOO1, HMOX1, PGK1, LITAF, HPCAL1 and FTH1 indicates higher expression level of two or more, three or more, four or more, five or more, or six or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1 in the sample, indicating that the subject has an increased risk of short survival and/or recurrence of glioblastoma. Suitable statistical and other analysis can be carried out to confirm a change (e.g., an increase or a higher level of expression) in one or more of DUSP5, PLK3, PPP1R15A, FOSL1, CDKN1A, KLF6, VDR, ARL4C, ADM, PLAU, VEGFA, NQO1, HMOX1, PGK1, LITAF, HPCAL1 and FTH1when compared with a reference sample.

The profile of the gene expression levels can be used to compute a statistically significant value based on differential expression of the one or more genes disclosed herein, wherein the computed value correlates to a diagnosis for an increased risk of short survival and/or recurrence of breast cancer. The variance in the obtained profile of expression levels of the selected genes or gene expression products can be either upregulated or downregulated in subjects with an increased risk of recurrence of breast cancer compared to a reference subject or control. The Examples section provides additional detail. For instance, when the expression level of PGK1, NQO1, HMOX1, VEGFA, ADM, HPCAL1, PLK3, FOSL1, and PLAU are upregulated, an increased risk of recurrence of breast cancer is indicated. As described herein, one of ordinary skill in the art can use a combination of any of genes disclosed herein to form a profile that can then be used to assess risk of recurrence of breast cancer, or to determine (and diagnose) whether a subject has breast cancer or a short survival.

Disclosed herein are methods of diagnosing breast cancer using the gene expression panel or array described herein. In some aspects, the method further comprises quantifying cell invasion using a microfluidic assay. In some aspects, the method further comprises determining the invasiveness of a cell or a population of cells from the breast tissue sample.

In some aspects, the gene expression panel or array disclosed herein can be used to identify or determine or assess the risk of short survival and/or recurrence of breast cancer in a subject. In some aspects, the expression level for one or more of PGK1, NQO1,HMOX1, VEGFA, ADM, HPCAL1, PLK3, FOSL1, and PLAU in a sample can be compared to a predetermined reference expression level for one or more of PGK1, NQO1,HMOX1, VEGFA, ADM, HPCAL1, PLK3, FOSL1, and PLAUIn some aspects, the gene expression panel or array disclosed herein can be used to identify or determine or assess the risk of short survival and/or recurrence of breast cancer in a subject, wherein a ratio (or percent change) of the sample expression level of one or more of PGK1, NQO1, HMOX1, VEGFA, ADM, HPCAL1, PLK3, FOSL1, and PLAUto the reference expression level of one or more of PGK1, NQOI, HMOX1, VEGFA, ADM, HPCAL 1, PLK3, FOSL1, and PLA U indicates higher expression level of one or more of PGK1, NQO1,HMOX1, VEGFA, ADM, HPCAL1, PLK3, FOSL1, and PLAU in the sample. In some aspects, the ratio (or percent change) of the sample expression level of two or more, three or more, four or more, five or more, or six or more of PGK1, NQO1, HMOX1, VEGFA, ADM, HPCAL1, PLK3, FOSL1, and PLAU to the reference expression level of two or more, three or more, four or more, five or more, or six or more of PGK1, NQO1, HMOX1, VEGFA, ADM, HPCAL1, PLK3, FOSL1, and PLAU indicates higher expression level of two or more, three or more, four or more, five or more, or six or more of PGK1, NQO1,HMOX1, VEGFA, ADM, HPCAL1, PLK3, FOSL1, and PLAU in the sample, indicating that the subject has an increased risk of short survival and/or recurrence of breast cancer. Suitable statistical and other analysis can be carried out to confirm a change (e.g., an increase or a higher level of expression) in one or more of PGK1, NQO1,HMOX1, VEGFA, ADM, HPCAL1, PLK3, FOSL1, and PLAU when compared with a reference sample.

The gene expression panel or array can consist of primers or probes capable of detecting, amplifying or otherwise measuring the presence or expression of one or more genes disclosed herein. For example, specific primers that can be used in the methods disclosed herein include, but are not limited to the primers suitable for use in the standard exon array from the Affymetrix website listed at: http://www.affYmetrix.com. In some aspects, the gene expression panel or array disclosed herein for can be used to identify determine or assess the risk of short survival and/or recurrence of glioblastoma in a subject, wherein DUSP5, PLK3, PPPIR15A, FOSL1, CDKNIA, KLF6, VDR, ARL4C, ADM, PLAU, VEGFA, NQO1, HMOX1, PGK1, LITAF, HPCAL1 and FTH1 RNA expression levels are detected in the sample. In some aspects, the gene expression panel or array disclosed herein for can be used to identify determine or assess the risk of short survival and/or recurrence of glioblastoma in a subject, wherein PGK1, NQO1,HMOX1, VEGFA, ADM, HPCAL1, PLK3, FOSL1, and PLAU expression levels are detected in the sample. In some aspects, the gene expression panel or array disclosed herein for can be used to identify determine or assess the risk of short survival and/or recurrence of breast cancer in a subject, wherein PGK1, NQO1, HMOX1, VEGFA, ADM, HPCAL1, PLK3, FOSL1, and PLAU expression levels are detected in the sample.

In some aspects, a diagnostics kit is disclosed comprising one or more probes or primers capable of detecting, amplifying or measuring the presence or expression of one or more genes disclosed herein.

Disclosed herein, are solid supports comprising one or more primers, probes, polypeptides, or antibodies capable of hybridizing or binding to one or more of the genes disclosed herein. Solid supports are solid state substrates or supports that molecules, such as analytes and analyte binding molecules, can be associated. Analytes (e.g., calcifying nanoparticles and proteins) can be associated with solid supports directly or indirectly. For example, analytes can be directly immobilized on solid supports. Analyte capture agents (e.g., capture compounds) can also be immobilized on solid supports.

As mentioned above, one of ordinary skill in the art can determine the expression level of one or more genes (or proteins) disclosed herein any number of ways. To detect or quantify the level of RNA products of the biomarkers within a sample, arrays, such as microarrays, RT-PCR (including quantitative RT-PCR), nuclease protection assays and Northern blot analyses can be used. Accordingly, in some aspects, the biomarker expression levels can be determined using arrays, microarrays, RT-PCR, quantitative RT-PCR, nuclease protection assays or Northem blot analyses.

An array is a form of solid support. An array detector is also a form of solid support to which multiple different capture compounds or detection compounds have been coupled in an array, grid, or other organized pattern.

Solid-state substrates for use in solid supports can include, for instance, any solid material to which molecules can be coupled. Examples of such materials include acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, poly lactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or any combination thereof. Solid-state substrates and solid supports can be porous or non-porous. An example of a solid-state substrate is a microtiter dish (e.g., a standard 96-well type). A multiwell glass slide can also be used. For example, such as one containing one array per well can be used, allowing for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array can generally have one type of component (that is, all the components at that location are the same). Each location can have multiple copies of the component. The spatial separation of different components in the array allows separate detection and identification of the polynucleotides or polypeptides disclosed herein.

It is not required that a given array be a single unit or structure. The set of compounds can be distributed over any number of solid supports. For example, each compound can be immobilized in a separate reaction tube or container, or on separate beads or microparticles. Different aspects of the disclosed method and use of the gene expression panel or array or diagnostic device can be performed with different components (e.g., different compounds specific for different proteins) immobilized on a solid support.

Some solid supports can have capture compounds, such as antibodies, attached to a solid-state substrate. Such capture compounds can be specific for calcifying nanoparticles or a protein on calcifying nanoparticles. Captured calcified nanoparticles or proteins can then be detected by binding of a second detection compound, such as an antibody. The detection compound can be specific for the same or a different protein on the calcifying nanoparticle.

Methods for immobilizing nucleic acids, peptides or antibodies (and other proteins) to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidinbiotin, photocrosslinkable agents, epoxides, maleimides and N-[y-Maleimidobutyryloxy] succinimide ester (GMBS), and a heterobifunctional crosslinker. Antibodies can be attached to a substrate by chemically cross-linking a free amino group on the antibody to reactive side groups present within the solid-state substrate. Antibodies can be, for example, chemically cross-linked to a substrate that contains free amino, carboxyl, or sulfur groups using glutaraldehyde, carbodiimides, or GMBS, respectively, as cross-linker agents. In this method, aqueous solutions containing free antibodies can be incubated with the solid-state substrate in the presence of glutaraldehyde or carbodiimide.

A method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS3) or a heterobifunctional cross-linker agent such as GMBS. For crosslinking with GMBS, glass substrates can be chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides can be activated by immersing in a 0.5 mg/ml solution of GMBS in 1% dimethylformamide, 99% ethanol for 1 hour at room temperature. Antibodies or proteins can be added directly to the activated substrate, which can be blocked with solutions containing agents such as 2% bovine serum albumin, and air-dried. Other standard immobilization chemistries are known by those of ordinary skill in the art.

Each of the components (e.g., compounds) immobilized on the solid support can be located in a different predefined region of the solid support. Each of the different predefined regions can be physically separated from each other. The distance between the different predefined regions of the solid support can be either fixed or variable. For example, in an array, each of the components can be arranged at fixed distances from each other, while components associated with beads will not be in a fixed spatial relationship. The use of multiple solid support units (e.g., multiple beads) can result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

In addition, the genes described herein can also be used as markers (i.e., biomarkers) for risk of short survival and/or recurrence of glioblastoma or presence or progression of glioblastoma. The methods and assays described herein can be performed over time, and the change in the level of the markers assessed. For example, the assays can be performed every 24-72 hours for a period of 6 months to 1 year, and thereafter carried out as needed. Assays can also be completed prior to, during, or after a treatment protocol. Together, the genes disclosed herein can be used to profile an individual’s risk or progression of recurrence of glioblastoma. As used within this context, the terms “differentially expressed” or “differential expression” refers to difference in the level of expression of the biomarkers disclosed herein that can be assayed by measuring the level of expression of the products (e.g., RNA or gene product) of the biomarkers, such as the difference in level of messenger RNA transcript or a portion thereof expressed or of proteins expressed of the biomarkers. In some aspects, this difference is significantly different.

To improve sensitivity, more than one gene disclosed herein can be assayed within a given sample. Binding agents specific for different proteins, antibodies, nucleic acids provided herein can be combined within a single assay. Further, multiple primers or probes can be used concurrently. To assist with such assays, specific biomarkers can assist in the specificity of such tests.

Levels of expression can be measured at the transcriptional and/or translational levels. At the translational level, expression of any of the genes described herein can be measured using immunoassays including immunohistochemical staining, western blotting, ELISA and the like with an antibody that selectively binds to the corresponding gene or a fragment thereof. Detection of the protein using protein-specific antibodies in immunoassays is known in the art. At the transcriptional level, mRNA can be detected by, for example, amplification (e.g., PCR, LCR), or hybridization assays (e.g., northern hybridization, RNAse protection, or dot blotting). The level of protein or mRNA can be detected, for example, by using directly or indirectly labeled detection agents (e.g., fluorescently or radioactively labeled nucleic acids, radioactively or enzymatically labeled antibodies). Changes (e.g., increase or decrease) in the transcriptional levels can also be measured using promoter-reporter gene fusion constructs. For example, the promoter region of a gene encoding any of the genes disclosed herein can be fused (i.e., operably linked) to the coding sequence of a polypeptide that produces a detectable signal. Reporter constructs are well known in the art. Examples of reporter sequences include fluorescent proteins (e.g., green, red, yellow), phosphorescent proteins (e.g., luciferase), antibiotic resistance proteins (e.g., beta lactamase), enzymes (e.g., alkaline phosphatase).

### EXAMPLES

### Example 1: Predicting Progression-Free Survival and Recurrence Time of Primary Glioblastoma Patients with a Microfluidic Assay for Quantification of Cell Invasion (MAqCI)

Abstract. Glioblastoma (GBM) is the most aggressive form of brain cancer characterized by high recurrence and dismal prognosis. Presently, there is no effective in vitro platform that can rapidly measure complex cellular phenotypic traits and predict patient-specific clinical outcomes. Here, an in vitro testing platform was fabricated and evaluated, Microfluidic Assay for Quantification of Cell Invasion (MAqCI), by screening a panel of 28 patient-derived primary GBM specimens in a blinded multi-institutional retrospective cohort study. The ability of GBM cells to navigate and squeeze through confined microenvironments that mimic tight perivascular conduits and white matter tracts in the brain parenchyma in vivo, as well as the proliferative capacity of highly motile subpopulations was quantified. By combining migratory- and proliferative-based indices, MAqCI categorized patients into short- or long-term survival groups with high sensitivity (84%), specificity (89%), and accuracy (86%), and predicted time to recurrence. In addition, MAqCI classified the 5 patients tested accurately based on their survival outcomes. RNA sequencing of isolated highly motile cells identified differentially expressed genes that correlate with poor prognosis in GBM patients. Overall, the findings described herein suggest that invasive growth is intimately linked with GBM progression and patient outcomes, and reveals the translational potential of MAqCI for personalized GBM care.

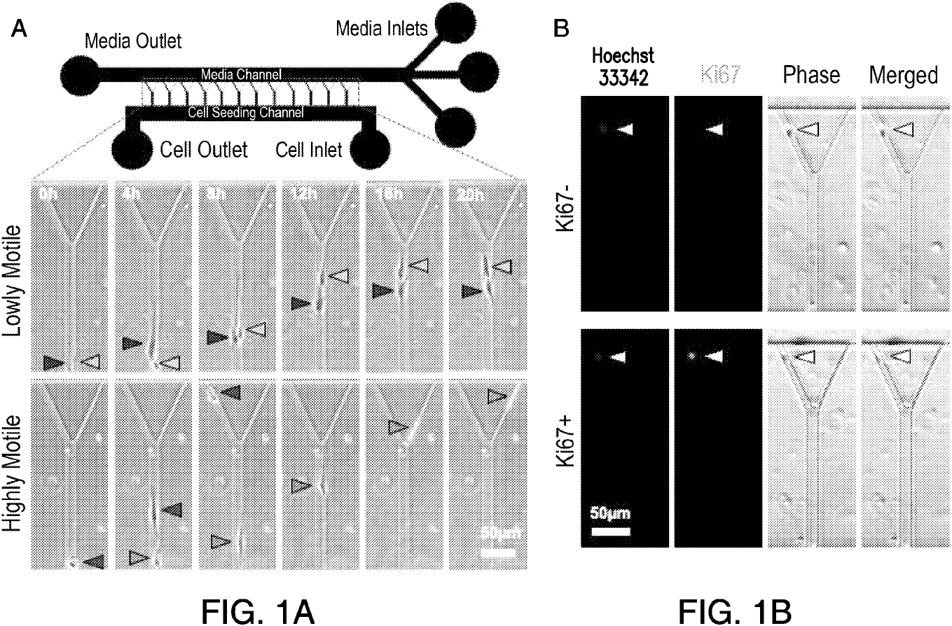

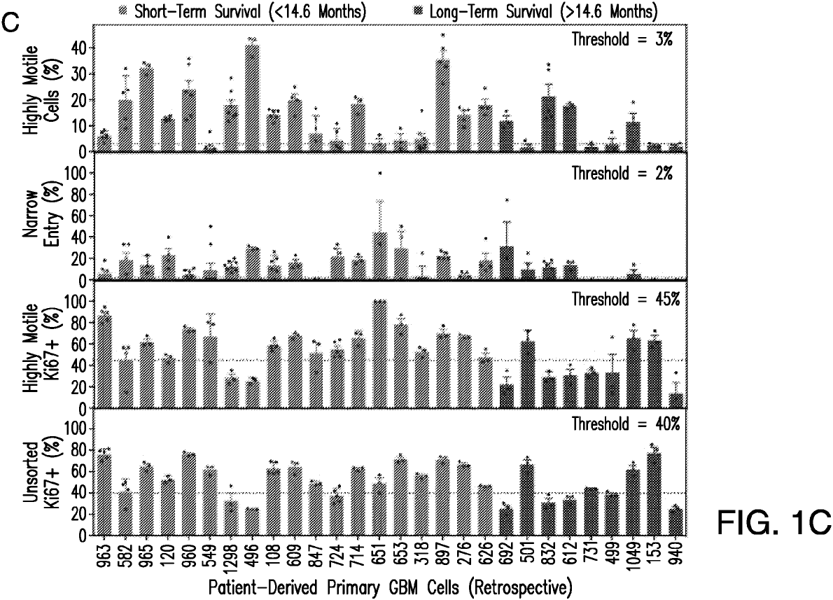

As disclosed herein, the MAqCI technology can be utilized to concurrently evaluate the migratory and proliferative potentials of patient-derived primary GBM specimens. MAqCI consists of two parallel seeding and media channels connected by a series of 10 µm-high Y-shaped microchannels (Paul, C. D. et al., FASEB J 30, 2161-2170, (2016)) with 20 µm-wide feeder channels bifurcating into 10 µm- and 3 µm-wide branches (FIG. 1A). These microchannels aim to recapitulate aspects of the complex topography and confining longitudinal pores or perivascular tracks of the brain parenchyma formed between glial cells and the basement membrane of vascular smooth muscle cells which ranges from 10-300 µm (Stupp, R. et al., N Engl J Med 352, 987-996 (2005)) in diameter (Wolf, K. et al., Semin Cell Dev Biol 20, 931-941 (2009)). By quantifying the relative abundance of highly motile cells that have successfully traversed the Y-shape microchannels as well as the proliferative potential of this migratory subpopulation, the results described herein demonstrate that MAqCI predicted individual patient survival and time to recurrence with high sensitivity, specificity and accuracy in a retrospective patient cohort. Furthermore, MAqCI classified the patients accurately based on their survival outcomes. RNA sequencing of isolated highly motile was compared to unsorted bulk GBM cells and revealed a group of differentially expressed genes (DEGs) whose individual expression patterns matched those of GBM patients with reduced overall survival. In sum, the results disclosed herein suggest that invasive spread and tumor growth are primary hallmarks of tumor aggressiveness which can be exploited to accurately predict patient clinical outcomes.

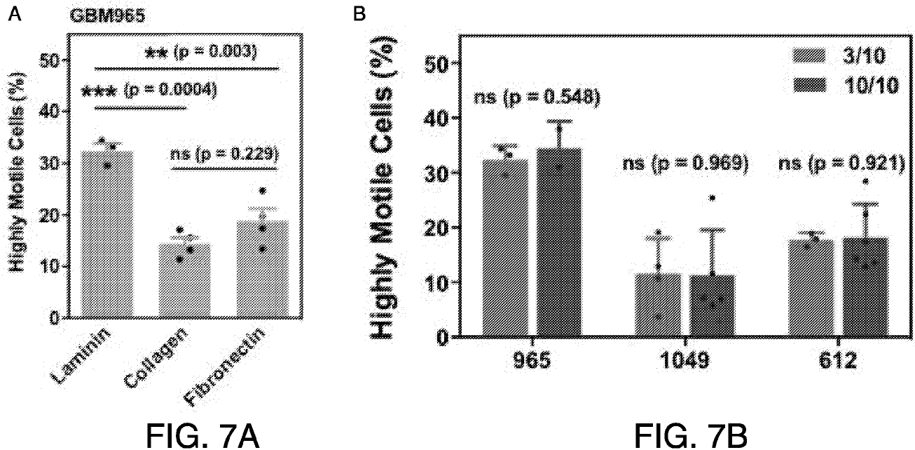

Results. MAqCI distinguishes patient-derived primary GBM cells based on their migratory and proliferative potentials. To assess the capacity of MAqCI to predict individual GBM patient outcomes, the migratory and proliferative potentials of patient-derived primary GBM cells were evaluated. The cells that enter the feeder channels were analyzed and classified into 2 categories based on their migratory behaviors: lowly motile cells are defined as cells that migrated into the feeder channels, but failed to reach and/or enter the bifurcations, while highly motile cells are defined as cells that successfully traversed the entire length of the feeder channels and entered either one of the branch channels (FIG. 1A). Using live-cell imaging, the percentage of highly motile cells that migrated in the microchannels, and the percentage of these highly motile cells that preferentially entered the narrower 3 µm-wide branches (termed as percentage of narrow entry) was calculated. Laminin was used to coat MAqCI as it represents the most abundant extracellular matrix found in brain microenvironment, (Chintala, S. K., et al., Front Biosci 1, d324-339 (1996)) and promoted the most efficient migration in the patient-derived primary GBM cells over other substrates, such as collagen and fibronectin (FIG. 7A). The widths of the microchannels in MAqCI ranged from 3 to 20 µm to recapitulate the relevant dimensions of diverse in vivo pre-existing tissue tracks as measured by intravital microscopy (Weigelin, B. et al., Intravital 1, 32-43 (2012)). The 3 µm channels were created specifically to evaluate the ability for GBM cells to deform their cytoskeleton and nucleus to enter into tight constrictions. It was tested whether the ability for cells to deform and migrate in confining microenvironment could correlate with their aggressiveness and invasiveness. Interestingly, the same values of percentage of highly motile cells in symmetrical 10/10 µm branch channels and the 3/10 µm asymmetrical design (FIG. 7B) was observed. This result suggested that a symmetrical branch provides identical information as the asymmetrical design in terms of the percentage of highly motile cells, but less information in terms of the cell’s ability to deform. Thus, the 3/10 µm asymmetrical design was utilized to predict GBM patient outcomes.

Aside from cell migration, cell proliferation is also an important factor that governs cancer aggressiveness and ability to colonize (Xie, Q et al., Neuro Oncol 16, 1575-1584 (2014)). Ki67 is a nuclear antigen that is specific to actively proliferating cells and has been used in the clinic to evaluate cancer patient prognosis (Inwald, E. C. et al., Breast Cancer Research and Treatment 139, 539-552 (2013)). Using MAqCI, the ability to assess the proliferative capacities of GBMs, either for the unsorted bulk population or specifically for the sorted highly motile subpopulation was assessed. Actively proliferating cells stain positive for Ki67 (FIG. 1B). The percentages of Ki67-positive highly motile or unsorted cells were quantified.

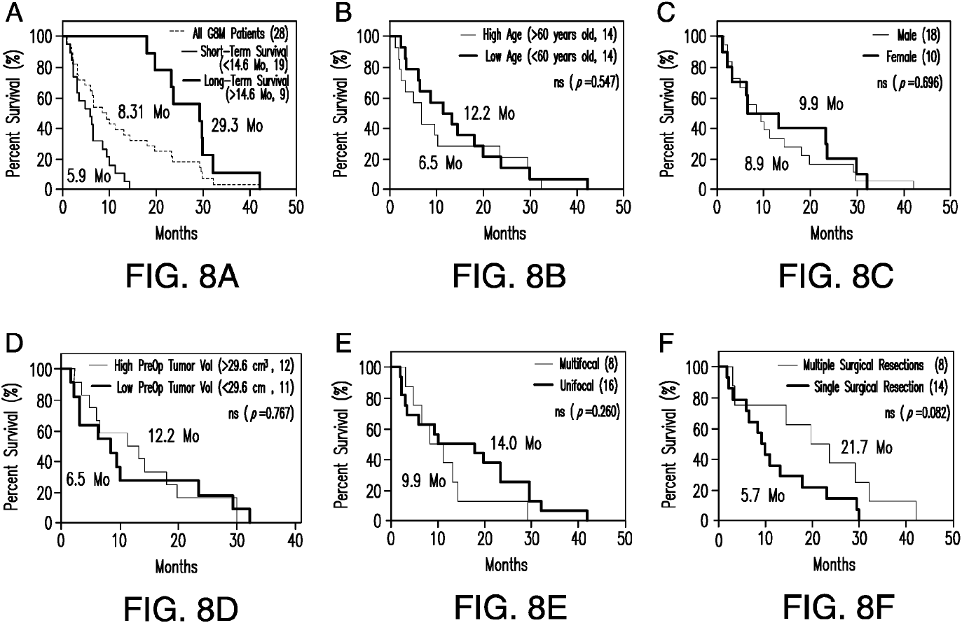

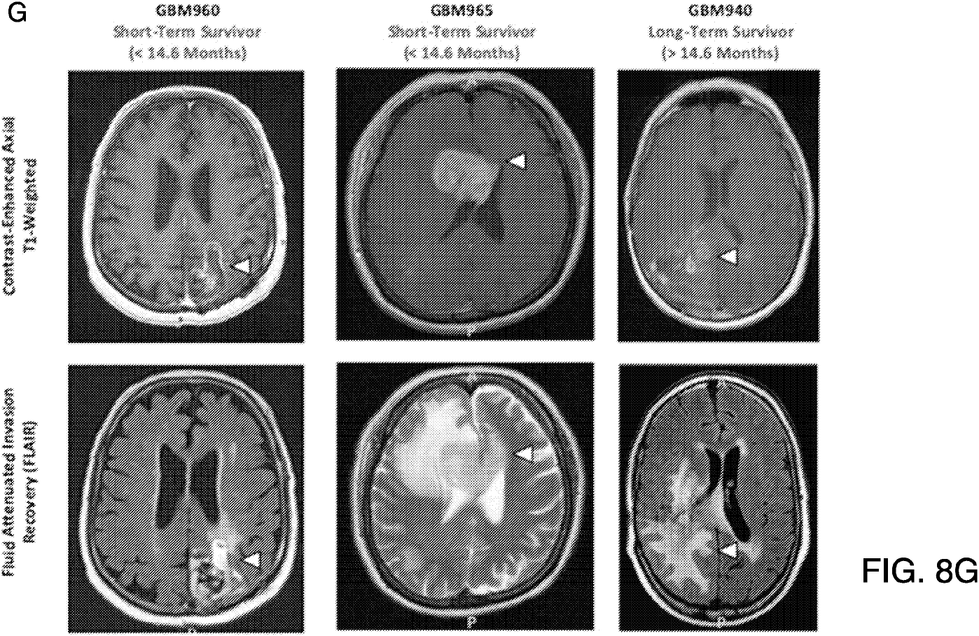

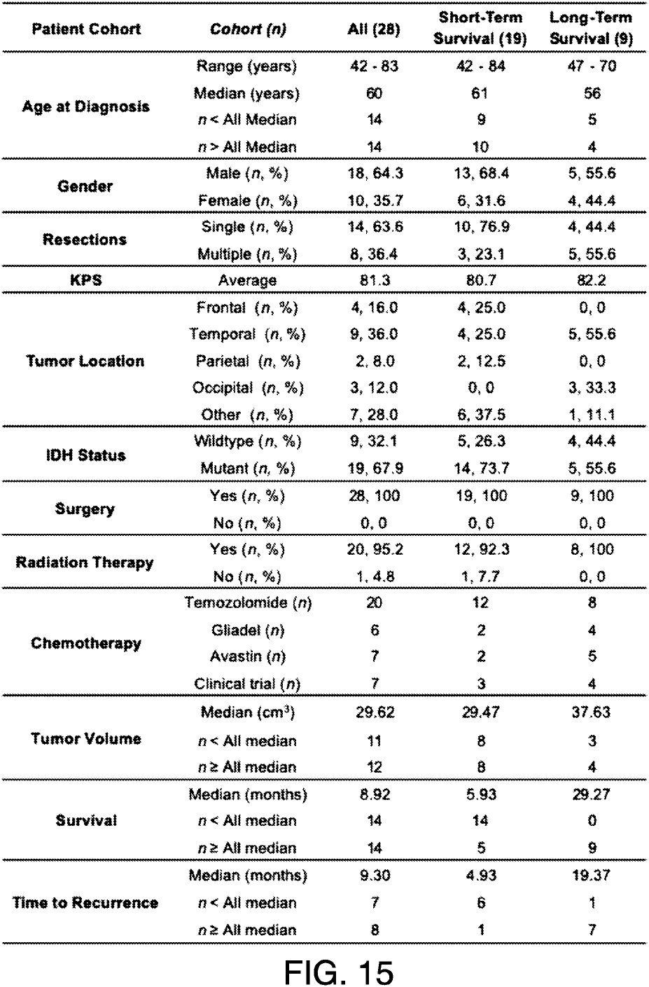

A retrospective panel of 28 patient-derived primary GBM cells with complete clinical outcome information was tested in MAqCI in a blind manner to assess their percentages of highly motile cells, narrow entry, highly motile Ki67-positive cells and unsorted Ki67-positve cells. These cells exhibited a heterogeneous and wide range of migratory patterns and proliferative potentials (FIG. 1C). The median survival of this retrospective population was 8.9 months and consisted of 19 patients whom are classified as short-term survivors (<14.6 months, median=5.9 months) and 9 patients whom are classified as long-term survivors (>14.6 months, median=29.3 months) based on the 14.6 months of median GBM patient survival threshold established by Stupp et al. (Stupp, R. et al., N Engl J Med 352, 987-996, (2005)) (FIG. 8A). A list of the relevant and available demographic, tumor, surgical and clinical characteristics for the entire retrospective cohort, as well as the 2 survival subgroups (i.e., short- versus long-term), is available in FIG. 15. Notably, these demographic (age and gender), tumor (pre-operative tumor volume and tumor spread) and surgical attributes (number of surgical resections) failed to correlate with GBM patient survival (FIGS. 8B-F). Representative magnetic resonance imaging of the brains of GBM patients reveals a variable degree of tumor size and invasive spread (FIG. 8G). Generally, GBM patients with short-term survival possess a larger degree of butterfly tumor spread than long-term survivors, alluding to the potential roles of migration and proliferation as determinants of patient prognosis, (Shah, S. R. et al., bioRxiv, 602052 (2019)). However, this pattern was not consistent across our retrospective cohort. Additionally, while the extent of resection and residual tumor volume have correlated with patient prognosis (Chaichana, K. L. et al, Neuro Oncol 16, 113-122 (2014)), leading surgical teams to aim for gross total resection, pre-operative tumor size has not robustly informed patient outcomes. Given that tumor size is variable during initial presentation at the time of diagnosis, radiographic measures alone cannot be reliably used to predict patient survival. Thus, it is important to utilize other measures to accurately evaluate clinical outcomes for each GBM patient.

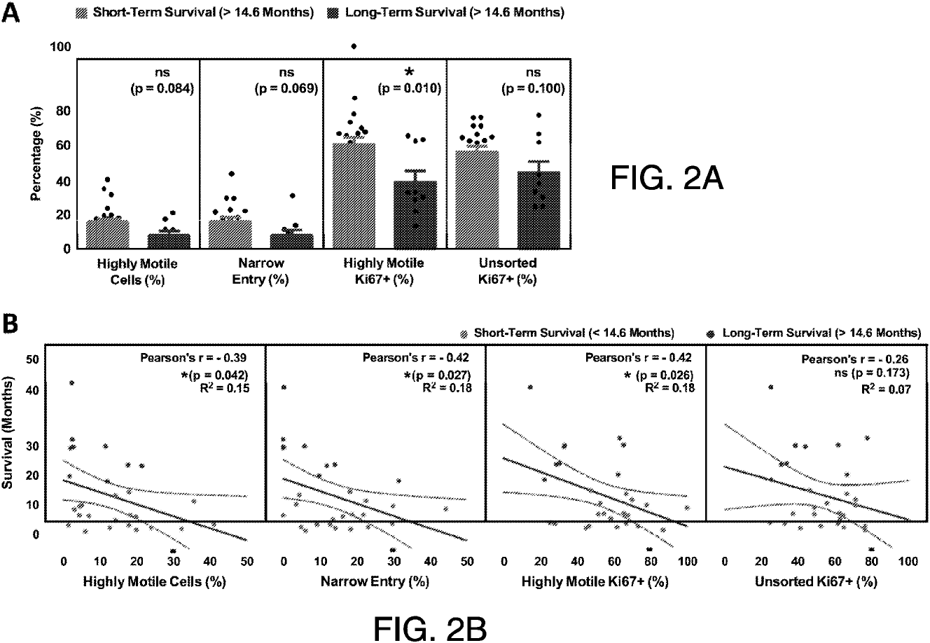

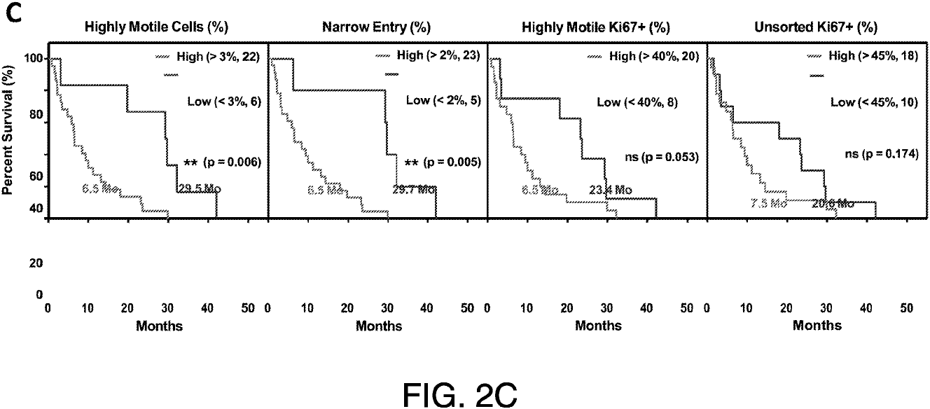

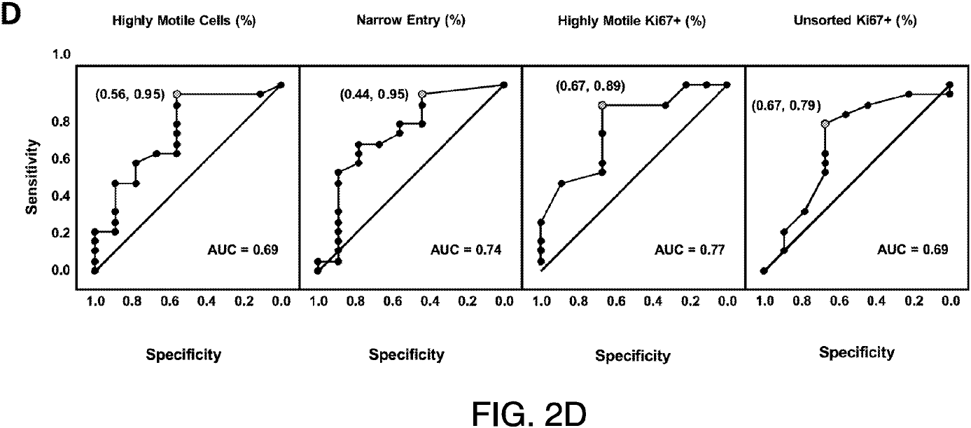

Migratory and proliferative potentials of GBMs correlate with GBM patient survival. To begin evaluating the prognostic values of the various MAqCI measurement metrics, the retrospective patients were separated into either short- or long-term survival groups, and their percentages of highly motile cells, narrow entry, highly motile Ki67-positive cells and unsorted Ki67-positive cells were compared. Interestingly, primary GBM cells that are derived from the short-term survivor cohort displayed higher migratory and proliferative potentials, as evidenced by their significantly higher percentage of highly motile Ki67-positive cells (FIG. 2A). The short-term survival group also exhibited a trend of higher percentages of highly motile cells, narrow entry and unsorted Ki67-positive cells (FIG. 2A).

Linear regression between each of the MAqCI measurement metrics and GBM patient survival in months revealed that percentages of highly motile cells, narrow entry and highly motile Ki67- positive cells were significantly negatively correlated with GBM patient survival (FIG. 2B). Interestingly, there was no significant correlation between percentage of unsorted Ki67-positive cells and GBM patient survival (FIG. 2B).

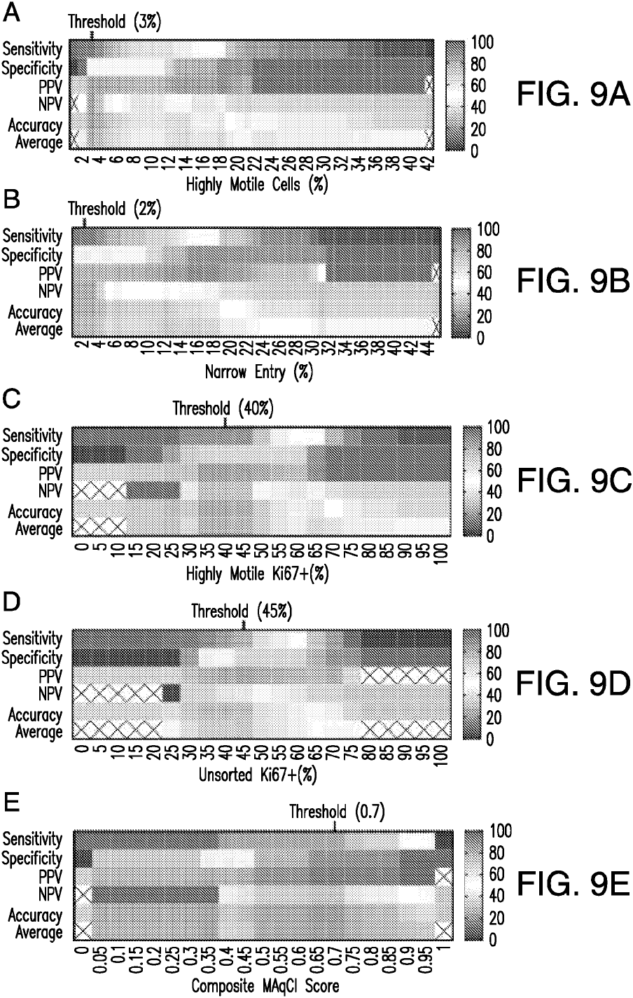

Next, a threshold value was determined for each MAqCI measurement metric that can be used to classify the retrospective cohort into either short- or long-term survival groups based on the 14.6 months of median GBM survival threshold established by Stupp et al. (Stupp, R. et al., N Engl J Med 352, 987-996 (2005)). The individual thresholds used to separate the patients were determined at levels that optimized the sensitivity, specificity, positive predictive value (PPV), negative predictive value (NPV) and accuracy of MAqCI to correctly categorize GBM patients into either short- or long-term survivors (FIGS. 9A-D). Segmenting the 28 retrospective patients based on the optimal threshold percentages of highly motile cells (3%) or narrow entry (2%) achieved a significant separation of the Kaplan-Meier survival curves, with the curves corresponding to the group exceeding the threshold (high) exhibiting a significantly shorter median survival months than the group that fell below the threshold (low) (FIG. 2C). The optimal threshold percentage of highly motile Ki67-positive cells (40%) unveiled a trend of separating the survival curves, while the percentage of unsorted Ki67-positive cells (45%), failed to achieve significant survival curve separation (FIG. 2C).

Lastly, receiver-operating characteristic (ROC) curves and their corresponding area under curve (AUC) were generated and calculated for each MAqCI measurement metric to quantify their ability to correctly identify patients based on their actual survival outcomes (FIG. 2D). ROC curve is a graphical representation of the benefit-cost tradeoff between the true positive (i.e., sensitivity) and false positive (i.e., 1 minus specificity) of a binary classifier system as its discriminatory threshold is systematically varied. The AUC of ROC indicates the usefulness of a test, where a higher value (with a maximum of 1) corresponds to a more useful test. In addition, the sensitivity, specificity, PPV, NPV and accuracy for each

MAqCI measurement metric at their optimal threshold were also tabulated in Table 1. In general, the individual MAqCI measurement metrics were able to achieve similar values of around 90% sensitivity and 80% accuracy (Table 1). It is noteworthy that the percentage of unsorted Ki67-positive cells represented the most inferior discriminators as compared to the other 3 metrics as it had the lowest value of accuracy (Table 1) and AUC of ROC (FIG. 2D), and did not show any linear correlation to GBM patient survival (FIG. 2B) and significant Kaplan-Meier survival curve separation (FIG. 2C).

Table 1 below measures of performance for individual MAqCI measurement metrics and composite MAqCI score in classifying the retrospective GBM patient cohort into either short- or long-term survivors. A positive event is defined as short-term survival (<14.6 months) while a negative event is defined as long-term survival (>14.6 months). Sensitivity is defined as the probability of correctly identifying a short-term survival patient from the short-term survival patients. Specificity is defined as the probability of correctly identifying a long-term survival patient from the long-term survival patients. Positive predictive value (PPV) is defined as the proportion of patients who are predicted to be short-term survival that are truly the short-term survivors. Negative predictive value (NPV) is defined as the proportion of patients who are predicted to be long-term survival that are truly the long-term survivors. Accuracy is defined as the probability of correctly identifying both the short- and long-term survivors from the entire population. Area under curve (AUC) is defined as the area under the receiver operating characteristic curve (ranges from 0 to 1). AUC measures how capable each classifier is able to distinguish and separate the short-term from the long-term GBM survival groups.

TABLE 1

Individual MAqCI measurement metrics and composite MAqCI score in classifying the retrospective GBM patient cohort into either short- or long-term survivors

| | Threshold | Sensitivity (%) | Specificity (%) | PPV (%) | NPV (%) | Accuracy (%) | AUC |
|---|---|---|---|---|---|---|---|
| Highly Motile Cells (%) | 3 | 94.7 | 55.6 | 81.8 | 83.3 | 82.1 | 0.69 |
| Narrow Entry (%) | 1-3 | 94.7 | 44.4 | 78.3 | 80.0 | 78.6 | 0.74 |
| Highly Motile Ki67+ (%) | 35-45 | 89.5 | 66.7 | 85.0 | 75.0 | 82.1 | 0.77 |
| Unsorted Ki67+ (%) | 45 | 78.9 | 66.7 | 83.3 | 60.0 | 75.0 | 0.69 |
| IDH1 Mutation Status | Yes/No | 26.3 | 55.6 | 55.6 | 26.3 | 34.5 | 0.41 |
| Cell Index (at 24h) | 0.15 | 5.6 | 88.9 | 50.0 | 32.0 | 33.3 | 0.28 |
| Composite MAqCl Score | 0.65-0.70 | 84.2 | 88.9 | 94.1 | 72.7 | 85.7 | 0.87 |

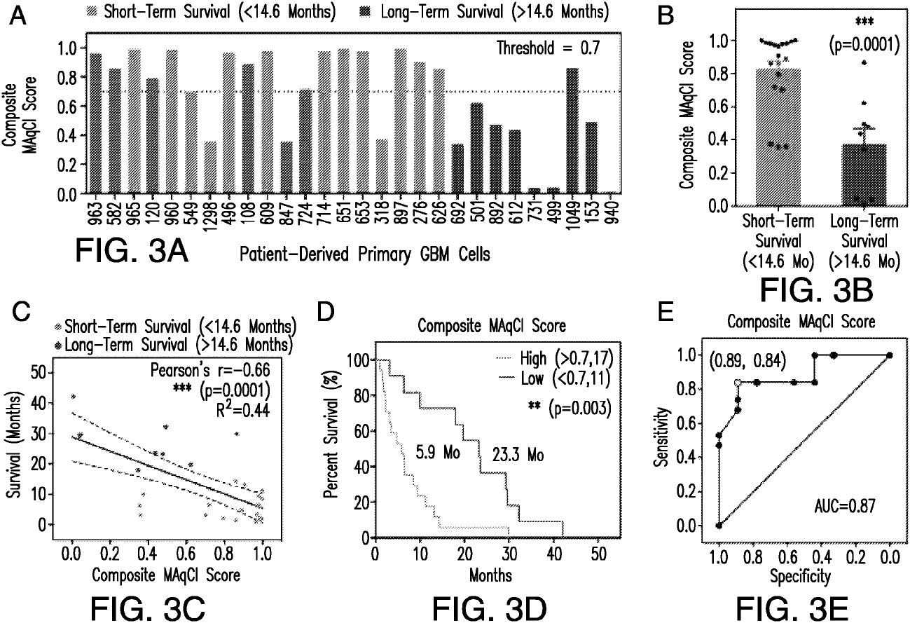

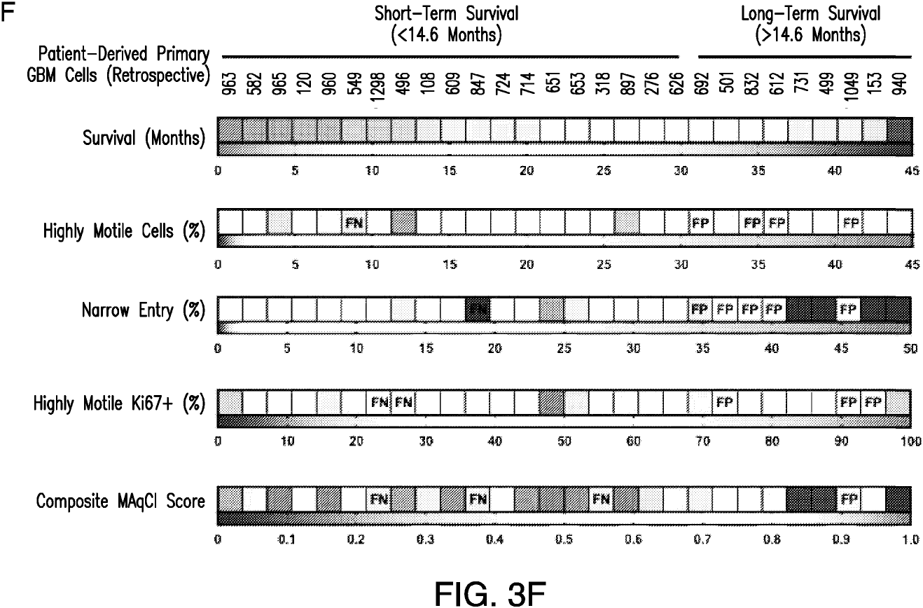

Combining the migratory and proliferative indices into a single composite score maximizes the prognostic performance of MAqCI. The percentages of highly motile cells, narrow entry and highly motile Ki67-positive cells correlated to GBM patient survival, but exhibited a rather suboptimal prognostic capability compared to what is considered to be clinically useful. In order to further improve the predictive power of MAqCI, these 3 indices were combined into a composite MAqCI score (ranging from 0 to 1) using logistic regression (FIG. 3A, FIG. 16). A composite MAqCI threshold score of 0.7 was used to stratify patients into high (>0.7, n=17) and low composite MAqCI score groups (<0.7, n=11), respectively (FIG. 9E). The differences in the average composite MAqCI score between the short- and long-term survivors were magnified, with short-term survivors having a significantly higher composite MAqCI score than the long-term survivors (FIG. 3B). The linear correlation between composite MAqCI score and GBM patient survival in months also improved substantially with a noticeably higher R2 value (FIG. 3C). This classification was able to achieve statistically significant Kaplan-Meier survival curves separation (FIG. 3D). Importantly, the sensitivity, specificity, PPV and NPV and accuracy of employing composite MAqCI score to correctly identify short- and long-term survival patients markedly improved to approximately 90% (Table 1). Finally, the AUC of ROC of the composite MAqCI score was also increased to close to 0.90 from around 0.80 for the individual MAqCI measurement metrics, signifying that the composite MAqCI score is a superior binary discriminator than the individual MAqCI measurement metrics (FIG. 3E).

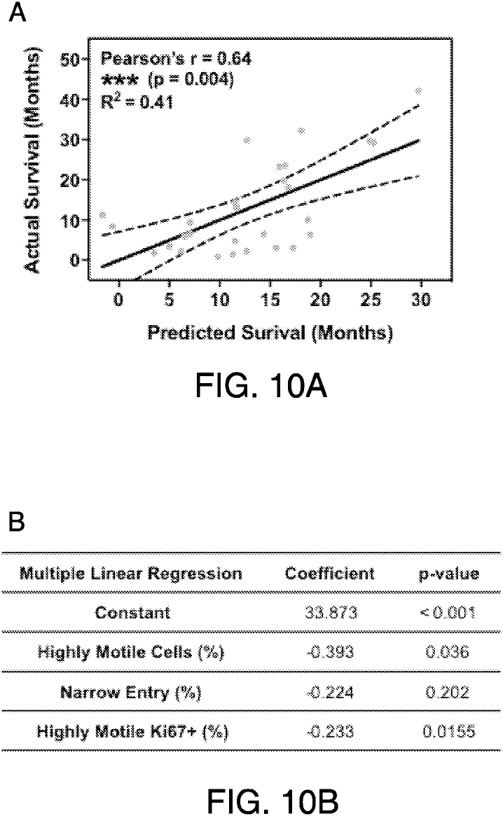

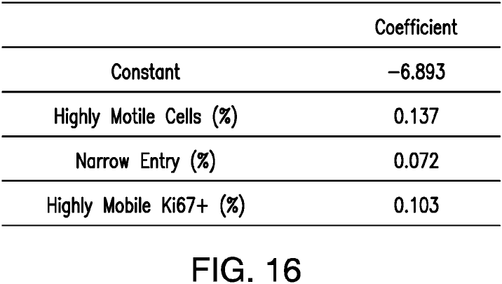

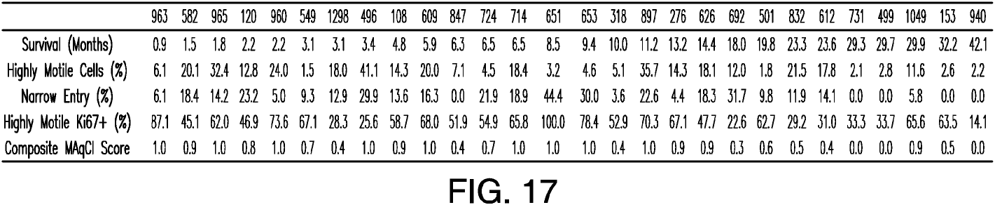

The ability for each individual MAqCI measurement metric and the composite MAqCI score to categorize the 28 retrospective GBM patients into short- versus long-term survival cohorts is summarized in a heat map (FIG. 3F). The numerical individual values behind the heat map classification is provided in FIG. 17. The patient-derived primary GBM lines were arranged in order of increasing survival and color coded with a red-blue double gradient with white color set at the threshold of 14.6 months, while the true red and true blue colors represent the shortest- and longest-term survival, respectively. The effectiveness of the individual MAqCI measurement metrics and the composite MAqCI score were also represented in the red-blue double gradient with white color being set as the optimal threshold previously determined (i.e., 3% highly motile cells, 2% narrow entry, 40% highly motile Ki67-positive cells, 0.7 composite MAqCI score), and the true red and true blue color represents the highest and lowest value of each discriminators, respectively. With these heat maps, the false positive (i.e., patients who were long- term survivors, but incorrectly predicted as short-term survivors) and the false negative (i.e., patients who were short-term survivors, but incorrectly predicted as long-term survivors) results were identified by a mismatch in color hue to the survival heat map panel. The composite MAqCI score emerged as the most accurate binary discriminator compared to the individual MAqCI measurement metrics as it produced the least number of false positive and false negative predictions (FIG. 3F). In an attempt to achieve a quantitative prediction of survival time, a multiple linear regression analysis was also performed to generate a semiquantitative correlation between survival months, as treated as a continuous variable, and the various MAqCI measurement metrics. Interestingly, these results matched the findings based on binary classification using logistic regression. Notably, a significant positive correlation was observed between the predicted survival time (as calculated based on the coefficients of multiple linear regression) and actual patient survival time of a R2 value of 0.41 (FIGS. 10A-B). While this analysis suggests that MAqCI can provide semiquantitative prediction of survival time for each patient, this data strongly argues for use of a binary classification to inform patient outcomes. However, future development of this technology may provide better quantitative read-outs with increased statistical confidence. Additionally, separating the patient cohort into more than 2 survival groups or creating additional subgroups is challenging because there is no appropriate available clinical or literature guidance to support additional specific survival cutoff points for GBM patients that are meaningful. Along these lines, because less than 5% of GBM patients survive past 5 years (Ostrom, Q. T. et al., Neuro Oncol 20, iv1-iv86, doi:10.1093/neuonc/noy131 (2018)), and Stupp, R. et al., N Engl J Med 352, 987-996, (2005)), it is difficult to develop more than 2 clinically meaningful and distinct subgroups.

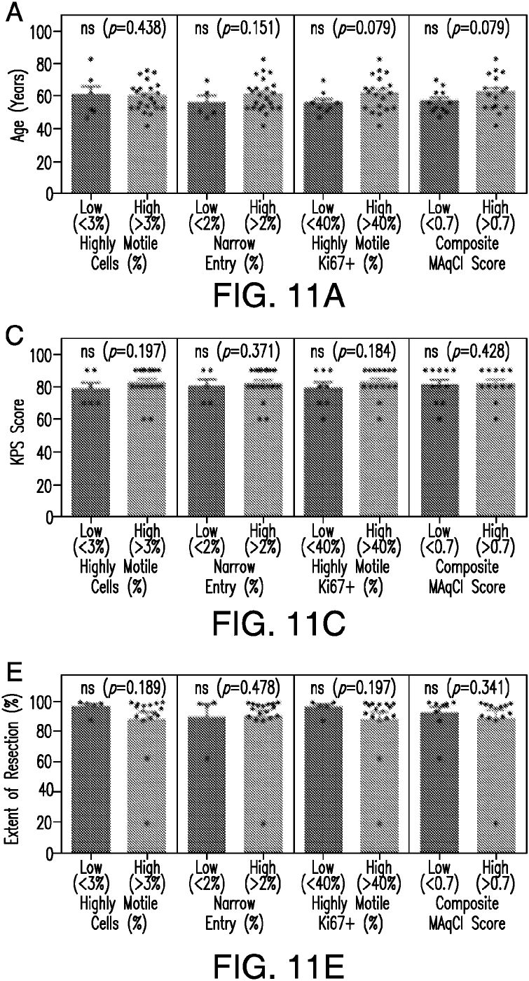

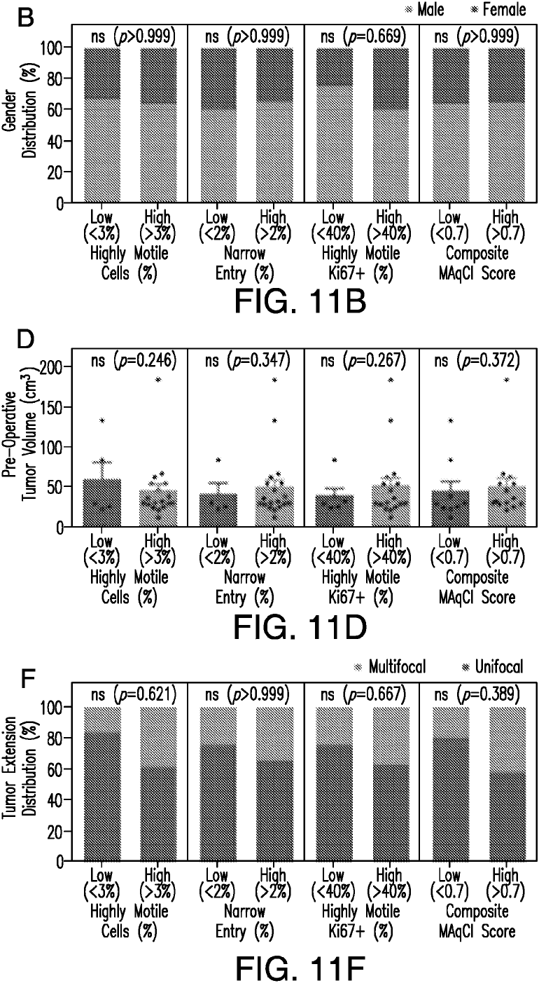

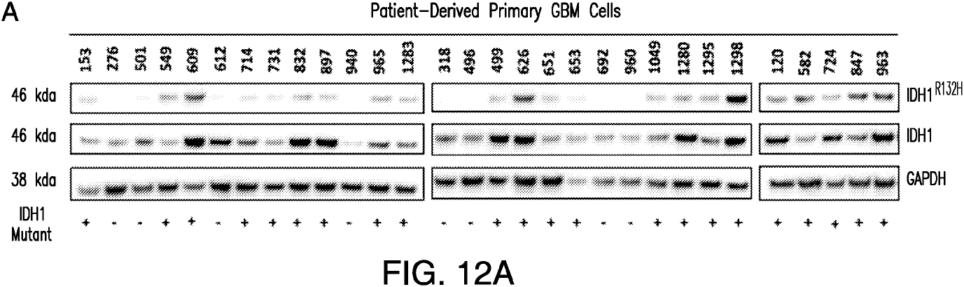

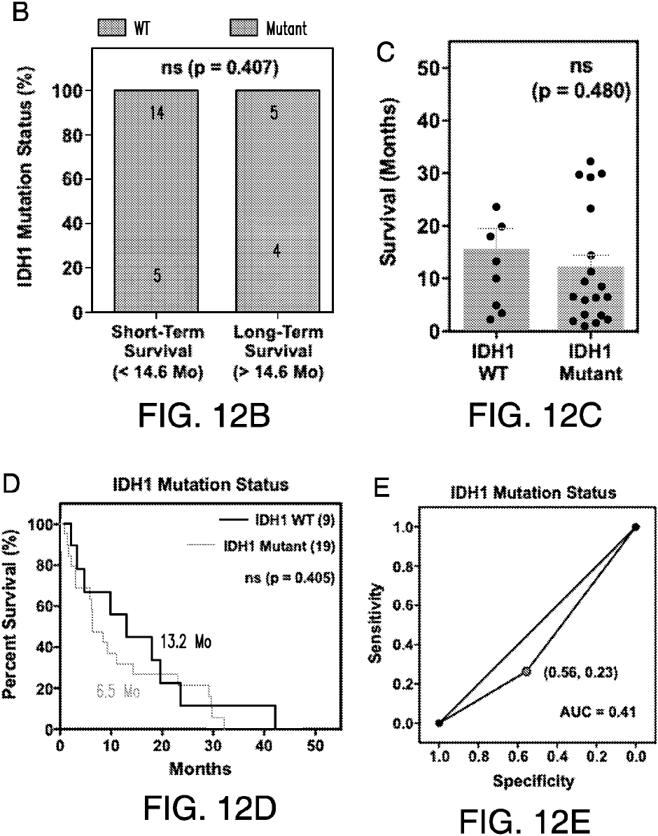

Notably, the survival prediction made by MAqCI is independent of the demographics, surgical, tumor and clinical attributes of the retrospective patient cohorts. There were no significant differences in terms of age, gender, KPS score, pre-operative tumor volume, extent of resection and tumor extension when the patients are separated based on low versus high MAqCI measurement metrics or composite MAqCI score with their optimized threshold (FIGS. 11A-F). While IDH1 mutation status has been shown to be an independent prognostic predictor for lower grade gliomas (Hartmann, C. et al., Acta Neuropathol 118, 469-474, (2009)), screening of the IDH1 mutation status of the retrospective patient cohort (FIG. 12A) revealed its shortcomings in predicting the survival of primary GBM patients. Short- and long-term survivors exhibited similar incidence of IDH1 mutation (FIG. 12B). There was also no difference in terms of mean or median progression-free survival (FIGS. 12C-D) of GBM patients harboring wild type or mutated IDH1. With an overall accuracy of 35% and an AUC of 0.41 ( FIG. 12E, Table 1), IDH1 mutation status possessed little utility in identifying patients based on their survival outcomes.

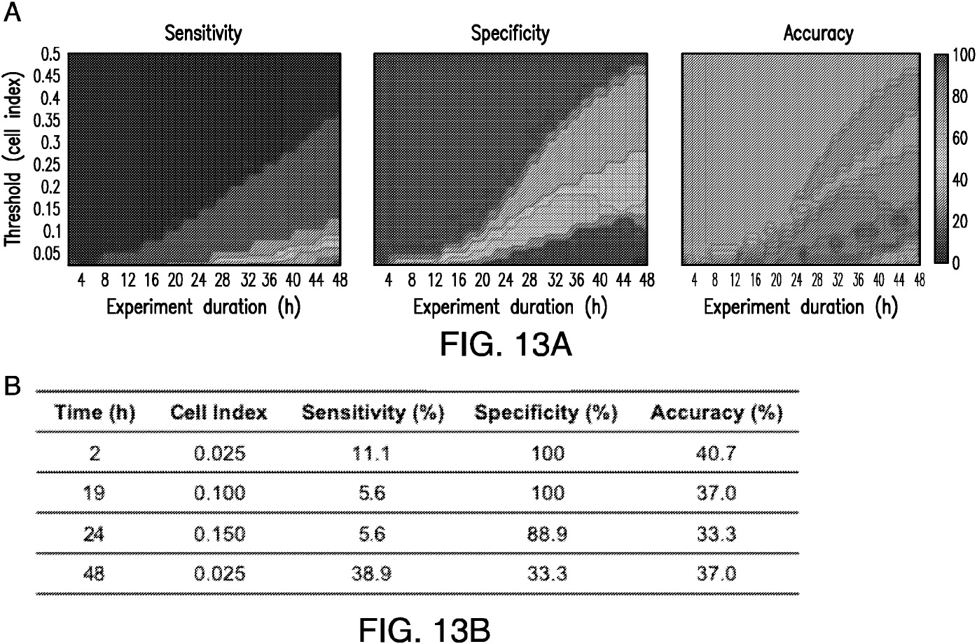

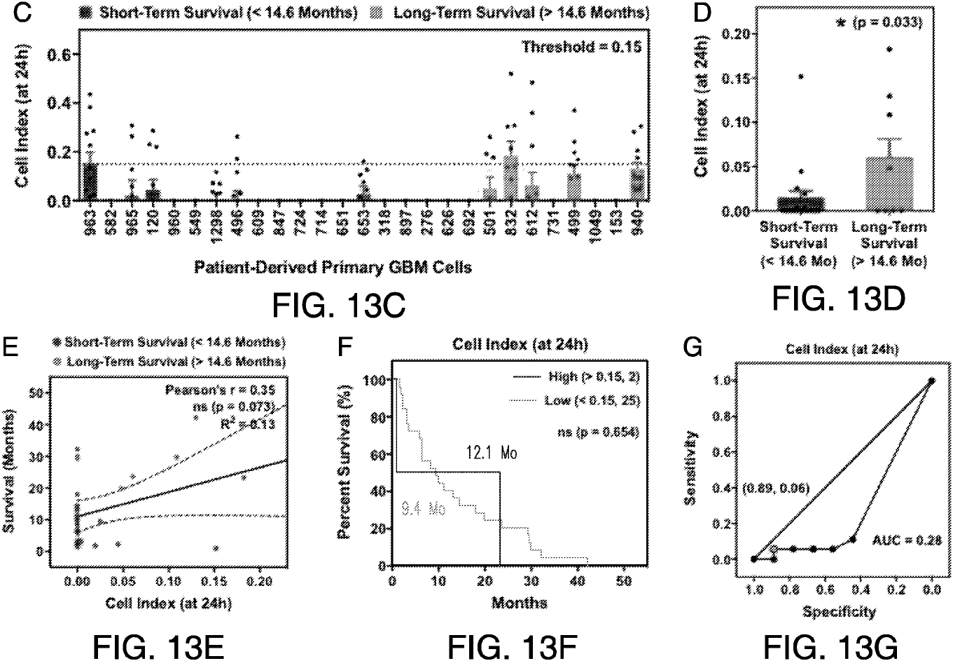

To directly compare the performance of MAqCI to that of conventional transwell migration assays for their ability to predict GBM patient survival, the xCELLigence RTCA DP instrument (Acea Biosciences, Inc.) was employed to monitor transwell-migration of the retrospective cohort in CIM-plate 16 chambers. These plates have chambers that are similar to Boyden chambers consisting of an upper chamber where the GBM cells are seeded in serum-free DMEM/F12, a microporous polyethylene terephthalate (PET) membrane with an average pore diameter of 8 µm (through which GBM cells migrate to the lower chamber), electrodes directly below this membrane, and a lower chamber which is filled with DMEM/F12 containing Gem21 Neuroplex supplemented with EGF and FGF as chemoattractants. The extent of GBM cell migration was monitored for 48 h at 15-min intervals by measuring changes in electrical impedance with electrodes that are attached directly underneath the PET membrane. Analysis of the transwell-migration assay revealed a markedly inferior ability to predict patient survival at comparable time scales to the MAqCI assay. Optimization of the sensitivity, specificity and accuracy of the transwell-migration assay by varying the experiment duration and cell index threshold (the readout of cell migration from this instrument) is shown in FIG. 13A. At the comparable timescale of MAqCI at 24 h, the accuracy and sensitivity of the transwell-migration assay were 33.3% and 5.6%, respectively (FIG. 13B, Table 1). At 24 h, most cells demonstrated extremely low levels of migration through transwell (FIG. 13C) and the long-term survival groups paradoxically exhibited a slightly higher cell index compared to the short-term survivors (Supplementary FIG. 13D). Nonetheless, there was no significant correlation between GBM patient survival in months (FIG. 13E) and Kaplan-Meier survival curve separation when the patients are categorized based on this transwell assay-derived cell index threshold (FIG. 13F), resulting in very low AUC of just 0.28 (FIG. 13G, Table 1). Taken together, these findings demonstrate the superiority of MAqCI over patient’s demographic, surgical, tumor and clinical attributes, IDH1 mutation status and conventional transwell-migration assay to accurately determine the survival outcomes of primary GBM in our retrospective patient cohort.

MAqCI predicts time to recurrence and GBM patient survival prospectively. Aside from GBM patient survival, MAqCI measurement metrics and the composite MAqCI score can also be used to predict time to recurrence. Time to recurrence in months is significantly negatively correlated to percentages of narrow entry and highly motile Ki67-positive cells, and composite MAqCI score, but not percentage of highly motile cells (FIG. 4A). However, GBM lines which were derived from patients with high percentages of highly motile cells, narrow entry and highly motile Ki67-positive cells, and composite MAqCI score had a significantly shorter time to recurrence (FIG. 4B).

To further evaluate the potential for MAqCI to be used in an actual clinical setting for GBM prognosis, MAqCI specimens were collected and tested from patients from two institutions (n=5). For this cohort, the patients were still alive at the time when the in vitro experiments and prediction analyses were conducted and hence the survival outcomes were not available. The percentages of highly motile cells, narrow entry and highly motile Ki67-positive cells were measured for these 5 prospective samples and computed the composite MAqCI score (FIG. 4C, Table 2). One out of the 5 samples (GBM1295) displayed a composite MAqCI score of >0.7 and was predicted to be a short-term survivor, while the other 4 samples (GBM1296, GBM1283, GBM1280 and GBM166) were predicted to be long-term survivors. Follow up with the patients was continued over time and 1 patient passed away at 6 months, before the 14.6 months of median GBM patient survival threshold. That patient is GBM1295, which MAqCI had correctly identified as a short-term survivor (Table 2). The other 4 patients survived past 14.6 months, making them long-term survivors, which MAqCI had also correctly predicted (Table 2). The prognostic performance of MAqCI for the prospective cohort is illustrated in a heat map similar to the retrospective cohort as described earlier (FIG. 3F), where it is evident that the predicted survival outcome based on the composite MAqCI score matches the actual survival outcome for each patient perfectly (FIG. 4D).

Some may argue that the 14.6 months of median survival threshold for GBM as established by Stupp et al. might be applicable for clinical trial participants which is an selected GBM population, and hence may not be relevant to “real world” patients, whom according to Zhu et al. exhibit an even shorter median survival time of 11 months (Zhu, P., et al., Oncotarget 8, 44015-44031 (2017)). Fourteen and six months was chosen as the cut-off because the analysis was focused on primary GBM patients that had undergone standard of care (surgical resection along with temozolomide (TMZ) and radiation). Unfortunately, the cut-off suggested by Zhu et al., was deemed unsuitable because unlike their study, the study described herein excluded any recurrent patients. Moreover, a small subset of patients in present study received bevacizumab/avastin in addition to TMZ and radiotherapy post-debulking, which was an important criteria for the study by Zhu et al.

TABLE 2

Individual MAqCI measurement metrics, composite MAqCI score and survival outcomes for the prospective GBM patient cohort

| GBM | Highly Motile Cells (%) | Narrow Entry (%) | Highly Motile Ki-67+ (%) | Composite MAqCl Score | Predicted Survival | Actual Survival | Prediction |
|---|---|---|---|---|---|---|---|
| 1295 | 13.6 ± 2.8 | 10.8 ± 4.3 | 71.3 ± 3.3 | 0.96 | Short-Term | Deceased (14.4 Mo) | Correct |
| 1296 | 9.2 ± 1.6 | 15.5 ± 6.8 | 49.4 ± 2.7 | 0.63 | Long-Term | Alive (>14.6 Mo) | Correct |
| 1283 | 12.2 ± 2.4 | 0 | 27.4 ± 6.0 | 0.08 | Long-Term | Deceased (37 Mo) | Correct |
| 1280 | 3.6 ± 1.6 | 13.9 ± 9.0 | 39.6 ± 5.1 | 0.21 | Long-Term | Alive (>14.6 Mo) | Correct |
| 166 | 9.3 ± 1.6 | 18.2 ± 3.5 | 26.9 ± 2.6 | 0.18 | Long-Term | Alive (>14.6 Mo) | Correct |

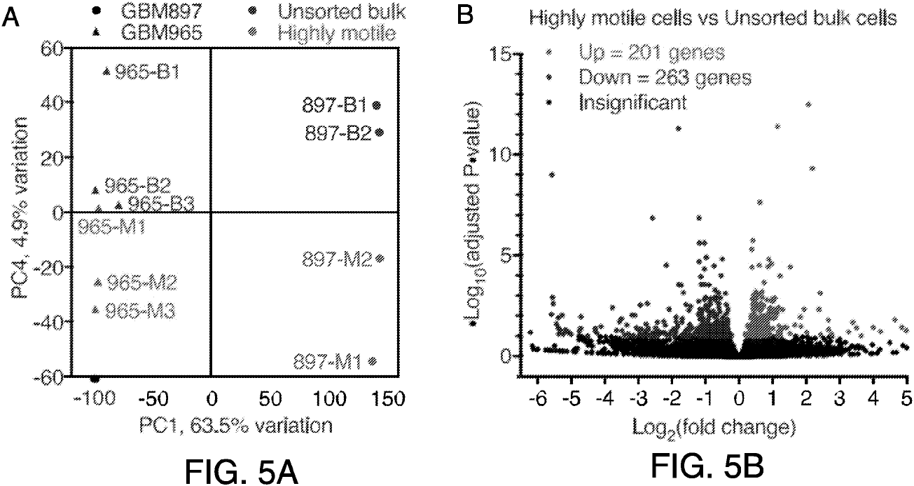

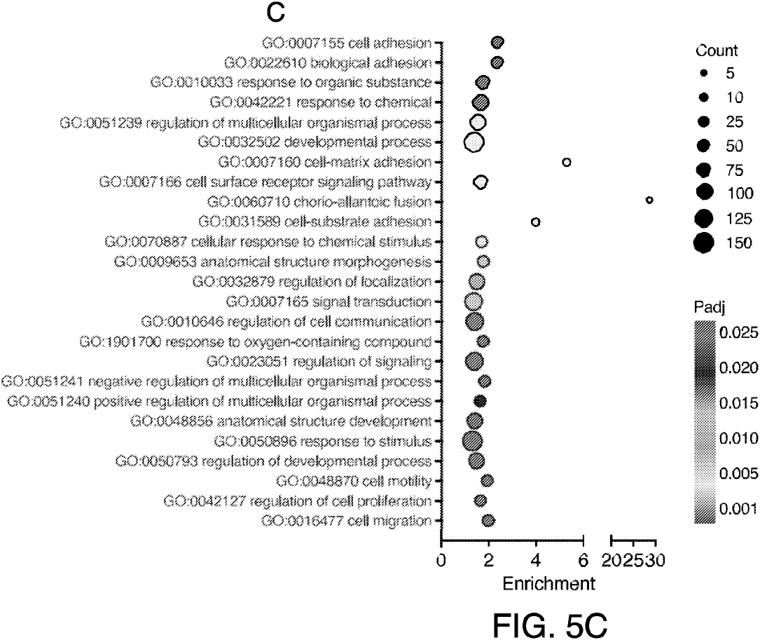

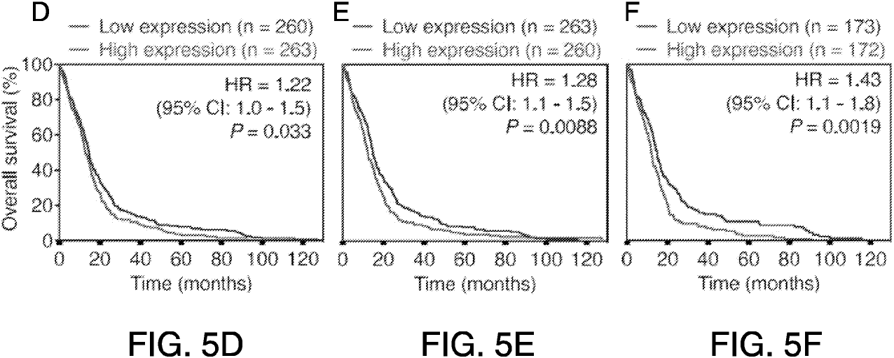

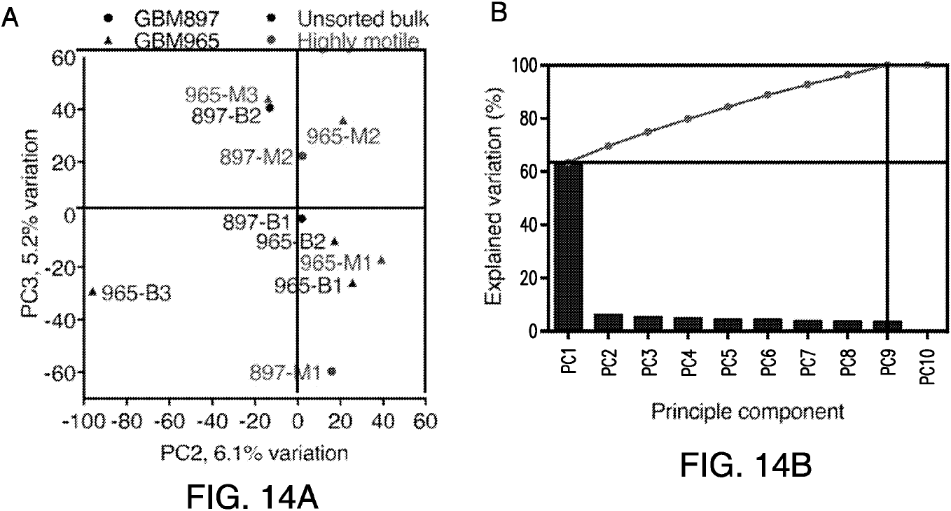

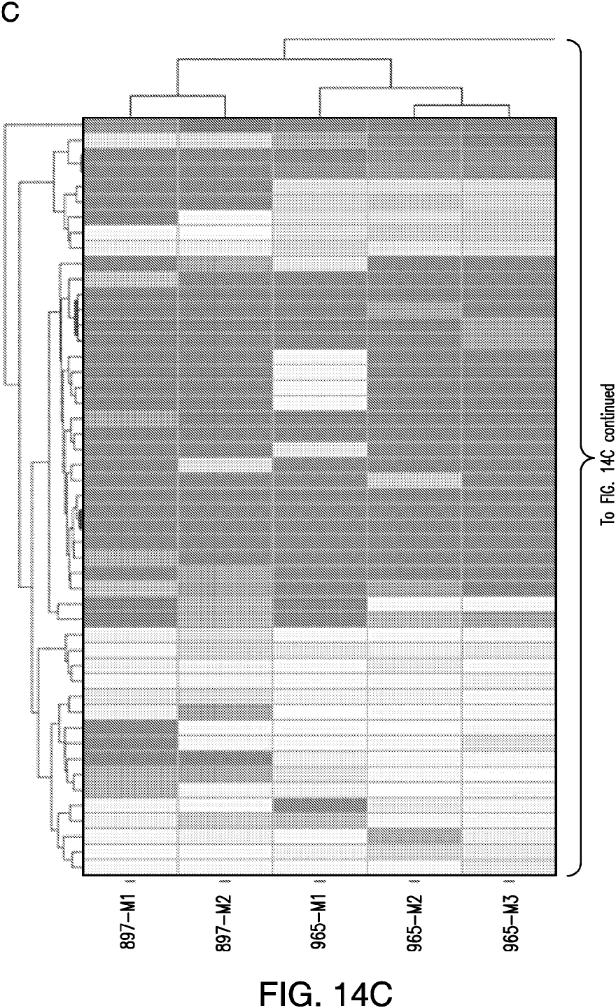

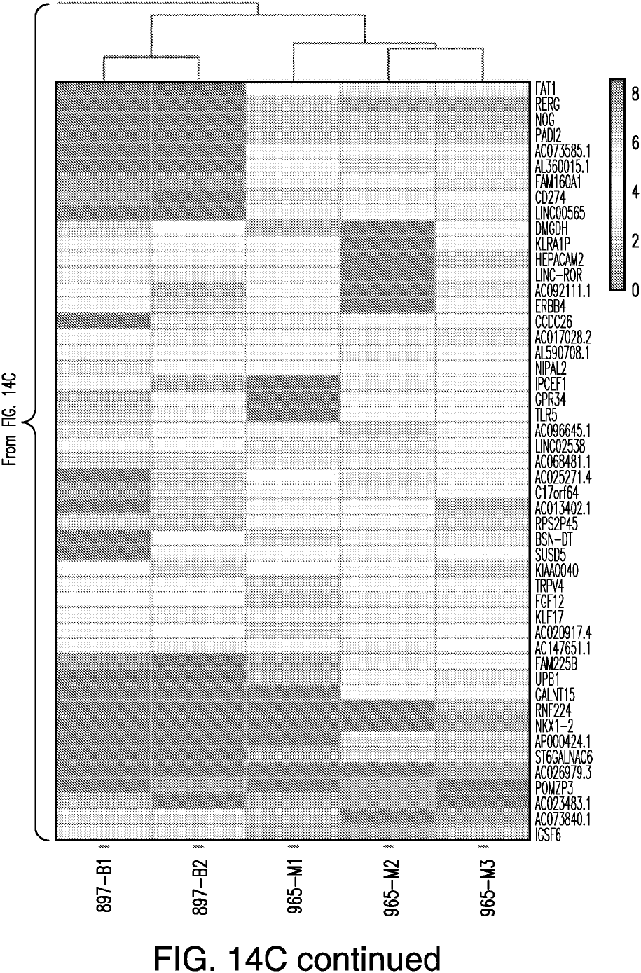

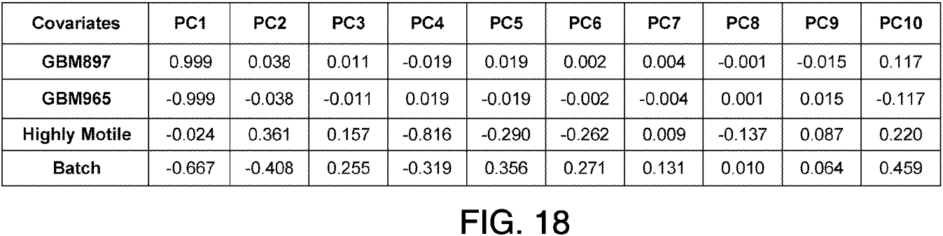

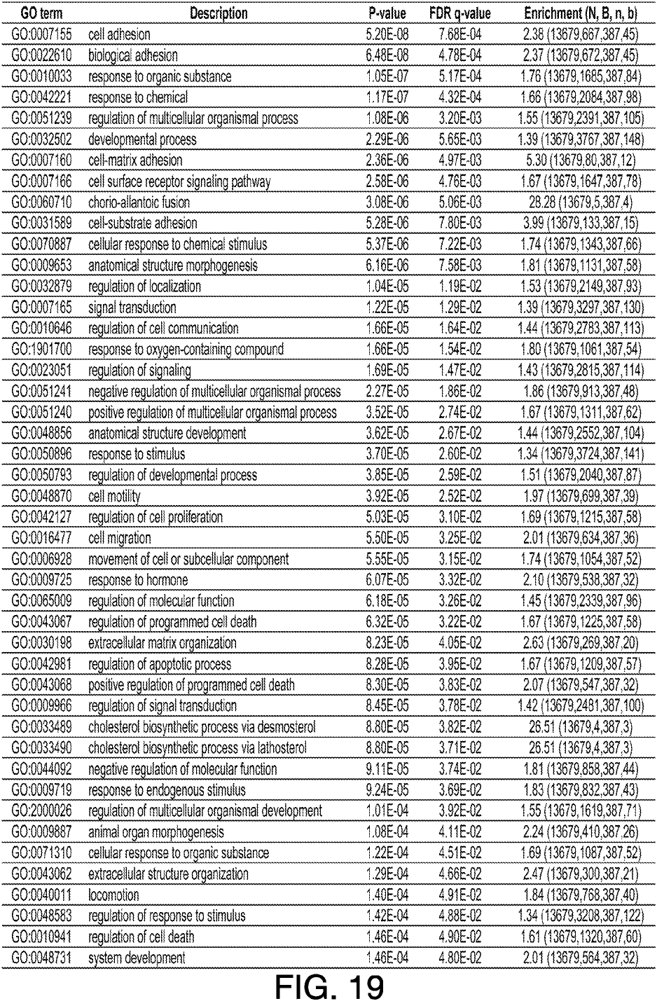

Gene expression profile of the highly motile cell subpopulation identifies DEGs which correlate with poor patient prognosis. In view of the data showing correlation between the abundance of highly motile cells and poor patient prognosis, RNA sequencing (RNAseq) was performed to compare the transcriptomes of the highly motile subpopulation versus unsorted bulk cells from two GBM patients with aggressive disease (GBM965 and 897). Based on a quality threshold of RNA Integrity Number (RIN) of ≥8.4, triplicate sample pairs of highly motile and unsorted bulk cell RNA were collected from GBM965 and duplicate pairs from GBM897. Principal Component Analysis (PCA) identified the inter-tumoral heterogeneity between the samples from the two patients along the first principal component (PC1, R2 = 0.99, padj = 6.9e-13), which accounted for 63.5% of the variation in the dataset (FIG. 5A, FIG. 14B, FIG. 18). The second and third principle components accounted for 6.1% and 5.2% variation, respectively, and did not meaningfully separate the samples by patient or migratory potential (R2 <0.17) (FIGS. 14A-B, FIG. 18). Interestingly, the highly migratory versus unsorted bulk cell specimens from both patients separated along the fourth principle component (PC4, R2 = 0.67, padj = 0.04), which accounted for 4.9% of the variation in the dataset (FIG. 5A, FIG. 18). 464 differentially expressed genes (DEGs, FDR<0.1) were identified in highly motile versus bulk unsorted cells from both patients, with 201 DEGs upregulated and 263 downregulated (FIG. 5B). Unsupervised hierarchical clustering of the 10 specimens based on the top 50 statistically significant DEGs separated the specimens by migratory phenotype and then by patient, and showed clusters of conserved gene expression patterns in the highly motile cells (FIG. 14C). Gene ontology and biological pathway (GOBP) analyses revealed significant enrichment of biological processes related to cell adhesion, signaling, tissue development/morphogenesis, migration, proliferation, and survival in the highly motile cell population (FIG. 5C,, FIG. 19).

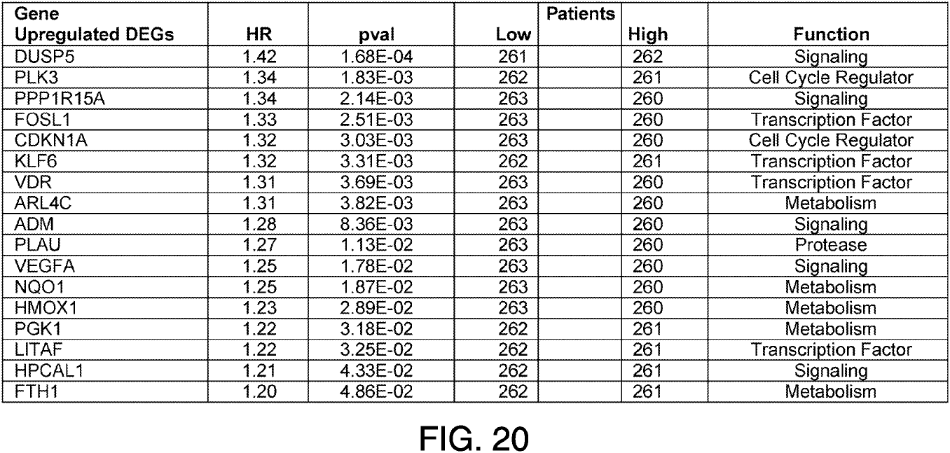

Next, how the gene expression pattern of the highly motile cells was related to the overall survival (OS) of a cohort of 523 GBM patients from The Cancer Genome Atlas program was compared. Of the 464 DEGs identified in the highly motile cells, 261 were found in the microarray data for this cohort. To identify whether patients with high expression of genes upregulated by the migratory cells have a poor prognosis, a composite score was calculated for each patient by summing up the mRNA expression z-score for these DEGs. Patients whose composite scores were above the median value for the cohort were classified as having a high expression of these DEGs. Kaplan Meier survival analysis revealed a trend of reduced OS for upregulated DEGs with padj=0.1, which reached statistical significance (p=0.035) with a hazard ratio (HR) of 1.22 for the 72 upregulated DEGs with a padj<0.05 (FIG. 5D) for which expression data were available. Of note, there was no significance in OS for stratifying patients based on downregulated DEGs. Next, each of the upregulated DEGs identified in the microarray were screened to determine their individual relationship to GBM OS. Stratifying patients based on median gene expression, 20 individual genes significantly (p<0.05) correlated with OS. Importantly, the expression levels of 17 out of the 20 genes (85%) in the highly motile cells relative to the unsorted bulk cell population matched those of GBM patients with lower survival (FIG. 20). Using the collection of these 17 upregulated DEGs, a composite score was calculated for each patient, and were stratified based on median (FIG. 5E) or tercile (highest third versus lowest third) (FIG. 5F) scores. GBM patients with a high composite score in the expression pattern of these 17 upregulated DEGs had significantly worse OS with a HR of 1.28 (FIG. 5E) and 1.43 (FIG. 5F) for the median and tercile scores, respectively, thereby providing further evidence for the highly motile genotype as an indicator of poor prognosis for GBM patients.

Discussion. Cell motility is an important process that contributes to a cancer cell’s ability to disseminate and invade (Paul, C. D. et al., Nat Rev Cancer 17, 131-140, (2017)). In fact, enhanced motility of cancer cells has been widely linked with higher metastatic potential, aggressiveness of cancer and overall poor prognosis (Steeg, P. S., Nat Rev Cancer 16, 201-218 (2016)). As such, the migratory capacity of cancer cells has been proposed and used to correlate with patient and clinical outcomes in a wide variety of cancer types, such as breast and brain cancer cells (Shah, S. R. et al., bioRxiv, 602052, doi:10.1101/602052 (2019), Smith, C. L. et al., Cell Rep 15, 2616-2624 (2016), Liu, J. C. et al., PLoS One 12, e0179223, doi:10.1371/journal.pone.0179223 (2017), and Shah, S. R. et al., Neurosurgery 63, 185-185, (2016)). While previous studies have revealed that migratory behavior of GBM cells is qualitatively instructive in determining tumor aggressiveness (Armento, A. et al., U. in Glioblastoma (ed S. De Vleeschouwer) (2017)), no study or method to date has defined an effective quantitative approach to predicting individual patient prognosis for GBM. Moreover, most of the information regarding cell migration stems from 2D or 3D collagen assays that do not recapitulate the complex in vivo brain tissue microenvironment. In vivo, invasive GBMs have to navigate confining 3D perivascular tracks in brain vessels formed between glial cells and the basement membrane of vascular smooth muscle cells (Gritsenko, P. et al., Histochem Cell Biol 148, 395-406 (2017), and Friedl, P., et al., Cell 147, 992-1009, (2011)). Indeed, prior attempts at examining the relationship between glioma cell migration on 2D surfaces and disease progression have failed to achieve significant predictions to clinically relevant patient features 45. Introducing complexity to the migration assays by examining the migratory behaviors of individual GBM cells on 1D nano-pattern substratum in response to platelet-derived growth factor (PDGF) stimulation allowed successful prediction of GBM location and recurrence potential in a small retrospective cohort of patients (Smith, C. L. et al., Cell Rep 15, 2616-2624 (2016)). However, no significant correlation to patient survival outcomes or prognosis was observed (Smith, C. L. et al., Cell Rep 15, 2616-2624 (2016)). Furthermore, it is unclear whether and how such parameters can independently or in conjunction with other cellular behavior(s) to quantitatively and precisely predict individual patient clinical outcomes for GBM patients.

In MAqCI, the microchannels were designed to recapitulate and mimic important aspects of the complex topography and confining longitudinal pores or perivascular tracks of the brain parenchyma. By examining the migratory behaviors of patient-derived primary GBM cells in response to topographical cues in the absence of any growth factor or chemoattractant stimulation, it was found that the percentage of highly motile cells correlates remarkably well with overall progression-free survival, an unprecedented observation for any in vitro phenotypic-based prognostic assay for GBM. The success of this simple and easy-to-interpret analysis could be attributed to the ability of MAqCI to provide a more physiologically relevant confining microenvironment compared to conventional 1D or 2D migration assays. Along these lines, a significant correlation was also observed between patient survival and the percentage of highly motile cells that entered even more confining 3 µm narrow branches (i.e., percentage of narrow entry), indicating once again the value of subjecting the cells to migrate in a confined microenvironment and how this phenotypic trait can be exploited to determine patient outcome.

Aside from elevated motility, another important hallmark of aggressive cancer is its ability to grow and proliferate uncontrollably. Ki67 is a marker that is commonly used in the clinical setting to assess the proliferation potentials of cancer biopsies via immunohistochemistry staining and has been explored for cancer prognosis application with variable success (Wong, E. et al., Asia Pac J Clin Oncol 15, 5-9 (2019), and Abubakar, M. et al, Breast Cancer Research 18 (2016)). However, owing to the inherent heterogeneity of cancer cells, it is challenging to ascertain if migratory and non-migratory cells display different proliferative potentials and if these potential differences possess any prognostic values. Using MAqCI, the ability to sort the bulk total cancer cell population into different subpopulations based on their motility and assess their Ki67 status independently can be achieved. Notably, if the percentage of Ki67+ cells is quantified for the unsorted bulk cell population, similar to just performing Ki67 staining without the use of MAqCI, any correlation to patient survival cannot be achieved. In stark contrast, the percentage of Ki67+ cells for the highly motile cell subpopulation significantly correlated to GBM patient survival showcasing the importance of MAqCI as a sorting device. Of note, both metrices were unable to achieve significant survival curve separation, unlike the migratory indices, suggesting that proliferation alone is not sufficient for predicting GBM prognosis despite demonstrating some promising correlation to patient survival.

While individually, migration indices, namely percentages of highly motile cells and narrow entry, and proliferative indices, such as percentage of highly motile Ki67+ cells, each possess a certain degree of prognostic values for GBM patient prognosis, they also have their respective shortcomings and, with accuracy values of around 80%, they are not considered clinically useful. Given that GBMs exhibit enhanced cellular proliferation, it is difficult to utilize proliferation index alone as a benchmark for determining individual patient outcomes. Similarly, in order for invasive cells to colonize after they have migrated to distant sites, they need to be able to proliferate in order to establish secondary colonies. To further improve the prognostic performance of MAqCI, the migratory- and proliferative-based indices were combined using logistic regression into a single composite MAqCI score. Remarkably, the composite MAqCI score emerged as the most accurate predictor compared to the individual MAqCI measurement metrics, demonstrating the strongest correlation to patient survival and achieving sensitivity, specificity and accuracy of categorizing patients based on their survival outcomes to close to 90%. Moreover, composite MAqCI score predicts recurrence time successfully in the retrospective patient cohort. Recurrence of GBM following surgical resection represents the primary cause of death in patients and is intimately associated with future patient outcome (Chaichana, K. L. et al, Neuro Oncol 16, 113-122 (2014), and Chaichana, K. L. et al., J Neurosurg 118, 812-820 (2013)). By quantifying the ability of GBMs to both migrate and proliferate, MAqCI is able to capture this aggressive invasive growth behavior and capitalize on this knowledge to make meaningful predictions regarding recurrence time and patient survival. Overall, these results reveal the benefits of combining multiple cellular parameters into making accurate prediction related to patient-specific outcomes and prognosis.

These results also highlight the advantages of MAqCI over traditional non-molecular and molecular characterizations. Notably, demographic, tumor and surgical parameters, such as age, gender, tumor volume, tumor spread, number of surgical resections and KPS score, were non-indicative of GBM patient survival and prognosis. MAqCI also outperforms IDH1 mutation status, which is a clinically utilized independent prognosis indicator for certain subsets of glioma. Interestingly, in the retrospective patient cohort, a complete lack of correlation was observed between patient survival and IDH1 mutation status. Furthermore, a direct comparison between MAqCI and the conventional transwell-migration assay revealed a significant diminished ability for transwell-migration assay to correlate and predict GBM patient survival at comparable time scales. Taken together, the methods and results described herein have demonstrated the superior ability of MAqCI to categorize patients based on their individual survival outcomes over other commonly used non-molecular, molecular as well as migration-based assays.

The retrospective-based findings provide an impetus to further test the efficacy of MAqCI in a prospective manner. As a proof of concept, 5 samples were collected and tested from two different clinical institutions. Out of the 5 prospective patients, 1 patient passed away before the established median threshold of 14.6 months and was hence classified as a short-term survivor, while the other 4 patients were long-term survivors. Remarkably, MAqCI was able to predict the 5 patients specifically and successfully. This suggests the promising potential for MAqCI to be tested in a larger prospective pre-clinical study.

An important feature of MAqCI is its ability to isolate highly motile cells from a heterogeneous tumor cell population for subsequent characterization at the genomic and/or proteomic levels. To illustrate this capability, RNA sequencing of isolated highly motile relative to unsorted bulk cells from two GBM patients was performed, and it was determined that upregulated DEGs with a padj<0.05 correlated with poor GBM OS. A collection of 17 DEGs whose individual expression patterns matched those of GBM patients with reduced overall survival was also identified. Importantly, patients with a high composite score based on the expression of these 17 upregulated DEGs displayed reduced OS. Taken together, these findings provide further support for role of highly motile cells in aggressive GBM progression.

Looking forward, MAqCI has the potential to be used in the clinical setting to rapidly distinguish between aggressive and less-aggressive cancers to inform patient care, management, and potential therapies that can impact the disease. During surgical resection of brain cancer, excess tumor specimen, which is generally discarded as medical waste after allocating a portion for pathological evaluation, can be used to perform MAqCI-related prognostic testing. Such excess specimen is frequently used by research laboratories to establish primary cell lines for future studies. MAqCI technology can thus permit examination of patient-derived cells in order to quantitatively predict patient cancer aggressiveness. Since a significant positive correlation was observed between the predicted survival time (as calculated based on the coefficients of multiple linear regression) and actual patient survival time, future development of this technology may provide non-binary, quantitative read-outs of survival time with increased statistical confidence for each patient. Moreover, MAqCI can serve as a platform for therapeutic screening to determine individual response and identify patient-specific effective therapies that can reduce the abundance of highly motile and proliferative cells. Given the promising performance of MAqCI in GBM, this functional assay may be useful for determining patient surgical and clinical outcomes of other solid cancers, including those with increased propensity to migrate beyond tumor margins and ultimately metastasize to distal sites. MAqCI can also be extended for basic science applications where in depth molecular and genetic characterizations can be performed on highly motile and/or proliferative cells that can be physically isolated from the device following migration.

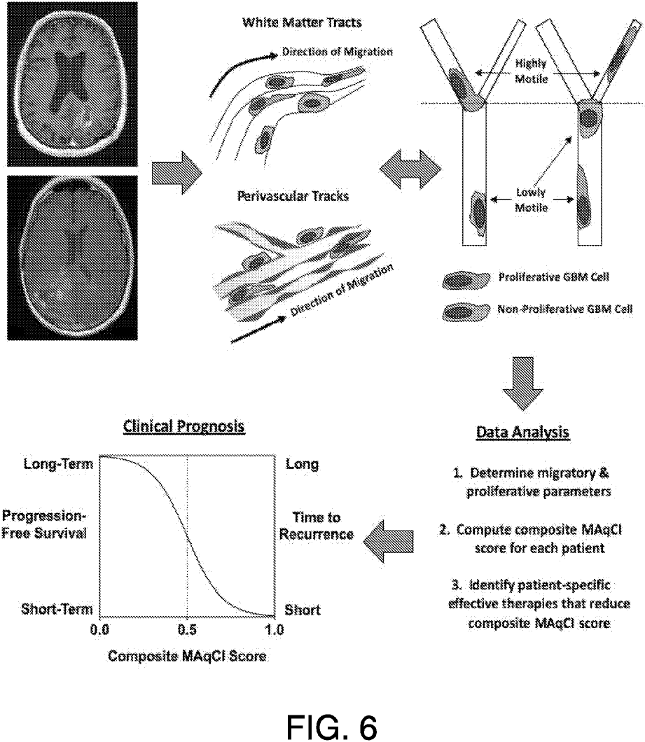

Overall, this study reveals that invasive growth is intimately associated with disease progression and overall patient outcomes. By quantitatively evaluating both migratory and proliferative behaviors of patient-derived primary GBM cells in a physiologically relevant confining microenvironment that mimics the natural invasive routes of native GBM cells, the in vitro testing platform, MAqCI, can precisely determine prognosis in a patient-specific manner (FIG. 6). This in vitro testing platform can provide a useful prognostic tool that can be translated into the clinics to improve personalized management of GBM patients.

Materials and Methods. Cells and cell culture. Patient-derived primary human GBM cells (Retrospective: QNS108, QNS120, GBM153, GBM276, GBM318, GBM496, GBM499, GBM501, GBM549, GBM582, GBM609, GBM612, GBM626, GBM651, GBM653, GBM692, GBM714, GBM724, GBM731, GBM832, GBM847, GBM897, GBM940, GBM960, GBM963, GBM965, GBM1049, GBM1298; Prospective: QNS166, GBM1280, GBM1283, GBM1295, GBM1296) were isolated from primary tumor tissue samples of patients undergoing brain resection surgery for GBM. Tumor samples were pathologically confirmed to be GBM. Tissue donors did not receive any treatment prior to surgery. The primary cells were isolated, purified and maintained through methods that eliminate cross-contamination from other cell types and are capable of maintaining the stemness and molecular characteristics of the original primary tumors (Shah, S. R. et al., Cell Rep 21, 495-507 (2017)). The cells were used for no more than 5 passages after they were thawed from the original frozen stock (passage 1-10). The primary GBM cells were grown as adherent cultures on tissue culture flasks pre-coated with laminin (Trevigen) at a density of 1 µg/cm² surface area diluted with PBS without magnesium and calcium for 3 h at 37° C. The culture media consisted of 1:1 Dulbecco’s Modified Eagle Medium/Nutrient Mixture F-12 (DMEM/F12, Invitrogen), Gem21 Neuroplex™ without vitamin A serum-free supplement (Gemini), 1x antibiotic/antimycotic solution (Sigma-Aldrich), 10 ng/ml of recombinant human fibroblast growth factor (Peprotech) and 20 ng/ml of recombinant human epidermal growth factor (Peprotech). Accutase solution (Sigma-Aldrich) was used to dissociate cells from the laminin-coated tissue culture flasks instead of trypsin.

Microfluidic Assay for Quantification of Cell Invasion (MAqCI). MAqCI comprised a series of 400 µm-long and 10 µm-tall Y-shape microchannels (Yankaskas, C. L. et al, Nat Biomed Eng 3, 452-465 (2019), and Paul, C. D. et al., FASEB J 30, 2161-2170, (2016)), with a 20 µm-wide feeder channel bifurcating at an angle of 65° to 10 µm-wide or 3 µm-wide branches, arrayed perpendicularly between cell seeding (100 µm-wide, 50 µm-high) and media channels (400 µm-wide, 50 µm-high). There are a total of 243 Y-shape microchannels per device spaced at an interval of 50 µm from each other.

MAqCI was fabricated using standard multilayer photolithography and replica molding techniques (Mistriotis, P. et al., J Cell Biol 218, 4093-4111 (2019), Tong, Z. et al., PLoS One 7, e29211, doi:10.1371/journal.pone.0029211 (2012), and Zhao, R. et al., Science Advances 5, eaaw7243 (2019)). The design of the microfluidic device was created in AutoCAD (Autodesk) and transferred to chrome-on-glass darkfield photolithography masks (Photoplot Store). The primary feature of the negative silicon wafer mold, corresponding to the Y-shape microchannels, was fabricated using SU-8 3010 negative photoresist (Microchem). SU-8 3010 was spun to a thickness of 10 µm on a cleaned silicon wafer (University Wafer) with a spin-coater (Single Wafer Spin Processor, Model WS-400A-6NPP-LITE, Laurell Technologies). The film was soft baked and UV- exposed through a photomask defining the Y-shape microchannels array using an EVG620 mask aligner (EVG) at 170 mJ/cm². The exposed wafer was then baked, developed with SU-8 developer, rinsed with isopropanol and dried. To fabricate the cell and medium inlet lines, the photolithography step was repeated using a 50 µm-thick layer of SU-8 3025, with exposure through a mask defining the cells and medium feed lines aligned over primary features at 250 mJ/cm². The completed wafer was passivated by treating with (tridecafluoro-1,1,2,2,- tetrahydrooctyl)-1-trichlorosilane (Pfaltz & Bauer) overnight in a vacuum desiccator.

Completed MAqCI was formed using standard replica molding from the silicon wafer. PDMS elastomer and crosslinker (Sylgard® 184 Silicone Elastomer Kit, Dow Corning) were mixed at a 10:1 w/w ratio, poured over the wafer, degassed in a vacuum, and cured at 85° C. for 2 h. Solidified PDMS were peeled off of the wafer, punched with a 5 mm-diameter hole puncher at the designated well inlets and outlets and cut into appropriate sizes. The cut PDMS devices and 25 mm x 75 mm microscope slides (Electron Microscopy Sciences) were cleaned with 100% ethanol, blown dry with filtered air, and treated with oxygen plasma (Plasma Cleaner PDC-32G, Harrick Plasma) for 2 min at 18W to render the surfaces hydrophilic. The plasma treated PDMS devices were subsequently attached and sealed to the glass slides. To enable cell binding and adhesion, each MAqCI device was coated with 12 µg/ml of laminin 1 (Trevigen) diluted in PBS without magnesium and calcium at 37° C. for 1 h followed by 4° C. overnight.

MAqCI assay. Patient-derived primary GBM was detached from laminin-coated tissue culture flask with Accutase, counted and resuspended to a final concentration of 1 × 106 cells/ml. Prior to cell seeding, laminin coating solution was aspirated from the microchannels and the devices were washed once with PBS without magnesium and calcium. Thirty microliters were added to the bottom most medium inlet reservoir as backpressure to prevent the cells from prematurely traversing the Y-shape microchannels by convective flow. Fifty microliters of cell suspension, which is equivalent to 5 × 105 cells, was then introduced to the right cell seeding inlets and the cells were allowed to incubate at 37° C. for 5 min to allow for attachment and seeding at the entrance of the Y-shape microchannels. After 5 min, 30 µl was transferred to the left cell seeding inlets to enable cell flow and seeding from the other direction, followed by another 5 min of incubation. Afterwards, cell suspension was removed and transferred from the right cell seeding inlets and the left cell seeding inlets, and the cells were then incubated again for another 5 min. The remaining cells in the cell inlets reservoir were then removed. One hundred microliters of GBM media were then introduced to each of the three medium inlet reservoirs and also the cell seeding inlets on the right side of the device. Migration of GBM in MAqCI was visualized and recorded via time-lapse live microscopy via software-controlled stage automation. The cells were imaged via a 10x/0.30 numerical aperture Ph1 objective lens every 20 min for 24 h using a Digital Sight Qi1Mc camera mounted on a Nikon Inverted microscope equipped with a stage top incubator (Tokai Hit Co., Shizuoka, Japan) maintained at 37° C. with 5% CO2 and humidity.

The video was inspected visually and analyzed using the NIS Elements Viewer to quantify the number of highly motile and lowly motile cells. Highly motile cells are defined as cells that migrate up the feeder channels, reach the bifurcation and enter either one of the two branches. Conversely, lowly motile cells include the cell population that enter and migrate in the feeder channels but do not enter the bifurcations. Tracking for a cell ceases either after more than half of the cell body has entered the bifurcations or has exited the bottom of the feeder channels. Cells are excluded from analysis if they 1) started already more than halfway in the feeder channels at the beginning of the experiment; 2) undergo cell division; 3) exited and reenter the microchannels. The percentage of highly motile cells was calculated as the ratio of highly motile cells over the sum of both highly motile and lowly motile cells. The number of cells that either enter the 3 µm or and 10 µm-wide branches were also recorded for the calculation of percentage of narrow entry, which is defined as the percentage of highly motile cells that enter the 3 µm narrow channels.

Ki67 Immunofluorescence staining. Patient-derived primary GBM cells were seeded into MAqCI as per the protocol used for the migration study and allowed to migrate in MAqCI for 24 h at 37° C. Cells were fixed in 4% paraformaldehyde for 20 min, permeabilized in 1% Triton X-100 for 10 min and blocked for 2 h in blocking buffer comprising PBS without magnesium and calcium with 2% bovine serum albumin and 0.1% Triton X-100. Cells were incubated with Ki67 (8D5) mouse monoclonal antibody (Cell Signaling, 9449S, 1:800) diluted in blocking buffer at 4° C. overnight, followed by 2 h incubation with Alexa Fluor 488 goat anti-mouse secondary antibody (Invitrogen, A11001, 1:200) and Hoechst 33342 (ThermoFisher Scientific, H3570, 1:2000) diluted in blocking buffer in room temperature protected from light. The cells were washed thoroughly with PBS without magnesium and calcium between each step. Imaging of the immunostained samples was performed on an inverted Eclipse Ti epifluorescence microscope (Nikon) with a 10x/0.30 numerical aperture lens. The percentage of Ki67-positive cells was calculated for the highly motile cells (i.e., percentage of highly motile Ki67-positive cells) and for the cells that enter the Y-shape microchannels (i.e., percentage of unsorted Ki67-positive cells).

Western blot and antibodies. Standard western blot techniques were performed ⁵². The antibodies used are listed below. Primary antibodies: 1) IDH1 (D2H1) rabbit mouse monoclonal antibody (Cell Signaling, 8137S, 1:1000); 2) anti-IDH1 R132H mouse monoclonal antibody (Sigma Aldrich, SAB4200548, 1:250); and 3) GAPDH (14C10) rabbit monoclonal antibody (Cell Signaling, 2118S, 1:2000). Secondary antibodies: 1) anti-mouse IgG, HRP-linked antibody (Cell Signaling, 7076S, 1:2000); and 2) anti-rabbit IgG, HRP-linked antibody (Cell Signaling, 7074S, 1:2000).

Transwell-migration assay. Transwell-migration of the retrospective patient-derived primary cells were monitored using the xCELLigence RTCA DP instrument (Acea Biosciences, Inc.) according to the manufacturer’s protocol using a CIM-plate 16 chambers30. These plates have chambers that are similar to Boyden chambers; they consist of an upper chamber where the GBM cells are seeded in serum-free DMEM/F12, a microporous polyethylene terephthalate (PET) membrane with an average pore diameter of 8 µm (through which GBM cells migrate to the lower chamber), electrodes directly below this membrane, and a lower chamber which is filled with DMEM/F12 containing Gem21 Neuroplex supplemented with EGF and FGF as chemoattractant. 4 × 10⁴ cells per well were seeded into the upper chamber of a CIM-plate 16 chambers. The extent of GBM cell migration was monitored in real time as cell index for 48 h at 15 min intervals in a humidified incubator maintained at 37° C. and 5% CO₂ by measuring changes in electrical impedance with electrodes that are attached directly underneath the PET membrane. Each patient-derived primary GBM specimen was run in triplicate for each experiment.

Correlation between in vitro MAqCI and clinical data. To assess the relationship between the migratory and proliferative measurements obtained with MAqCI to clinical patient outcome, the samples were separated into short- and long-term survival groups based on the threshold of 14.6 month of median GBM patient survival established by Stupp et al. (Stupp, R. et al., N Engl J Med 352, 987-996 (2005)). Student’s t-test was conducted to detect statistical difference between the MAqCI measurement metrics between the short- versus long-term survival groups. Additionally, Pearson’s correlation analysis was conducted to assess the linear correlation between the MAqCI measurement metrics and patient survivals (in months).

Identification of optimal threshold. The thresholds of the in vitro MAqCI measurement metrics were systematically varied. For each threshold value, the percentage of the in vitro MAqCI measurement metric was compared against to classify the samples into either long-term survivors (< threshold) or short-term survivors (≥threshold). MAqCI’s prediction was then compared to the actual patient survival months which have been previously stratified into short- and long-term survivors based on the 14.6 months median GBM patient survival established by Stupp et al. (Stupp, R. et al., N Engl J Med 352, 987-996 (2005)), to label each prediction as true positive, true negative, false positive, or false negative (where true represents a match between MAqCI’s prediction and actual patient survival, and positive/negative denotes short- or long-term survivors, respectively). With these classifications, the prediction performance characteristics, including sensitivity, specific, PPV, NPV and accuracy, of the MAqCI measurement metric at that particular threshold value was computed. This process is iterated for the entire range of each MAqCI measurement metric and the optimal threshold value was selected at a value that maximizes the average of the prediction performance characteristics.

Assessment of the prognostic value of MAqCI measurement metrics. The patient survival data was plotted as a Kaplan-Meier graph and the mean survival time in months of patients were compared between the groups separated by the optimal threshold determined. Log-Rank (Mantel Cox) test was conducted to detect statistical significance between the two survival curves. The performance characteristics, including sensitivity, specificity, PPV, NPV, accuracy and area under curves of receiver operating characteristic curve, of each of the MAqCI measurement metric in successfully classifying patients into either short- or long-term survivors, was computed and tabulated. Additionally, the time to recurrence of the samples was compared between groups as separated by the optimal threshold. Similar Kaplan-Meier curve comparisons were also made with demographic, surgical and tumor characteristic information collected from the patients to assess the prognostic values of these other clinically available indices.

Logistic Regression. A composite MAqCI score that combines the individual MAqCI measurement metrics was computed via logistic regression, where the probability of each sample belonging to the short-term survival group (i.e., <14.6 months) was calculated based on the predictors (Xi): percentage of highly motile cells, percentage of narrow entry and percentage of highly motile Ki67-positive cells (equation 1). Logistic regression coefficients (bi) were determined in MATLAB using the glmfit function for the retrospective cell lines (n=28) based on the short- and long-term GBM survival stratification of 14.6 months (FIG. 16). Probability values, namely composite MAqCI scores, were calculated in MATLAB using the glmval function. Similar threshold identification, correlation and Kaplan-Meier curve comparison analyses as described above were repeated with the composite MAqCI score to evaluate its prognostic performance.

$$s c o r e = \frac{{e}^{\left({b}_{0} + {b}_{1} {X}_{1} + {b}_{2} {X}_{2} + {b}_{3} {X}_{3}\right)}}{1 + {e}^{\left({b}_{0} + {b}_{1} {X}_{1} + {b}_{2} {X}_{2} + {b}_{3} {X}_{3}\right)}} \qquad \text{(Equation 1)}$$

Where b0 = -6.893, b1 = 0.137, X1 = % migratory cells, b2 = 0.072, X2 = % narrow entry, b3 = 0.103, X3 = % highly motile KI67+ cells. Note: X1-3 are the device measurements for each patient, while b0-3 are constants.

Isolation of highly motile cells. Approximately 5 × 10⁵ patient-derived primary GBM cells (specifically GBM897 and GBM965) were seeded in MAqCI and allowed to travel through the microchannels for 48 h. Highly motile cells that have successfully exited the branch channels and entered the collection channel were first washed with PBS and then dissociated from the device with TrypLE Express reagent (Gibco) for 10 min. A microbore tubing connected to a 10 ml syringe filled with regular GBM culture media was attached to a collection inlet. Pressure was then applied to push the media from the syringe through the collection channel to force the detached highly motile cells into the collection outlet. These cells were subsequently collected with a micropipette and transferred to a 15 ml tube with 2 ml of regular GBM culture media. Typically, each MAqCI device produced around 50-100 highly motile cells. Highly motile cells from multiple MAqCI were pooled to yield a greater number of cells for downstream processing for RNAseq. Similar procedures were repeated for cells seeded in the cell seeding channels to obtain an unsorted bulk population as control. The number of unsorted cells were normalized and diluted accordingly to the number of isolated highly motile cells, as enumerated manually during the isolation process by microscope observation.

RNAseq and analysis. Total RNA was purified from equal numbers of highly motile or unsorted bulk cells using the RNeasy Micro Kit (Qiagen). RNA was evaluated by Bioanalyzer using the Agilent RNA 60000 Pico kit. Samples with an RNA Integrity Number (RIN) of 8.4-9.3 were used. Three hundred and thirty-five pg of RNA of each sample were used to prepare complementary DNA (cDNA) libraries using the Smart-seq v4 kit (Takara). cDNAs were purified by AMPure XP beads, fragmented by sonication to ~400 bp, and subjected to barcoding and single-end sequenced on an Illumina NextSeq 500 with 75 cycles. RNA-seq reads were mapped to hg38 reference genome using HISAT253 aligner. HTSeq framework (Anders, S., Pyl, P. T. & Huber, W. Bioinformatics 31, 166-169 (2015)) was used to quantify read counts per gene from aligned reads using human GENCODE release 33 (GRCh38.p13) gene models. The Bioconductor/R packages DESeq2_1.26.055 was used for normalization and differential gene expression analysis. Principal component analysis was performed on regularized logarithm (rlog) transformed counts. GO term enrichment analysis was performed using GOrilla database (Eden, E., et al., BMC Bioinformatics 10, 48, (2009), and Eden, E., et al., PLoS Comput Biol 3, e39, doi:10.1371/journal.pcbi.0030039 (2007)).

Survival analysis based on patient gene expression pattern. Patient survival and gene expression data for 12,042 genes based on microarray analysis from the TCGA Firehose Legacy cohort were downloaded from cBioPortal. Using a custom MATLAB code, each patient’s z-score for each upregulated DEG of interest was summed up to create a composite score for that patient. Patients with composite scores above the median composite score for the cohort were classified as having high expression. For select analyses, the upper and lower tercile of composite scores were used to stratify the patients instead of the median. The code then tabulated the survival data for patients in both groups. Then, GraphPad Prism was used to generate Kaplan-Meier survival plots, log-rank statistical tests, hazard ratios and 95% confidence intervals of the hazard ratios.

Statistical analysis. Data are presented as mean±S.E.M. from n≥3 independent experiments unless otherwise stated. Graphing and statistical analyses were performed with GraphPad Prism 7 (Graphpad Software). Statistical significance was determined between pairs of data with an unpaired student’s t-test for continuous variables and Fisher’s exact test for categorical variables. Pearson’s correlation was used to assess the degree of correlation between two continuous variables. Two-tailed log-rank (Mantel-Cox) test was employed to assess the statistical difference between two Kaplan-Meier survivor curves. For principal component correlation analysis, a two- tailed t-test was performed for Pearson’s correlation coefficient R, which follows a t-distribution with n-2 degrees of freedom as described by equation 2.

$$t = \frac{R \sqrt{n - 2}}{\left(\sqrt{1 - {R}^{2}}\right)} \qquad \text{(Equation 2)}$$

Data availability. RNA sequencing data are available at the National Center for Biotechnology Information Gene Expression Omnibus under accession number GSE144610.

### Example 2: Identification of 9 Overlapping DEGs in Breast Cancer

Methods. RNA sequencing (RNAseq) was performed to compare the transcriptomes of the highly motile subpopulation versus unsorted bulk cells from two GBM patients with aggressive disease (GBM965 and 897). Bioinformatics analytics (Principal Component Analysis (PCA)) identified 464 differentially expressed genes (DEGs, FDR<0.1) in highly motile versus bulk unsorted cells from both patients, with 201 DEGs upregulated and 263 downregulated. The gene expression pattern of the highly motile cells was then related to the overall survival (OS) of a cohort of 523 GBM patients from The Cancer Genome Atlas program. Of the 464 DEGs identified in the highly motile cells, 261 were found in the microarray data for this cohort.

To identify whether patients with high expression of genes upregulated by the migratory cells have a poor prognosis, a composite score was calculated for each patient by summing up the mRNA expression z-score for these DEGs. Patients whose composite scores were above the median value for the cohort were classified as having a high expression of these DEGs.

Kaplan Meier survival analysis revealed a trend of reduced OS for upregulated DEGs with p_{adj}=0.1, which reached statistical significance (p=0.033) with a hazard ratio (HR) of 1.22 for the 72 upregulated DEGs with a p_{adj}<0.05 (FIG. 5D) for which expression data were available.

Next each of the upregulated DEGs identified in the microarray were screened to determine their individual relationship to GBM OS. Stratifying patients based on median gene expression, 20 individual genes significantly (p<0.05) correlated with OS. Importantly, the expression levels of 17 out of the 20 genes (85%) in the highly motile cells relative to the unsorted bulk cell population matched those of GBM patients with short-term survival.

Using the collection of these 17 upregulated DEGs, a composite score was calculated for each patient, and stratified them based on median (FIG. 5E) or tercile (highest third versus lowest third) (FIG. 5F) scores. GBM patients with a high composite score in the expression pattern of these 17 upregulated DEGs had significantly worse OS with a HR of 1.28 and 1.43 for the median and tercile scores, respectively, thereby providing further evidence for the highly motile genotype as an indicator of poor prognosis for GBM patients.

Next, each of the 17 upregulated DEGs identified in the highly motile subpopulation of GBM patients were screened to determine their individual relationship to breast cancer overall survival (OS). From these 17 DEGs, nine overlapping DEGs were found whose upregulation correlates with poor prognosis (low OS) in breast cancer. The list and function of these 9 genes along with relevant pharmacological inhibitors are shown in Table 2. Specifically, three genes (PGK1, NQO1 and HMOX1) are involved in metabolism; three genes (VEGFA, ADM and HPCAL1) are involved in signaling; PLK3 is a cell cycle regulator; FOSL1 is a transcription factor; and PLAU is a protease.

TABLE 2

Overlapping DEGs for breast and GBM cancers with primary function

| | |
|---|---|
| GENE | FUNCTION |
| PGK1 | Metabolism |
| NQO1 | Metabolism |
| HMOX1 | Metabolism |
| VEGFA | Signaling |
| ADM | Signaling |
| HPCAL1 | Signaling |
| PLK3 | Cell cycle regulator |
| FOSL1 | Transcription factor |
| PLAU | Protease |

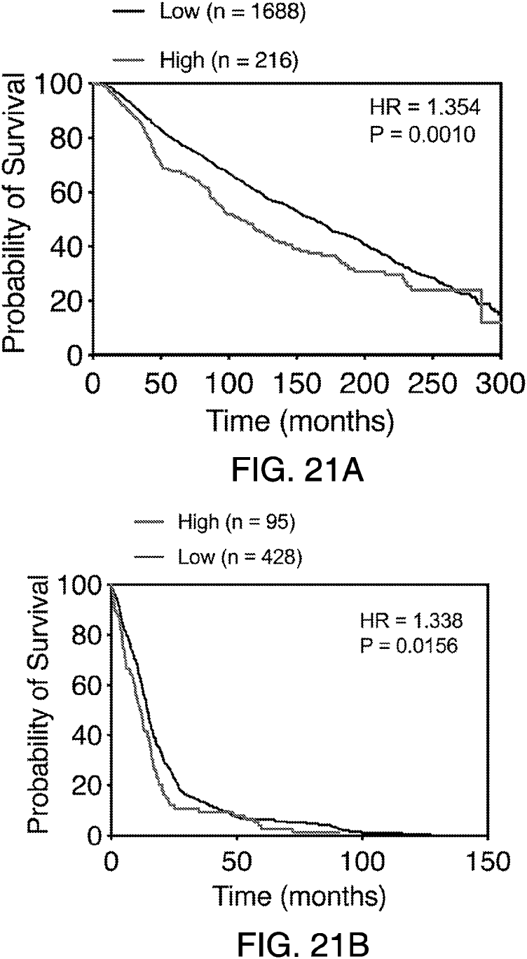

FIG. 21 shows that using the methods described herein for the collection of the three genes involved in metabolism (PGK1, NQO1 and HMOX1), it was found that high expression of these 3 metabolic DEGs correlates with reduced OS in both breast cancer (HR=1.354) and GBM (HR=1.338) datasets.

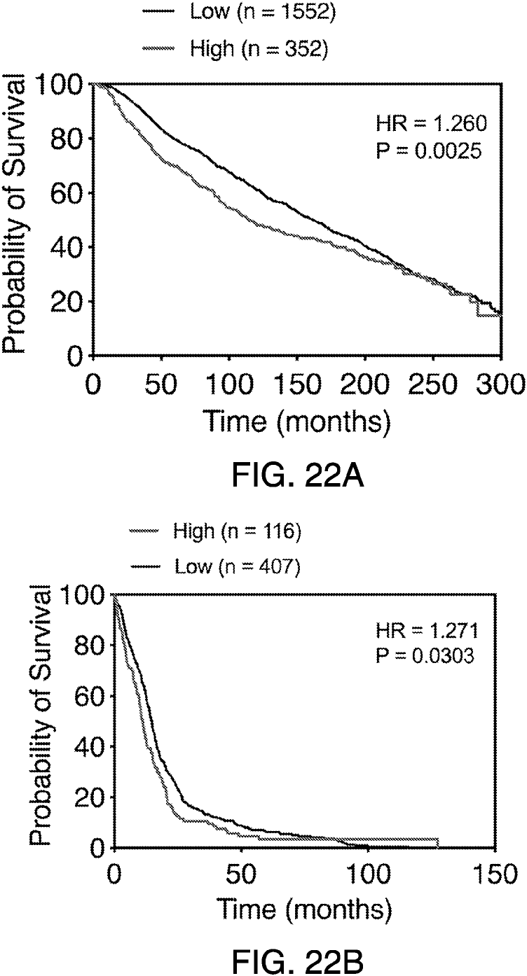

FIG. 22 shows that using the methods described herein for the collection of the three genes involved in signaling (VEGFA, ADM and HPCAL1), it was found that high expression of these 3 signaling DEGs correlates with reduced OS in both breast cancer (HR=1.26) and GBM (HR=1.271) datasets.

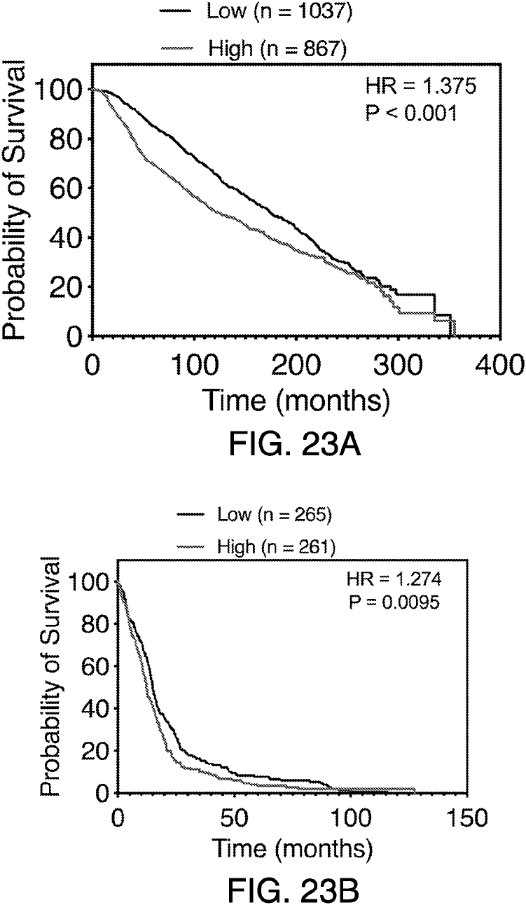

FIG. 23 shows that using the methods described herein for the collection of the 9 overlapping genes, it was found that high expression of these 9 DEGs correlates with reduced OS in both breast cancer (HR=1.375) and GBM (HR=1.274) datasets.

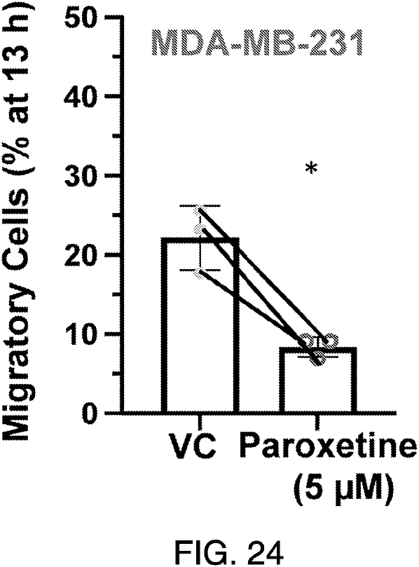

Table 3 shows a list of pharmacological inhibitors that may be useful in increasing the probably of survival in breast cancer or GBM patients that show a higher expression of one or more of the following genes: PGK1, NQO1, HMOX1, VEGFA, ADM, HPCAL1, PLK3, FOSL1, and PLAU. FIG. 24 shows that paroxetine (5 µM) reduces the percentage of migratory cells in MAqCI at 13 hours post administration compared to vincristine (VC) in a breast the metastatic cancer cell line MDA-MB-231 down to the levels of non-metastatic cells. These data demonstrate that targeting one or more of PGK1, NQO1, HMOX1, VEGFA, ADM, HPCAL1, PLK3, FOSL1, and PLA U can reduce the metastasis.

TABLE 3

List of pharmacological inhibitors useful in increasing probably of survival for breast and GBM cancers

| | | |
|---|---|---|
| GENE | FUNCTION | PHARMACOLOGICAL INHIBITOR |
| PGK1 | Metabolism | |
| NQO1 | Metabolism | Dicumarol |
| HMOX1 | Metabolism | Tin protoporphyrin IX dichloride |
| VEGFA | Signaling | Bevacizumab, ranibizumab |
| ADM | Signaling | Paroxetine HCl |
| HPCAL1 | Signaling | |
| PLK3 | Cell cycle regulator | Rigosertib, fostamatinib |
| FOSL1 | Transcription factor | |
| PLAU | Protease | Amiloride, urokinase |

What is claimed is:

1-27. (canceled)

28. A method of treating a subject with an increased risk of recurrence of glioblastoma, the method comprising:
a) obtaining a brain tissue sample or having obtained a brain tissue sample from a subject;
b) determining gene expression levels of DUSP5, PLK3, PPP1R15A, FOSL1, CDKNIA, KLF6, VDR, ARL4C, ADM, PLAU, VEGFA, NQOI, HMOX1, PGK1, LITAF, HPCAL1 and FTH1 in the sample from the subject;
c) identifying the subject to have an increased risk of recurrence of glioblastoma when the level of gene expression of DUSP5, PLK3, PPP1R15A, FOSL1, CDKN1A, KLF6, VDR, ARL4C, ADM, PLAU, VEGFA, NQOI, HMOX1, PGK1, LITAF, HPCAL1 and FTH1 in the sample is determined to be higher than a predetermined reference level of gene expression of DUSP5, PLK3, PPP1R15A, FOSL1, CDKN1A, KLF6, VDR, ARL4C, ADM, PLAU, VEGFA, NQOI, HMOX1, PGK1, LITAF, HPCAL1 and FTH1; and
d) administering a suitable cancer therapeutic to the subject having an increased risk of recurrence of glioblastoma.

29. (canceled)

30. A method of treating a subject with an increased risk of recurrence of breast cancer, the method comprising:
a) obtaining a breast tissue sample or having obtained a breast tissue sample from a subject;
b) determining gene expression levels of PLK3, FOSL1, ADM, PLAU, VEGFA, NQO1, HMOX1, PGK1, and HPCAL1 in the sample from the subject;
c) identifying the subject to have an increased risk of recurrence of glioblastoma when the level of gene expression of PLK3, FOSL1, ADM, PLAU, VEGFA, NQO1, HMOX1, PGK1, and HPCAL1 in the sample is determined to be higher than a predetermined reference level of gene expression of PLK3, FOSL1, ADM, PLAU, VEGFA, NQOI, HMOX1, PGK1, and HPCAL1; and
d) administering a suitable cancer therapeutic to the subject having an increased risk of recurrence of breast.

31. (canceled)

32. (canceled)

33. The method of claim 28, wherein the sample is from a subject undergoing brain resection surgery.

34. The method of claim 28, wherein the subject has been diagnosed with glioblastoma.

35. The method of claim 30, wherein the sample is from a subject undergoing breast conserving surgery.

36. (canceled)

37. The method of claim 30, wherein the subject has been diagnosed with breast cancer.

38. (canceled)

39. The method of claim 28, further comprising determining the invasiveness of a call or a population of cells from the brain tissue sample.

40. The method of, wherein steps a) and b) are repeated.

41. (canceled)

42. (canceled)

43. The method of claim 28, wherein the selected cancer therapeutic comprises chemotherapy, radiation therapy, or therapy targeted to specific pathways known to be important in glioblastoma or the immune system.

44. (canceled)

45. (canceled)

46. The method of claim 30, wherein the selected cancer therapeutic comprises chemotherapy, radiation therapy, or therapy targeted to specific pathways known to be important in breast cancer or the immune system.

47-52. (canceled)

53. A method of screening for a therapeutic agent, the method comprising:
a) placing a cell or a population of cells from a brain or breast tissue sample in an integrative microfluidic apparatus, wherein the integrative microfluidic apparatus comprises a migratory channel and a bifurcation point in the channel;
b) determining whether the cell or the population of cells migrates through the migratory channel of the apparatus and to the bifurcation point of the channel in the presence and absence of the therapeutic agent; and
c) determining that the therapeutic agent is an inhibitor of cancer cell migration when the cell or population of cells does not migrate through the migratory channel of the apparatus and to the bifurcation point of the channel in the presence of the therapeutic agent.

54. The method of claim 53, wherein brain tissue sample is from a subject diagnosed with glioblastoma.

55. The method of claim 53, wherein breast tissue sample is from a subject diagnosed with breast cancer.

56. The method of claim 53, wherein the brain tissue sample comprises a higher level of gene expression of DUSP5, PLK3, PPP1R15A, FOSL1, CDKNIA, KLF6, VDR, ARL4C, ADM, PLAU, VEGFA, NQOI, HMOX1, PGK1, LITAF, HPCAL1 and FTH1 compared to a predetermined reference level of gene expression of DUSP5, PLK3, PPP1R15A, FOSL1, CDKNIA, KLF6, VDR, ARL4C, ADM, PLAU, VEGFA, NQOI, HMOX1, PGK1, LITAF, HPCAL1 and FTH1.

57. The method of claim 53, wherein the brain tissue sample comprises a higher level of gene expression of PLK3, FOSL1, ADM, PLAU, VEGFA, NQOI, HMOX1, PGK1, and HPCAL1 compared to a predetermined reference level of gene expression of PLK3, FOSL1, ADM, PLAU, VEGFA, NQOI, HMOX1, PGK1, and HPCAL1.

58. The method of claim 30, wherein steps a) and b) are repeated.

59. The method of claim 30, further comprising determining the invasiveness of a cell or a population of cells from the breast tissue sample.

60. The method of claim 59, wherein the cell or the population of cells from the brain tissue sample are incubated and imaged in an integrative microfluidic apparatus, and determining whether the cells or population of cells in the sample are invasive when the cell or population of cells migrates through the migratory channel of the apparatus and to the bifurcation point of the channel.

61. The method of claim 28, wherein the cell or the population of cells from the brain tissue sample are incubated and imaged in an integrative microfluidic apparatus, and determining whether the cells or population of cells in the sample are invasive when the cell or population of cells migrates through the migratory channel of the apparatus and to the bifurcation point of the channel.
